(19) United States
(12) Patent Application Publication
(10) Pub. No.: US 2009/0074766 A1
(43) Pub. Date: Mar. 19, 2009

(54) Methods of inhibiting HIV-2 infection

(76) Inventors: Thomas J. Ketas, Dumont, NJ (US); William C. Olson, Yorktown Heights, NY (US)

Correspondence Address: COOPER & DUNHAM, LLP, 30 Rockefeller Plaza, 20th Floor, NEW YORK, NY, 10112, US

(21) Appl. No.: 12/283,482

(22) Filed: Sep. 12, 2008

Related U.S. Application Data
(60) Provisional application No. 60/993,804, filed on Sep. 14, 2007.

(51) Int. Cl. A61K 39/395

(52) U.S. Cl. 424/133.1; 424/144.1

(57) ABSTRACT

This invention provides methods of inhibiting HIV-2 infection of a susceptible cell by HIV-2 which comprises subjecting the susceptible cell to an effective HIV-2 infection inhibiting dose of a humanized antibody designated PRO 140, or of an anti-CCR5 receptor monoclonal antibody, wherein the effective HIV-1 infection inhibiting dose comprises from 0.1 mg per kg to 25 mg per kg of the subject's body weight, so as to thereby inhibit the infection of the susceptible cell by HIV-2.

This invention was made with support under United States Government Grant No. AI066329 from the National Institute of Allergy and Infectious Diseases. Accordingly, the United States Government has certain rights in the subject invention.

Throughout this application, various publications are referenced in parentheses by author name and date, or by a patent or patent publication number. Full citations for these publications may be found at the end of the specification immediately preceding the claims. The disclosures of each of these publications in its entirety are hereby incorporated by reference into this application in order to more fully describe the state of the art as known to those skilled therein as of the date of this application.

### BACKGROUND OF THE INVENTION

In 1984, 3 years after the first reports of a disease that was to become known as AIDS, researchers discovered the primary causative viral agent, the human immunodeficiency virus type 1 (HIV-1). Human immunodeficiency virus type 2 (HIV-2) was identified in West Africa in 1986 (Clavel et al. 1986 Isolation of a new human retrovirus from West African patients with AIDS Science 233:343-346) The similarity of HIV-2 to HIV-1 has been investigated. HIV-1 and HIV-2 share genetic and biological properties, such as genome structure, mechanisms for transactivation, and CD4 cell depletion (Guyader et al. 1987 Genome organization and transactivation of the human immunodeficiency virus type 2 Nature 326:662-669) HIV-1 and HIV-2 have the same mode of transmission and are associated with similar opportunistic infections and AIDS. In persons infected with HIV-2, immunodeficiency seems to develop more slowly and to be milder. Compared with persons infected with HIV-1, those with HIV-2 are less infectious early in the course of infection due to a longer clinical latency period (Ancelle et al. 1987 Long incubation period of HIV-2 infection Lancet i:688-689). As the disease advances, HIV-2 infectiousness seems to increase; however, compared with HIV-1, the duration of this increased infectiousness is shorter and the viral load in the asymptomatic stage is lower (Simon et al. 1993 Cellular and plasma viral load in patients infected with HIV-2 AIDS 7:1411-1417). HIV-1 and HIV-2 also differ in geographic patterns of infection, which are predominantly found in Africa.

Infection of cells by HIV-1 or HIV-2 is mediated by the viral envelope (Env) glycoproteins gp120 and gp41, which are expressed as a noncovalent, oligomeric complex on the surface of virus and virally infected cells. Entry of the virus into target cells proceeds through a cascade of events at the cell surface that include (1) binding of the viral surface glycoprotein gp120 to a cell surface receptor, (2) Env binding to fusion coreceptors, and (3) multiple conformational changes in gp41. The first high-affinity interaction between the virion and the cell surface is the binding of gp120 to cell surface CD4, which is the primary receptor for HIV-1 (Dalgleish et al.; 1984; Klatzmann et al., 1984; Maddon et al., 1986; McDougal et al., 1986). This binding induces conformational changes in gp120, which enable it to interact with one of several chemokine receptors (Berger, 1997; Bieniasz et al., 1998; Dragic et al., 1997; Littman, 1998). The CC-chemokine receptor 5 (CCR5) is the major co-receptor for macrophage-tropic (R5) strains, and plays a crucial role in the transmission of HIV-1 (Berger, 1997; Bieniasz et al., 1998; Dragic et al., 1997; Littman, 1998). T cell line-tropic (X4) viruses use CXCR4 to enter target cells, and usually, but not always, emerge late in disease progression or as a consequence of virus propagation in tissue culture. Some primary HIV-1 isolates are dual-tropic (R5X4) since they can use both co-receptors, though not always with the same efficiency (Connor et al., 1997; Simmons et al., 1996). Binding of gp120 to a chemokine receptor in turn triggers conformational changes in the viral transmembrane glycoprotein gp41, which mediates fusion of the viral and cellular membranes. Each stage of this multi-step process can be blocked with inhibitors of the appropriate viral or cellular protein, and the inhibitors of gp120, gp41, CD4 and coreceptor are collectively known as entry inhibitors. Entry inhibitors represent at least 4 distinct classes of agents based on their molecular targets and determinants of viral resistance (Olson and Maddon, 2003). Table 1 lists HIV-1 entry inhibitors known to be in clinical development or approved for clinical use.

PRO 542 is a tetravalent, third-generation CD4-IgG2 fusion protein comprising the D1D2 domains of CD4 genetically fused to the heavy and light chain constant regions of human IgG2 (Allaway et al., 1995; Zhu et al., 2001). This agent binds the HIV-1 envelope glycoprotein gp120 with nanomolar affinity and may inhibit virus attachment both by receptor blockade and by detaching gp120 from the virion surface, thereby irreversibly inactivating the virus.

TABLE 1
HIV-1 entry inhibitors

| Compound | Molecular Class | Target | Stage of Entry | Developer |
|---|---|---|---|---|
| PRO542 | CD4-Ig Fusion Protein | gp120 | Attachment | Progenics |
| BMS-488043 | Small Molecule | gp120 | Attachment | Bristol-Myers Squibb |
| TNX-355 | Humanized antibody | CD4 | Post-Attachment | Tanox |
| PRO 140 | Humanized antibody | CCR5 | Coreceptor | Progenics |
| CCR5mAb004 | Human antibody | CCR5 | Coreceptor | Human Genome |
| | | | | Sciences |
| SCH-D | Small Molecule | CCR5 | Coreceptor | Schering-Plough |
| (vicriviroc) | | | | |
| UK-427,857 | Small Molecule | CCR5 | Coreceptor | Pfizer |
| (maraviroc) | | | | |
| GW873140 | Small Molecule | CCR5 | Coreceptor | GlaxoSmithKline |
| TAK-652 | Small Molecule | CCR5 | Coreceptor | Takeda |
| AMD070 | Small Molecule | CXCR4 | Coreceptor | AnorMed |
| T- | Peptide | gp41 | gp41 Fusion | Trimeris/Roche |
| 20 (enfuvirtide) | | | | |

BMS-488043 is an optimized analog of BMS-378806 (see PCT International Publication Nos. WO 01/62255 A1 and WO 03/082289 A1), which has been variously reported to block gp120 attachment to CD4 (Lin et al., 2002; 2003) and post-attachment events (Si et al., 2004).
TNX-355 is a humanized IgG4 version of the anti-CD4 monoclonal antibody (mAb) 5A8, which blocks fusion events that occur post-attachment of gp120 to CD4 (Burkly et al., 1992; Moore et al., 1992).
PRO 140, a humanized anti-CCR5 mAb, and the small-molecule CCR5 antagonists, SCH-D (also now designated SCH 417670 or vicriviroc), UK-427,857 (also designated maraviroc) and GW873140, are discussed below.
CCR5mAb004 is a fully human mAb, generated using the Abgenix XenoMouse ® technology, that specifically recognizes and binds to CCR5 (Roschke et al., 2004). CCR5mAb004 has been reported to inhibit CCR5-dependent entry of HIV-1 viruses into human cells, and recently entered Phase 1 clinical trials (HGS Press Release, 2005).
The first small-molecule anti-CCR5 antagonist identified as capable of inhibiting HIV-I infection was TAK-779 (Baba et al., 1999). However, TAK-779 exhibited poor oral bioavailability (Baba et al., 2005) and local injection site irritation (Iizawa et al., 2003), and has been replaced in clinical development by a TAK-779 derivative, TAK-652 (Baba et al., 2005). TAK-652 is an orally bioavailable CCR5 antagonist with potent anti-HIV-1 activity in the nanomolar range in vitro and promising pharmacological profiles in vivo (Baba et al., 2005).
AMD070 is a second-generation CXCR4 inhibitor; the first-generation CXCR4 inhibitor AMD3100 did not demonstrate a favorable safety window for HIV-1 therapy (Schols et al., 2002).
Finally, T-20 was approved for salvage therapy of HIV-1 infection following favorable antiviral and safety profiles in each of two pivotal Phase 3 studies (Lalezari et al., 2003; Lazzarin et al., 2003).

### CCR5 as a Target for Anti-HIV-1 Therapy

As first demonstrated in 1986, HIV-1 binds to target cells via the CD4 receptor but requires additional host cell factors to mediate entry (Maddon et al., 1986). Over the next decade, a number of candidate coreceptors were proposed, but none reproducibly mediated viral entry when coexpressed with CD4 in otherwise nonpermissive cells. However, in 1996, certain chemokine receptors, mainly CCR5 and CXCR4, were shown to serve as requisite fusion coreceptors for HIV-1.

Cocchi et al. (1995) provided the first link between HIV-1 and chemokines, which are small (˜8 kDa) homologous soluble proteins. Chemokines mediate the recruitment and activation of immune cells. They are classified as CC-, CXC-, CX₃C- and XC-chemokines based on the number and sequential relationship of the first two of four conserved cysteine residues; most are either CC- or CXC-chemokines. The CC-chemokines RANTES, MIP-1α and MIP-1β, were shown to block replication of primary macrophage-tropic strains of HIV-1 (Cocchi et al., 1995). Using expression cloning techniques, Feng et al. (1996) discovered that the chemokine receptor fusin (later renamed CXCR4) was a fusion coreceptor for strains of HIV-1 adapted to growth on T cell lines. Shortly thereafter, several groups reported the cloning of CCR5, a CC chemokine receptor with specificity for RANTES, MIP-1α and MIP-1β (Combadiere et al., 1996; Raport et al., 1996; Samson et al., 1997), and others then demonstrated that CCR5 was the main entry cofactor used by primary macrophage-tropic HIV-1 isolates (Alkhatib et al., 1996; Choe et al., 1996; Deng et al., 1996; Doranz et al., 1996; Dragic et al., 1996). The patterns of CCR5 and CXCR4 expression helped solve long-standing riddles concerning the tropism of different strains of HIV-1. Macrophage-tropic, T-cell-line-tropic and dual-tropic viruses could be more descriptively classified as being R5, X4 and R5X4 viruses based on their abilities to utilize CCR5, CXCR4 or both receptors, respectively, for entry.

A variety of other chemokine receptors can function as HIV-1 coreceptors when over-expressed in vitro. The list includes CCR8, Apj, V28, US28, CCR2b, CCR3, gprl, Bonzo (STRL33, TYMSTR), and BOB (gprl5). Clearly, proteins belonging to the chemokine receptor family have biochemical properties that promote HIV-1 membrane fusion. However, most of the above-mentioned coreceptors are not very efficient, are not normally coexpressed with CD4, and function only with certain strains of HIV-1, HIV-2 or SIV. The in vivo relevance of these alternative coreceptors has not been established.

Several factors make CCR5 an attractive target for new antiretroviral therapies. CCR5 plays a central role in HIV-1 transmission and pathogenesis, and naturally-occurring mutations in CCR5 confer protection from HIV-1 infection and disease progression. The most notable CCR5 polymorphism involves a 32 bp deletion in the coding region of CCR5 (A32) (Liu et al., 1996). The A32 allele encodes a nonfunctional receptor that fails to reach the cell surface. Individuals who possess one normal and one mutant CCR5 gene express lower levels of CCR5, and their T cells are less susceptible to R5 virus infection in vitro (Liu et al., 1996; Wu et al., 1997). A32 heterozygotes experience a milder course of disease characterized by reduced viral burdens and delayed progression to AIDS (Huang et al., 1996; Michael et al., 1997). These results support the concept that reducing CCR5 availability can lower viral replication and slow disease progression. Individuals with two mutant CCR5 genes comprise a significant fraction of people of northern European descent; the demography is suggestive of a prior pandemic of a CCR5-using pathogen. Such individuals represent human CCR5 “knockouts” in that they do not express a functional CCR5 protein. Except in rare instances (Balotta et al., 1997; Biti et al., 1997; O'Brien et al., 1997), these individuals are resistant to HIV-1 infection (Huang et al., 1996; Liu et al., 1996; Michael et al., 1997; Samson et al., 1997), and their T cells cannot be infected with R5 viruses in vitro (Liu et al., 1996). These findings underscore the central role of CCR5 in HIV-1 transmission. In fact, it is now known that R5 viruses mediate transmission in nearly all cases and mediate progression to AIDS in most cases.

Importantly, individuals who lack CCR5 enjoy normal health and display no obvious immunologic or other defects. This may reflect the redundancy of chemokine signaling pathways and the rather limited pattern of expression of CCR5. CCR5 expression is largely confined to activated T cells and macrophages, which represent the primary targets for HIV-1 infection in vivo, although low-level CCR5 expression has been reported on other tissues, such as smooth muscle (Schecter et al., 2000).

CCR5 knockout mice have been generated and provide further insight into the effects of abrogating CCR5 function. CCR5 knockout mice develop normally and are ostensibly healthy, although minor alterations in immune responses can be observed upon challenge with particular pathogens (Huffnagle et al., 1999; Schuh et al., 2002; Tran et al., 2000; Zhou et al., 1998). In contrast, the CXCR4 knockout is a lethal phenotype in mice (Lapidot et al., 2001), and has not been observed in humans.

Taken together, these genetic analyses strongly support a new therapeutic approach based on CCR5 as a drug target. The error-prone nature of reverse transcriptase generates immense genetic diversity that fosters the development of drug-resistant isolates, and HIV-1's ability to utilize multiple fusion coreceptors provides one path to resistance. Drug-resistant viruses have been isolated for all marketed antiretrovirals, which nevertheless provide important therapeutic benefit when used in appropriate combinations. Thus, despite the potential emergence of drug-resistant viruses, CCR5-targeting agents may serve as a new treatment paradigm for HIV-1 infection.

Although the apparent non-essential nature of CCR5 suggests that CCR5 antagonists may be well tolerated in vivo, further studies are required to determine that long-term effects of abrogating CCR5 function in individuals whose immune systems developed in its presence. Such potentially deleterious effects may be mitigated by use of agents that bind to CCR5 and inhibit binding of HIV-1 thereto, but do not impair normal CCR5 function. One agent demonstrated to have such properties is the humanized anti-CCR5 mAb, PRO 140, which effectively blocks HIV-1 replication at concentrations that do not inhibit the physiologic activity of CCR5 (Olson et al., 1999). PRO 140 was identified using a fluorescence resonance energy transfer (RET) assay screen for anti-HIV activity. It is potently antiviral, having an IC₉₀ of about 4 μg/ml (Olson et al., 1999; Trkola et al., 2001) and protects diverse primary target cell types (Ketas et al., 2003; Olson and Maddon, 2003). Repeated administration of PRO 140 led to prolonged control of HIV-1 replication without viral escape in the hu-PBL SCID mouse model, and PRO 140 is currently in Phase 1 human clinical trials.

Subsequent to the identification of the small-molecule CCR5 antagonist, TAK-779 (Baba et al., 1999), several other small-molecule CCR5 antagonists have been identified. Four of these (SCH-C, SCH-D, UK-427,857, GW873140) have completed similarly designed Phase 1 studies in HIV-infected individuals (Reynes et al., 2002; Schurmann et al., 2004; Dorr et al., 2003; Lalezari et al., 2004). Each of these agents mediated dose-dependent ˜1 log₁₀ mean reductions in HIV-1 RNA levels during the treatment period of 10-14 days. As expected, viral loads rebounded to baseline levels following cessation of therapy. The most common drug-related side-effects were neurologic (headache, dizziness) and gastrointestinal (nausea, diarrhea, flatulence), and these were not dose limiting. With the exception of SCH-C (Reyes et al., 2001), none of the above-identified agents induced clinically significant changes in QTc intervals.

A double-blind, placebo-controlled, single oral dose study has also been conducted to evaluate the safety, tolerability, and pharmacokinetics of TAK-652, the successor compound to TAK-779, in healthy male volunteers (Baba et al., 2005). The single administration of TAK-652 solution was reportedly safe and well tolerated (Baba et al., 2005).

Overall, these studies provide preliminary validation of CCR5 as a target for HIV-1 therapy. While the small-molecule CCR5 antagonists represent patentably distinct chemical series with differing pharmacokinetic and metabolic properties, the compounds share many properties in their inhibition of CCR5 function, binding site on CCR5, resistance profiles, and dosing regimen. These similarities may conceivably limit the number of genuine treatment options afforded by small-molecule CCR5 antagonists. Moreover, it remains to be determined whether there are untoward consequences of chronic blockade of CCR5 function, and the utility of small-molecule CCR5 antagonists for HIV-1 therapy remains to be established by demonstration of appropriate safety and efficacy in Phase 3 clinical studies.

### Monoclonal Antibody Therapeutics

In recent years, mAb products have provided new standards of care in diverse disease settings. Currently, 18 mAbs are approved by the U.S. Food and Drug Administration (FDA) for indications including cancer, autoimmune disease, transplant rejection and viral infection. Notably, 14 mAbs have been approved since 2000. In many instances, mAbs provide safety, efficacy and ease-of-use profiles that are unrivalled by small-molecule compounds. Examples include Synagis (MedImmune, Inc., Gaithersburg, Md.), a humanized mAb to respiratory syncytial virus (RSV), and Rituxan (Genentech, San Francisco, Calif.), an anti-CD20 mAb that provides the standard of care for non-Hodgkin's lymphoma.

The humanized anti-CCR5 mAb, PRO 140, is structurally, functionally and mechanistically distinct from the small-molecule CCR5 antagonists and therefore represents a unique CCR5 inhibitor class. PRO 140 is a humanized version of the murine mAb, PA14, which was generated against CD4⁺CCR5⁺ cells (Olson et al., 1999). PRO 140 binds to CCR5 expressed on the surface of a cell, and potently inhibits HIV-1 entry and replication at concentrations that do not affect CCR5 chemokine receptor activity in vitro and in the hu-PBL-SCID mouse model of HIV-1 infection (Olson et al., 1999; Trkola et al., 2001). The latter finding provides in vivo proof-of-concept for PRO 140 anti-HIV-1 therapy, and PRO 140 is currently undergoing Phase 1a clinical studies.

Important differences between PRO 140 and small-molecule CCR5 antagonists are summarized in Table 2. It is evident from Table 2 that, whereas small-molecule CCR5 antagonists in development share many properties, PRO 140 is clearly distinct from these small-molecule inhibitors. The differences between the two CCR5 inhibitor classes reveal that PRO 140 may offer a fundamentally distinct, and in many ways complementary, product profile from that of small-molecule CCR5 antagonists. Indeed, PRO 140 represents a novel therapeutic approach to treating HIV-1 infection and could play an important role in HIV-1 therapy irrespective of whether small-molecule CCR5 antagonists are ultimately clinically approved.

### Synergistic Inhibition of HIV-1 Infection by Different Classes of Inhibitors

Synergistic inhibition of HIV-1 entry has previously been demonstrated using certain anti-Env antibodies in combination with other anti-Env antibodies (Thali et al., 1992; Tilley et al., 1992; Laal et al., 1994; Vijh-Warrier et al., 1996; Li et al., 1997; Li et al., 1998), anti-CD4 antibodies (Burkly et al., 1995), or CD4-based proteins (Allaway et al., 1993). Similarly, synergies have been observed using anti-CCR5 antibodies in combination with other anti-CCR5 antibodies, CC-chemokines, or CD4-based proteins (Olson et al., 1999). Prior studies described in PCT International Publication No. WO 00/35409, published Jun. 22, 2000, examined combinations of HIV-1 attachment inhibitors and CCR5 coreceptor inhibitors. Prior studies described in PCT International Publication No. WO 01/55439, published Aug. 2, 2001, examined combinations of inhibitors of gp41 fusion intermediates and HIV-1 attachment. Prior studies described in PCT International Publication No. WO 02/22077, published Mar. 21, 2002, examined combinations of fusion inhibitors and CCR5 coreceptor inhibitors, as well as the triple combination of fusion inhibitors, CCR5 coreceptor inhibitors and HIV-1 attachment inhibitors. However, no prior study has examined the combination of different classes of CCR5 coreceptor inhibitors, such as anti-CCR5 mAbs and non-antibody CCR5 antagonists.

TABLE 2
Comparison of PRO 140 and small-molecule CCR5
antagonists under development

| | Small Molecules | PRO 140 |
|---|---|---|
| Identification Screen | Chemokine Binding | HIV-1 Entry |
| Block Natural Activity of CCR5 | Yes | No |
| Potential for Immune | Yes | No |
| Suppression/Dysregulation | | |
| Tolerability | Cardiac, Neurological | No Toxicity |
| | Toxicities for some | |
| Binding site on CCR5 | Common Hydrophobic | Extracellular Epitope that |
| | Pocket defined by | spans Multiple Hydrophilic |
| | Transmembrane Regions of | Domains |
| | CCR5 | |
| Viral Cross-Resistance | Significant | Limited |
| Development of Resistance In Vitro | 6 to 19 weeks | None at 40 weeks |
| Drug-Drug Interactions | Significant | Unlikely |
| Food Interactions | Significant | Unlikely |
| Dosing | Once or Twice Daily | Biweekly to Monthly |

### SUMMARY OF THE INVENTION

This invention provides a method of inhibiting HIV-2 infection of a susceptible cell by HIV-2 which comprises subjecting the susceptible cell to an effective HIV-2 infection inhibiting dose of (a) a humanized antibody designated PRO 140, or of (b) an anti-CCR5 receptor monoclonal antibody which (i) binds to CD4+CCR5+ cells and inhibits fusion of HIV-1 with such cells, (ii) inhibits HIV-1 fusion with CD4+CCR5+ cells with a potency equal or greater than that of PRO 140, (iii) coats CD4+CCR5+ cells in the subject without reducing the number of such cells in the subject, and/or (iv) binds to the subject's CD4+CCR5+ cells without inducing an increase in the subject's plasma concentration of circulating β-chemokines, wherein PRO 140 comprises (i) two light chains, each light chain comprising the light chain variable (V_{L}) and constant (C_{L}) regions encoded by the plasmid designated pVK:HuPRO140-VK (ATCC Deposit Designation PTA-4097), and (ii) two heavy chains, each heavy chain comprising the heavy chain variable (V_{H}) and constant (C_{H}) regions encoded either by the plasmid designated pVg4:HuPRO140 HG2-VH (ATCC Deposit Designation PTA4098) or by the plasmid designated pVg4:HuPRO140 (mut B+D+I)-VH (ATCC Deposit Designation PTA-4099), wherein the effective HIV-2 infection inhibiting dose comprises from 0.1 mg per kg to 25 mg per kg of the subject's body weight, so as to thereby inhibit the infection of the susceptible cell by HIV-2.

This invention also provides a method of inhibiting HIV-2 infection in an HIV-2-infected human subject which comprises administering to the subject an effective HIV-2 infection inhibiting dose of (a) a humanized antibody designated PRO 140, or of (b) an anti-CCR5 receptor monoclonal antibody which (i) binds to CD4+CCR5+ cells in the subject and inhibits fusion of HIV-1 with such cells, (ii) inhibits HIV-1 fusion with CD4+CCR5+ cells with a potency equal or greater than that of PRO 140, (iii) coats CD4+CCR5+ cells in the subject without reducing the number of such cells in the subject, and/or (iv) binds to the subject's CD4+CCR5+ cells without inducing an increase in the subject's plasma concentration of circulating β-chemokines, wherein PRO 140 comprises (i) two light chains, each light chain comprising the light chain variable (V_{L}) and constant (C_{L}) regions encoded by the plasmid designated pVK:HuPRO140-VK (ATCC Deposit Designation PTA4097), and (ii) two heavy chains, each heavy chain comprising the heavy chain variable (V_{H}) and constant (C_{H}) regions encoded either by the plasmid designated pVg4:HuPRO140 HG2-VH (ATCC Deposit Designation PTA4098) or by the plasmid designated pVg4:HuPRO140 (mut B+D+I)-VH (ATCC Deposit Designation PTA-4099), wherein the effective HIV-2 infection inhibiting dose comprises from 0.1 mg per kg to 25 mg per kg of the subject's body weight, so as to thereby inhibit HIV-2 infection in the subject.

This invention also provides a method of inhibiting in a human subject the onset or progression of an HIV-2-associated disorder, the inhibition of which is effected by inhibiting fusion of HIV-2 which comprises administering to the subject at a predefined interval effective fusion-inhibitory doses of a humanized antibody designated PRO 140, or of an anti-CCR5 receptor antibody which (i) binds to CD4+CCR5+ cells in the subject and inhibits fusion of HIV-1 with such cells, (ii) inhibits HIV-1 fusion with the subject's CD4+CCR5+ cells with a potency characterized by an IC90 of 10 μg/ml or less, (iii) coats the subject's CD4+CCR5+ cells without reducing the number of such cells in the subject, and/or (iv) binds to the subject's CD4+CCR5+ cells without inducing an increase in the subject's plasma concentration of circulating β-chemokines, wherein PRO 140 comprises (i) two light chains, each light chain comprising the light chain variable (V_{L}) and constant (C_{L}) regions encoded by the plasmid designated pVK:HuPRO140-VK (ATCC Deposit Designation PTA4097), and (ii) two heavy chains, each heavy chain comprising the heavy chain variable (V_{H}) and constant (C_{H}) regions encoded either by the plasmid designated pVg4:HuPRO140 HG2-VH (ATCC Deposit Designation PTA-4098) or by the plasmid designated pVg4:HuPRO140 (mut B+D+I)-VH (ATCC Deposit Designation PTA-4099), wherein each administration of the antibody delivers to the subject from 0.1 mg per kg to 25 mg per kg of the subject's body weight, so as to thereby inhibit the onset or progression of the HIV-2-associated disorder in the subject.

This invention further provides a method of reducing the likelihood of a human subject's contracting infection by HIV-2 which comprises administering to the subject at a predefined interval effective fusion-inhibitory doses of a humanized antibody designated PRO 140, or of an anti-CCR5 receptor antibody which (i) binds to CD4+CCR5+ cells in the subject and inhibits fusion of HIV-1 with such cells, (ii) inhibits HIV-1 fusion with the subject's CD4+CCR5+ cells with a potency characterized by an IC90 of 10 μg/ml or less, (iii) coats the subject's CD4+CCR5+ cells without reducing the number of such cells in the subject, and/or (iv) binds to the subject's CD4+CCR5+ cells without inducing an increase in the subject's plasma concentration of circulating β-chemokines, wherein PRO 140 comprises (i) two light chains, each light chain comprising the light chain variable (V_{L}) and constant (C_{L}) regions encoded by the plasmid designated pVK:HuPRO140-VK (ATCC Deposit Designation PTA4097), and (ii) two heavy chains, each heavy chain comprising the heavy chain variable (V_{H}) and constant (C_{H}) regions encoded either by the plasmid designated pVg4:HuPRO140 HG2-VH (ATCC Deposit Designation PTA-4098) or by the plasmid designated pVg4:HuPRO140 (mut B+D+I)-VH (ATCC Deposit Designation PTA4099), wherein each administration of the antibody delivers to the subject from 0.1 mg per kg to 25 mg per kg of the subject's body weight, so as to thereby reduce the likelihood of the subject's contracting an HIV-2 infection.

### BRIEF DESCRIPTION OF THE FIGURES

FIG. 1

Humanized PRO 140 is potently antiviral. The in vitro neutralization activity of murine and humanized PRO 140 was tested against four primary R5 HIV-1 isolates using a whole virus replication assay. The data reflect the median values from 8 or more independent assays. The genetic subtypes of the viruses are indicated in parentheses.

FIG. 2

Antiviral activity is independent of target cell. Inhibition of infection of four different target cells by three primary R5 HIV-1 isolates with was tested.

FIG. 3

In vitro HIV-1 susceptibility to PRO 140 quantified using the PhenoSense™ entry assay. PRO 140 was tested for activity against 20 primary HIV-1 isolates in the PhenoSense HIV Entry™ assay at ViroLogic, Inc. (now Monogram Biosciences, South San Francisco, Calif.). Drug susceptibility is reported as IC₅₀ values, which represent the concentration required for 50% inhibition of viral infectivity.

FIG. 4

PRO 140 blocks HIV-1 but not chemokine signaling. The effects of PRO 140 on the inhibition of RANTES-induced calcium mobilization in L1.2-CCR5 cells and on inhibition of HIV-1_{JR-FL} replication in PBMC cultures were determined. Similar results were obtained for MIP-1α and MIP-1β.

FIG. 5

PRO 140 provides prolonged control of viral replication in HIV-1-infected mice. SCID mice were reconstituted with normal human peripheral blood mononuclear cells and infected 2 weeks later with HIV-1_{JR-CSF}. Multiple doses of PRO 140 were administered following attainment of steady state viral levels. Plasma viral loads pre- and post-injection are indicated.

FIG. 6

PRO 140 coats but does not deplete CCR5 lymphocytes. Healthy male volunteers (n=4) were treated with a single intravenous infusion of PRO 140 at a dose level of 2 mg/kg. At the indicated times post-treatment, blood was collected and analyzed for CCR5 lymphocyte levels. The group mean values and standard deviations are indicated.

FIG. 7

Serum concentrations of PRO 140. Healthy male volunteers were treated with a single intravenous infusion of PRO 140 at dose levels of 0.1, 0.5 and 2.0 mg/kg, as indicated. At the indicated times post-treatment, serum was collected, cryopreserved, and analyzed for PRO 140 levels. Data for individual patients are indicated.

FIG. 8

PRO 140 does not affect plasma chemokine levels. Healthy male volunteers were treated with a single intravenous infusion of 0.1 mg/kg PRO 140 (Cohort 1), 0.5 mg/kg PRO 140 (Cohort 2) or matched placebo. At the indicated times post-treatment, plasma was collected, cryopreserved and analyzed for levels of RANTES. The Lower Limit of Quantification of the assay was 415 pg RANTES/mL plasma. Data represent the group mean values.

FIG. 9

Synergistic inhibition of HIV-1 fusion exhibited by PRO 140 with different compounds. Interactions between PRO 140 and small-molecule, peptide, mAb, and chimeric CD4-immunoglobulin inhibitors of CCR5, CD4, gp120 and gp41 targets for inhibiting HIV-1 fusion were assessed using the RET assay. Mean combination index (CI) values with 95% confidence intervals are plotted for data obtained using the compounds combined in a 1:1 molar ratio. A CI value of <1 indicates synergistic interactions; a CI value of 1 indicates additive interactions; and a CI value of >1 indicates antagonistic interactions.

FIG. 10

PRO 140 coats but does not deplete lymphocytes. Healthy male volunteers (n=4) were treated with a single intravenous infusion of PRO 140 at a dose level of 5 mg/kg. At the indicated times post-treatment, blood was collected and analyzed for CCR5 lymphocyte levels. The group mean values and standard deviations are indicated.

FIG. 11

Dose-response curves for inhibition of HIV-1_{JR-FL} envelope-mediated membrane fusion by combinations of CCR5 inhibitors. Dilutions were analyzed in triplicate wells, and the data points depict the mean and standard deviations of replicates. (A) PRO 140 and UK-427,857 were tested individually and in a 1:1 fixed molar ratio over the indicated range of concentrations. In the experiment depicted, IC50 and IC90 values were 2.9 nM and 11 nM for PRO 140, 5.0 nM and 21 nM for UK-427,857, and 2.1 nM and 4.6 nM for the combination. CI50 and CI90 values were 0.58 and 0.32, respectively. (B) SCH-D and UK-427,857 were tested individually and in a 1:1 fixed molar ratio over the indicated range of concentrations. In the experiment depicted, IC50 and IC90 values were 5.5 nM and 34 nM for SCH-D, 9.7 nM and 59 nM for UK-427,857, and 6.1 nM and 31 nM for the combination. CI50 and CI90 values were 0.87 and 0.73, respectively.

FIG. 12

Inhibition of PRO 140-PE binding to CEM.NKR-CCR5 cells by unlabeled PRO 140, UK-427,857 and SCH-D. CEM.NKR-CCR5 cells were incubated with varying concentrations of unlabeled PRO 140, UK-427,857 or SCH-D for 30 min at room temperature in PBSA buffer prior to addition of 5 nM PRO 140-PE for an additional 30 min. Cells were washed and then analyzed by flow cytometry for both the mean fluorescence intensity (MFI) of binding and the percent of cells gated for positive binding of PRO 140-PE. Inhibition was assessed on the basis of both MFI (A) and percent cells gated (B).

FIG. 13

Inhibition of ³H-UK-427,857 binding by unlabeled UK-427,857, SCH-D and PRO 140. (A) CEM.NKR-CCR5 cells were pre-incubated with varying concentrations of unlabeled UK-427,857, SCH-D or PRO 140 for 30 min in PBSA buffer at ambient temperature prior to the addition of at 2 nM ³H-UK-427,857 for an additional 30 min. Cells were washed and then analyzed for radioactivity by scintillation counting. (B) The stability of UK-427,857 binding under the assay conditions was examined by pre-incubating CEM.NKR-CCR5 cells with 2 nM ³H-UK-427,857 prior to washing, addition of unlabeled compounds for 30 min, and processing as described above.

FIG. 14

Table showing results of Phase 1b Study.

FIG. 15

Graph depicting change in viral load from baseline in Phase 1b Study.

FIGS. 16A and 16B:

Graphs depicting change in CD4+ cell counts in Phase 1b Study.

FIG. 17: Graph depicting mean log₁₀ in HIV-1 RNA change from baseline in Phase 1b Study.

FIG. 18: Graph depicting mean log₁₀ change in HIV RNA, day 10 results and individual subject nadirs.

FIG. 19: Graph depicting virological response rate determined at the completion of the study. Percent of subjects in study cohorts with ≧1 log₁₀ reduction in HIV-1 RNA.

FIG. 20: Coreceptor virus tropism (Trofile™, Monogram Biosciences).

FIG. 21: Median PRO 140 and PA14 EC₅₀ (ug/ml) values for HIV-2 isolates. Each of the five HIV-2 R5 isolates was susceptible to inhibition by both PRO 140 and PA14. Neither PRO 140 nor PA14 had any measurable effect on replication of R5X4 HIV-2 isolates. EC₅₀ values for HIV-1 were similar to those for HIV-2 (0.09 to 0.46 ug/ml for HIV-1; 0.07 to 7.6 ug/ml for HIV-2). The EC₅₀ values observed for PRO 140 and PA14 were not significantly different in this study (p=0.11, two-tailed Wilcoxon signed rank test). The median maximum percent inhibition (MPI) values were >94% for each of the R5 HIV-1 and HIV-2 isolates tested in this study.

FIGS. 22A, 22B, and 22C: Inhibition data were analyzed using GraphPad Prism software (Graphpad Software, San Diego, Calif.)

A. Graph depicting results of PRO 140 vs R5 HIV-1 inhibition assay. B. Graph depicting results of PRO 140 vs R5 HIV-2 inhibition assay. C. Graph depicting results of PRO 140 vs R5X4 HIV-2 inhibition assay.

### DETAILED DESCRIPTION OF THE INVENTION

As used in this application, except as otherwise expressly provided herein, each of the following terms shall have the meaning set forth below.

“Administering” refers to delivering in a manner which is effected or performed using any of the various methods and delivery systems known to those skilled in the art. Administering can be performed, for example, topically, intravascularly, intravenously, pericardially, orally, parenterally, via implant, transmucosally, dermally, transdermally, intradermally, intramuscularly, subcutaneously, intraperitoneally, intrathecally, intralymphatically, intralesionally, epidurally, rectally, intravaginally, intraocularly, intrasinally, nasally, intraspinally, mucosally, transmucosally, transplacentally or by in vivo electroporation. An agent or composition may also be administered in an aerosol, such as for pulmonary and/or intranasal delivery. Administering can also be performed, for example, once, a plurality of times, and/or over one or more extended periods.

An “antibody” shall include, without limitation, an immunoglobulin molecule comprising two heavy chains and two light chains and which recognizes an antigen. The immunoglobulin molecule may derive from any of the commonly known classes, including but not limited to IgA, secretory IgA, IgG and IgM. IgG subclasses are also well known to those in the art and include but are not limited to human IgG1, IgG2, IgG3 and IgG4. “Antibody” includes, by way of example, both naturally occurring and non-naturally occurring antibodies; monoclonal and polyclonal antibodies; chimeric and humanized antibodies; human or nonhuman antibodies; wholly synthetic antibodies; and single chain antibodies. A nonhuman antibody may be humanized by recombinant methods to reduce its immunogenicity in man. Methods for humanizing antibodies are well known to those skilled in the art. “Antibody” also includes, without limitation, a fragment or portion of any of the afore-mentioned immunoglobulin molecules and includes a monovalent and a divalent fragment or portion. Antibody fragments include, for example, Fc fragments and antigen-binding fragments (Fab).

An “anti-chemokine receptor antibody” refers to an antibody which recognizes and binds to an epitope on a chemokine receptor. As used herein, “anti-CCR5 antibody” refers to an antibody which recognizes and binds to an epitope on the CCR5 chemokine receptor.

“Attachment” means the process that is mediated by the binding of the HIV-1 envelope glycoprotein to the human CD4 receptor, which is not a fusion coreceptor.

As used herein, “CCR5” or “R5”, is a chemokine receptor which binds members of the C—C group of chemokines and whose amino acid sequence comprises that provided in Genbank Accession Number 1705896 and related polymorphic variants. As used herein, CCR5 includes, without limitation, extracellular portions of CCR5 capable of binding the HIV-1 envelope protein. “CCR5” and “CCR5 receptor” are used synonymously.

“CD4” means the mature, native, membrane-bound CD4 protein comprising a cytoplasmic domain, a hydrophobic transmembrane domain, and an extracellular domain which binds to the HIV-1 gp120 envelope glycoprotein.

“CDR”, or complementarity determining region, means a highly variable sequence of amino acids in the variable domain of an antibody.

A “cell” includes a biological cell, e.g., a HeLa cell, a lymphocyte, a PBMN cell, and a non-biological cell, e.g., a phospholipid vesicle or virion. A “cell susceptible to HIV infection” may also be referred to as a “target cell” and includes a cell capable of being infected by or fusing with HIV or an HIV-infected cell.

“CXCR4” or “R4” is a chemokine receptor which binds members of the C—X—C group of chemokines and whose amino acid sequence comprises that provided in Genbank Accession No 400654 and related polymorphic variants. As used herein, CXCR4 includes extracellular portions of CXCR4 capable of binding the HIV-1 envelope protein.

“Exposed” to HIV-1 refers to contact with HIV-1 such that infection could result.

A “fully human” antibody refers to an antibody wherein all of the amino acids correspond to amino acids in human immunoglobulin molecules. “Fully human” and “human” are used synonymously.

“HIV” refers to the human immunodeficiency virus. HIV shall include, without limitation, HIV-1. HIV-1 includes but is not limited to extracellular virus particles and the forms of HIV-1 associated with HIV-1 infected cells. The human immunodeficiency virus (HIV) may be either of the two known types of HIV (HIV-1 or HIV-2). The HIV-1 virus may represent any of the known major subtypes (classes A, B, C, D, E, F, G, H, or J), outlying subtype (Group 0), or an as yet to be determined subtype of HIV-1. HIV-1_{JR-FL} is a strain that was originally isolated at autopsy from the brain tissue of an AIDS patient. The virus has been cloned and the DNA sequences of its envelope glycoproteins are known (GenBank Accession No. U63632). In terms of sensitivity to inhibitors of viral entry, HIV-1_{JR-FL} is known to be highly representative of primary HIV-1 isolates. “JRCSF” refers to a HIV-1 isolate of subtype B. JRCSF is a strain originally isolated from cerebral spinal fluid and brain tissue of an AIDS patient (Science 236, 819-822, 1987). The virus has been cloned and its genome DNA sequence is known (GenBank Accession No. M38429). Unlike HIV isolate JRFL, JRCSF does not productively infect macrophages.

A “humanized” antibody refers to an antibody wherein some, most or all of the amino acids outside the CDR regions are replaced with corresponding amino acids derived from human immunoglobulin molecules. In one embodiment of the humanized forms of the antibodies, some, most or all of the amino acids outside the CDR regions have been replaced with amino acids from human immunoglobulin molecules, whereas some, most or all amino acids within one or more CDR regions are unchanged. Small additions, deletions, insertions, substitutions or modifications of amino acids are permissible as long as they do not abrogate the ability of the antibody to bind a given antigen. Suitable human immunoglobulin molecules include IgG1, IgG2, IgG3, IgG4, IgA, IgE and IgM molecules. A “humanized” antibody retains an antigenic specificity similar to that of the original antibody.

One skilled in the art would know how to make the humanized antibodies of the subject invention. Various publications, several of which are hereby incorporated by reference into this application, also describe how to make humanized antibodies. For example, the methods described in U.S. Pat. No. 4,816,567 comprise the production of chimeric antibodies having a variable region of one antibody and a constant region of another antibody.

U.S. Pat. No. 5,225,539 describes another approach for the production of a humanized antibody. This patent describes the use of recombinant DNA technology to produce a humanized antibody wherein the CDRs of a variable region of one immunoglobulin are replaced with the CDRs from an immunoglobulin with a different specificity such that the humanized antibody would recognize the desired target but would not be recognized in a significant way by the human subject's immune system. Specifically, site-directed mutagenesis is used to graft the CDRs onto the framework. Other approaches for humanizing an antibody are described in U.S. Pat. Nos. 5,585,089 and 5,693,761, and PCT International Publication No. WO 90/07861, which describe methods for producing humanized immunoglobulins. These have one or more CDRs and possible additional amino acids from a donor immunoglobulin and a framework region from an accepting human immunoglobulin. These patents describe a method to increase the affinity of an antibody for the desired antigen. Some amino acids in the framework are chosen to be the same as the amino acids at those positions in the donor rather than in the acceptor. Specifically, these patents describe the preparation of a humanized antibody that binds to a receptor by combining the CDRs of a mouse monoclonal antibody with human immunoglobulin framework and constant regions. Human framework regions can be chosen to maximize homology with the mouse sequence. A computer model can be used to identify amino acids in the framework region which are likely to interact with the CDRs or the specific antigen and then mouse amino acids can be used at these positions to create the humanized antibody. The above methods are merely illustrative of some of the methods that one skilled in the art could employ to make humanized antibodies. Methods for making fully human antibodies are also well known to one skilled in the art. For example, fully human monoclonal antibodies can be prepared by immunizing animals transgenic for large portions of human immunoglobulin heavy and light chain loci. See, e.g., U.S. Pat. Nos. 5,591,669, 5,545,806, 5,545,807, 6,150,584, and references cited therein, the contents of which are incorporated herein by reference. These transgenic animals have been genetically modified such that there is a functional deletion in the production of endogenous (e.g., murine) antibodies. The animals are further modified to contain all or a portion of the human germ-line immunoglobulin gene locus such that immunization of these animals will result in the production of fully human antibodies to the antigen of interest. Following immunization of these animals (e.g., XenoMouse® (Abgenix), HuMAb-Mouse® (Medarex/GenPharm)), monoclonal antibodies can be prepared according to standard hybridoma technology. These monoclonal antibodies will have human immunoglobulin amino acid sequences and therefore will not provoke human anti-mouse antibody (HAMA) responses when administered to humans. In vitro methods also exist for producing human antibodies. These include phage display technology (U.S. Pat. Nos. 5,565,332 and 5,573,905) and in vitro stimulation of human B cells (U.S. Pat. Nos. 5,229,275 and 5,567,610). The contents of these patents are incorporated herein by reference.

Nucleic acids encoding heavy and light chains of the humanized PRO 140 antibody have been deposited with the ATCC. Specifically, the plasmids designated pVK-HuPRO140, pVg4-HuPRO140 (mut B+D+I) and pVg4-HuPRO140 HG2, respectively, were deposited pursuant to, and in satisfaction of, the requirements of the Budapest Treaty with the ATCC, Manassas, Va., U.S.A. 20108, on Feb. 22, 2002, under ATCC Accession Nos. PTA 4097, PTA 4099 and PTA 4098, respectively.

The half-life of the humanized PRO 140 antibody may be increased to prolong exposure of the drug following administration. For example, the half-life of PRO 140 in serum or plasma may be extended, and/or the amount and time that PRO 140 coats CCR5+ target cells may be extended. Illustrative methods include conjugation to polyethylene glycol (PEG), (pegylation), or monomethoxypolyethylene glycol (mPEG); and molecularly engineering PRO 140, e.g., by site directed mutagenesis, to have altered pH-dependent binding to the human neonatal Fc receptor (FcRn), an MHC class I-like Fc receptor. (See, e.g., S. B. Petkova et al., 2006, Int'l Immunol., 18(12):1759-1769; P. R. Hinton et al., 2006, J. Immunol., 176:346-356).

The production of antibody or antibody fragment-polymer conjugates having an effective size or molecular weight that confers an increase in serum half-life, an increase in mean residence time in circulation (MRT) and/or a decrease in serum clearance rate over underivatized antibody or antibody fragments. The antibody fragment-polymer conjugates can be made by derivatizing the desired antibody fragment with an inert polymer. It will be appreciated that any inert polymer which provides the conjugate with the desired apparent size, or which has the selected actual molecular weight, is suitable for use in constructing suitable antibody fragment-polymer conjugates. Many inert polymers are suitable for use in pharmaceuticals. See, e.g., Davis et al., Biomedical Polymers: Polymeric Materials and Pharmaceuticals for Biomedical Use, pp. 441-451 (1980). A non-proteinaceous polymer is particularly advantageous. The non-proteinaceous polymer ordinarily is a hydrophilic synthetic polymer, i.e., a polymer not otherwise found in nature. However, polymers which exist in nature and are produced by recombinant or in vitro methods are also useful, as are polymers which are isolated from native sources. Hydrophilic polyvinyl polymers, e.g., polyvinylalcohol and polyvinvypyrrolidone, are suitable. Particularly useful are polyalkylene ethers such as polyethylene glycol (PEG); polyoxyalklyenes such as polyoxyethylene, polyoxypropylene and block copolymers of polyoxyethylene and polyoxypropylene (Pluronics); polymethacrylates; carbomers; branched or unbranched polysaccharides which comprise the saccharide monomers D-mannose, D- and L-galactose, fucose, fructose, D-xylose, L-arabinose, D-glucuronic acid, sialic acid, D-galacturonic acid, D-mannuronic acid (e.g., polymannuronic acid, or alginic acid), D-glucosamine, D-galactosamine, D-glucose and neuraminic acid including homopolysaccharides and heteropolysaccharides such as lactose, amylopectin, starch, hydroxyethyl starch, amylose, dextran sulfate, dextran, dextrins, glycogen, or the polysaccharide subunit of acid mucopolysaccharides, e.g., hyaluronic acid, polymers of sugar alcohols such as polysorbitol and polymannitol, heparin or heparon. The polymer prior to cross-linking need not, but can be, water soluble, but the final conjugate needs to be water soluble. The conjugate exhibits a water solubility of at least about 0.01 mg/ml, or at least about 0.1 mg/ml, or at least about 1 mg/ml. In addition the polymer should not be highly immunogenic in the conjugate form, nor should it possess viscosity that is incompatible with intravenous infusion or injection if the conjugate is intended to be administered by such routes. In one embodiment, the polymer contains only a single group that is reactive. This helps to avoid cross-linking of protein molecules. However, reaction conditions can be maximized to reduce cross-linking, or to purify the reaction products through gel filtration or ion-exchange chromatography to recover substantially homogeneous derivatives. In other embodiments, the polymer contains two or more reactive groups for the purpose of linking multiple antibody fragments to the polymer backbone. Again, gel filtration or ion-exchange chromatography can be used to recover the desired derivative in substantially homogeneous form. The molecular weight of the polymer can range up to about 500,000 Daltons (D) and can be at least about 20,000 D, or at least about 30,000 D, or at least about 40,000 D. The molecular weight chosen can depend upon the effective size of the conjugate to be achieved, the nature (e.g., structure such as linear or branched) of the polymer and the degree of derivitization, i.e., the number of polymer molecules per antibody fragment, and the polymer attachment site or sites on the antibody fragment. The polymer can be covalently linked to the antibody or fragment thereof through a multifunctional crosslinking agent, which reacts with the polymer and one or more amino acid residues of the antibody or fragment to be linked. The polymer may be crosslinked directly by reacting a derivatized polymer with the antibody or antibody fragment, or vice versa. The covalent crosslinking site on the antibody or antibody fragment includes the N-terminal amino group and epsilon amino groups found on lysine residues, as well other amino, imino, carboxyl, sulfhydryl, hydroxyl, or other hydrophilic groups. The polymer may be covalently bonded directly to the antibody or antibody fragment without the use of a multifunctional (ordinarily bifunctional) crosslinking agent, as described, for example, in U.S. Pat. No. 6,458,355. The degree of substitution with such a polymer will vary depending upon the number of reactive sites on the antibody or fragment thereof, the molecular weight, hydrophilicity and other characteristics of the polymer, and the particular antibody or antibody fragment derivitization sites chosen. In general, the conjugate contains from 1 to about 10 polymer molecules, but greater numbers of polymer molecules attached to the antibodies or antibody fragments are also contemplated. The desired amount of derivitization is easily achieved by using an experimental matrix in which the time, temperature and other reaction conditions are varied to change the degree of substitution, after which the level of polymer substitution of the conjugates is determined by size exclusion chromatography or other means known and practiced in the art. Functionalized polyethylene glycol (PEG) polymers to modify the antibody or antibody fragments are available from Shearwater Polymers, Inc. (Huntsville, Ala.). Such commercially available PEG derivatives include, but are not limited to, amino-PEG, PEG amino acid esters, PEG-hydrazide, PEG-thiol, PEG-succinate, carboxymethylated PEG, PEG-propionic acid, PEG amino acids, PEG succinimidyl succinate, PEG succinimidyl propionate, succinimidyl ester of carboxymethylated PEG, succinimidyl carbonate of PEG, succinimidyl esters of amino acid PEGs, PEG-oxycarbonylimidazole, PEG-nitrophenyl carbonate, PEG tresylate, PEG-glycidyl ether, PEG-aldehyde, PEG-vinylsulfone, PEG-maleimide, PEG-orthopyridyl-disulfide, heterofunctional PEGs, PEG vinyl derivatives, PEG silanes and PEG phospholides. The reaction conditions for coupling these PEG derivatives will vary depending on the protein, the desired degree of PEGylation and the PEG derivative utilized. Some factors involved in the choice of PEG derivatives include: the desired point of attachment (such as lysine or cysteine R-groups), hydrolytic stability and reactivity of the derivatives, stability, toxicity and antigenicity of the linkage, suitability for analysis, etc. Specific instructions for the use of any particular derivative are available from the manufacturer. The resulting conjugates are separated from the unreacted starting materials by gel filtration or ion exchange HPLC.

“Monoclonal antibodies,” also designated a mAbs, are antibody molecules whose primary sequences are essentially identical and which exhibit the same antigenic specificity. Monoclonal antibodies may be produced by hybridoma, recombinant, transgenic or other techniques known to those skilled in the art.

A “non-antibody antagonist of a CCR5 receptor” (or “R5”) refers to an agent that does not comprise an antibody, and which binds to a CCR5 receptor and inhibits the activity of this receptor. Such inhibition can include inhibiting the binding of HIV-1 to the CCR5 receptor. By way of example, non-antibody antagonists include nucleic acids, carbohydrates, lipids, oligopeptides, non-chemokines and non-protein, small organic molecules.

A “small-molecule” CCR5 receptor antagonist includes, for example, a small organic molecule, or a non-protein small organic molecule, which binds to a CCR5 receptor and inhibits the activity of the receptor. Such inhibition includes, e.g., inhibiting the binding of HIV-1 to the receptor or inhibiting the entry of HIV-1 into a susceptible cell. In one embodiment, the small organic molecule has a molecular weight less than 1,500 daltons. In another embodiment, the molecule has a molecular weight less than 600 daltons.

“Subject” includes any animal or artificially modified animal capable of becoming infected with HIV. Animals include, but are not limited to, humans, non-human primates, dogs, cats, rabbits, ferrets, and rodents such as mice, rats and guinea pigs. Artificially modified animals include, but are not limited to, SCID mice with human immune systems. In an embodiment, the subject is a human. In an embodiment, the subject is a human patient.

“Synergy” between two or more agents refers to the combined effect of the agents which is greater than their additive effects. Illustratively, agents may be peptides, proteins, such as antibodies, small molecules, organic compounds, and drug forms thereof. Synergistic, additive or antagonistic effects between agents may be quantified by analysis of the dose-response curves using the Combination Index (CI) method. A CI value greater than 1 indicates antagonism; a CI value equal to 1 indicates an additive effect; and a CI value less than 1 indicates a synergistic effect. In one embodiment, the CI value of a synergistic interaction is less than 0.9. In another embodiment, the CI value is less than 0.8. In a preferred embodiment, the CI value is less than 0.7.

This invention provides a method of inhibiting HIV-2 infection of a susceptible cell by HIV-2 which comprises subjecting the susceptible cell to an effective HIV-2 infection inhibiting dose of (a) a humanized antibody designated PRO 140, or of (b) an anti-CCR5 receptor monoclonal antibody which (i) binds to CD4+CCR5+ cells and inhibits fusion of HIV-1 with such cells, (ii) inhibits HIV-1 fusion with CD4+CCR5+ cells with a potency equal or greater than that of PRO 140, (iii) coats CD4+CCR5+ cells in the subject without reducing the number of such cells in the subject, and/or (iv) binds to the subject's CD4+CCR5+ cells without inducing an increase in the subject's plasma concentration of circulating β-chemokines, wherein PRO 140 comprises (i) two light chains, each light chain comprising the light chain variable (V_{L}) and constant (C_{L}) regions encoded by the plasmid designated pVK:HuPRO140-VK (ATCC Deposit Designation PTA-4097), and (ii) two heavy chains, each heavy chain comprising the heavy chain variable (V_{H}) and constant (C_{H}) regions encoded either by the plasmid designated pVg4:HuPRO140 HG2-VH (ATCC Deposit Designation PTA-4098) or by the plasmid designated pVg4:HuPRO140 (mut B+D+I)-VH (ATCC Deposit Designation PTA-4099), wherein the effective HIV-2 infection inhibiting dose comprises from 0.1 mg per kg to 25 mg per kg of the subject's body weight, so as to thereby inhibit the infection of the susceptible cell by HIV-2. The susceptible cell may be present in a human subject. The anti-CCR5 receptor monoclonal antibody may bind to the same CCR5 epitope as that to which PRO 140 binds. The anti-CCR5 receptor monoclonal antibody may be a humanized, a human, or a chimeric antibody. The susceptible cell may be subject to an effective HIV-2 infection inhibiting dose of the antibody designated PRO 140. The antibody designated PRO 140 comprises (i) two light chains, each light chain comprising the light chain variable (V_{L}) and constant (C_{L}) regions encoded by the plasmid designated pVK:HuPRO140-VK (ATCC Deposit Designation PTA-4097) and (ii) two heavy chains, each heavy chain comprising the heavy chain variable (V_{H}) and constant (C_{H}) regions encoded by the plasmid designated pVg4:HuPRO140 HG2-VH (ATCC Deposit Designation PTA-4098). In the instant methods, HIV-2 may be of a subtype selected from subtypes A, B, C, D, E, F, G, H, J, O, or a combination thereof.

This invention also provides a method of inhibiting HIV-2 infection in an HIV-2-infected human subject which comprises administering to the subject an effective HIV-2 infection inhibiting dose of (a) a humanized antibody designated PRO 140, or of (b) an anti-CCR5 receptor monoclonal antibody which (i) binds to CD4+CCR5+ cells in the subject and inhibits fusion of HIV-1 with such cells, (ii) inhibits HIV-1 fusion with CD4+CCR5+ cells with a potency equal or greater than that of PRO 140, (iii) coats CD4+CCR5+ cells in the subject without reducing the number of such cells in the subject, and/or (iv) binds to the subject's CD4+CCR5+ cells without inducing an increase in the subject's plasma concentration of circulating β-chemokines, wherein PRO 140 comprises (i) two light chains, each light chain comprising the light chain variable (V_{L}) and constant (C_{L}) regions encoded by the plasmid designated pVK:HuPRO140-VK (ATCC Deposit Designation PTA4097), and (ii) two heavy chains, each heavy chain comprising the heavy chain variable (V_{H}) and constant (C_{H}) regions encoded either by the plasmid designated pVg4:HuPRO140 HG2-VH (ATCC Deposit Designation PTA-4098) or by the plasmid designated pVg4:HuPRO140 (mut B+D+I)-VH (ATCC Deposit Designation PTA-4099), wherein the effective HIV-2 infection inhibiting dose comprises from 0.1 mg per kg to 25 mg per kg of the subject's body weight, so as to thereby inhibit HIV-2 infection in the subject. The susceptible cell may be present in a human subject. The anti-CCR5 receptor monoclonal antibody may bind to the same CCR5 epitope as that to which PRO 140 binds. The anti-CCR5 receptor monoclonal antibody may be a humanized, a human, or a chimeric antibody. The susceptible cell may be subject to an effective HIV-2 infection inhibiting dose of the antibody designated PRO 140. The antibody designated PRO 140 comprises (i) two light chains, each light chain comprising the light chain variable (V_{L}) and constant (C_{L}) regions encoded by the plasmid designated pVK:HuPRO140-VK (ATCC Deposit Designation PTA-4097) and (ii) two heavy chains, each heavy chain comprising the heavy chain variable (V_{H}) and constant (C_{H}) regions encoded by the plasmid designated pVg4:HuPRO140 HG2-VH (ATCC Deposit Designation PTA-4098). In the instant methods, the effective HIV-2 infection inhibiting dose may be from 0.1 mg per kg to 50 mg per kg, 0.25 mg per kg to 20 mg per kg of the subject's body weight, from 0.5 mg per kg to 10 mg per kg of the subject's body weight, or from 1 mg per kg to 5 mg per kg of the subject's body weight. The effective HIV-2 infection inhibiting dose may be 5 mg per kg of the subject's body weight, 10 mg/kg of the subject's body weight, or 20 mg/kg of the subject's body weight.

The effective HIV-2 infection inhibiting dose may be administered at regular intervals. The effective HIV-2 infection inhibiting dose may be sufficient to achieve in the subject a serum concentration of the antibody of at least 400 ng/ml. The effective HIV-2 infection inhibiting dose may be sufficient to achieve and maintain in the subject a serum concentration of the antibody of at least 1 μg/ml. The effective HIV-2 infection inhibiting dose may be sufficient to achieve and maintain in the subject a serum concentration of the antibody of about 3 to about 12 μg/ml. The effective HIV-2 infection inhibiting dose may be sufficient to achieve and maintain in the subject a serum concentration of the antibody of at least 5 μg/ml. The effective HIV-2 infection inhibiting dose may be sufficient to achieve and maintain in the subject a serum concentration of the antibody of at least 10 μg/ml or at least 15 ug/ml, or at least 20 ug/ml. The effective HIV-2 infection inhibiting dose may be sufficient to achieve and maintain in the subject a serum concentration of the antibody of at least 25 μg/ml, or at least 30 ug/ml, or at least 35 ug/ml. The effective HIV-2 infection inhibiting dose may be sufficient to achieve and maintain in the subject a serum concentration of the antibody of at least 50 μg/ml. The effective HIV-2 infection inhibiting dose may be administered at one or more predefined intervals. The predefined interval may be at least once weekly, every two to four weeks, every two weeks, every three weeks, is every four weeks, at least once monthly, every six weeks, or every eight weeks. In the instant methods, the resistant HIV-2 virus may be of a subtype selected from subtypes A, B, C, D, E, F, G, H, J, O, or a combination thereof. The antibody may be administered via intravenous infusion or via subcutaneous injection.

The instant methods of the invention may further comprise administering to the subject at least one additional antiretroviral agent effective against HIV-2. The antiretroviral agent may be a CCR5 antagonist that does not compete with the humanized antibody designated PRO 140 of (a), or the anti-CCR5 receptor monoclonal antibody of (b). The CCR5 antagonist may be an antibody. The antibody may be a monoclonal antibody, a humanized antibody, a human antibody, or a chimeric antibody. The CCR5 antagonist may be a non-antibody, small-molecule CCR5 antagonist. The subject may be treatment-naïve or treatment-experienced.

This invention also provides a method of inhibiting in a human subject the onset or progression of an HIV-2-associated disorder, the inhibition of which is effected by inhibiting fusion of HIV-2 which comprises administering to the subject at a predefined interval effective fusion-inhibitory doses of a humanized antibody designated PRO 140, or of an anti-CCR5 receptor antibody which (i) binds to CD4+CCR5+ cells in the subject and inhibits fusion of HIV-1 with such cells, (ii) inhibits HIV-1 fusion with the subject's CD4+CCR5+ cells with a potency characterized by an IC90 of 10 μg/ml or less, (iii) coats the subject's CD4+CCR5+ cells without reducing the number of such cells in the subject, and/or (iv) binds to the subject's CD4+CCR5+ cells without inducing an increase in the subject's plasma concentration of circulating β-chemokines, wherein PRO 140 comprises (i) two light chains, each light chain comprising the light chain variable (V_{L}) and constant (C_{L}) regions encoded by the plasmid designated pVK:HuPRO140-VK (ATCC Deposit Designation PTA-4097), and (ii) two heavy chains, each heavy chain comprising the heavy chain variable (V_{H}) and constant (C_{H}) regions encoded either by the plasmid designated pVg4:HuPRO140 HG2-VH (ATCC Deposit Designation PITA-4098) or by the plasmid designated pVg4:HuPRO140 (mut B+D+I)-VH (ATCC Deposit Designation PTA-4099), wherein each administration of the antibody delivers to the subject from 0.1 mg per kg to 25 mg per kg of the subject's body weight, so as to thereby inhibit the onset or progression of the HIV-2-associated disorder in the subject.

This invention further provides a method of reducing the likelihood of a human subject's contracting infection by HIV-2 which comprises administering to the subject at a predefined interval effective fusion-inhibitory doses of a humanized antibody designated PRO 140, or of an anti-CCR5 receptor antibody which (i) binds to CD4+CCR5+ cells in the subject and inhibits fusion of HIV-1 with such cells, (ii) inhibits HIV-1 fusion with the subject's CD4+CCR5+ cells with a potency characterized by an IC90 of 10 μg/ml or less, (iii) coats the subject's CD4+CCR5+ cells without reducing the number of such cells in the subject, and/or (iv) binds to the subject's CD4+CCR5+ cells without inducing an increase in the subject's plasma concentration of circulating β-chemokines, wherein PRO 140 comprises (i) two light chains, each light chain comprising the light chain variable (V_{L}) and constant (C_{L}) regions encoded by the plasmid designated pVK:HuPRO140-VK (ATCC Deposit Designation PTA-4097), and (ii) two heavy chains, each heavy chain comprising the heavy chain variable (V_{H}) and constant (C_{H}) regions encoded either by the plasmid designated pVg4:HuPRO140 HG2-VH (ATCC Deposit Designation PTA-4098) or by the plasmid designated pVg4:HuPRO140 (mut B+D+I)-VH (ATCC Deposit Designation PTA-4099), wherein each administration of the antibody delivers to the subject from 0.1 mg per kg to 25 mg per kg of the subject's body weight, so as to thereby reduce the likelihood of the subject's contracting an HIV-2 infection. The subject may have been exposed to HIV-2, or may be at risk of being exposed to HIV-2.

The subject may be treatment-naïve, i.e., the subject has not previously undergone treatment with any anti-HIV-2, antiretroviral agents. The subject may also be treatment-experienced, i.e., the subject has undergone, and/or is undergoing, treatment with one or more anti-HIV-2, antiretroviral agents. Diagnostic assessment of subjects undergoing treatment with PRO 140, alone or in combination with other antiretrovirals, including other CCR5 receptor antagonists, are encompassed by this invention. In an embodiment, a subject to be treated with PRO 140 is tested prior to treatment to assess the subject's HIV tropism. Tropism refers to the affinity of a virus for a specific co-receptor on a target cell. The subject may be treatment-experienced or treatment-naive. Viral tropism may be assessed or screened by procedures known in the art, such as the Trofile™ Assay (Monogram Biosciences, South San Francisco, Calif.), which can provide an HIV profile for a subject, i.e., the strain of virus (R5, X4, or D/M (dual/mixed (R5/X4)) that infects the subject. A subject determined to be infected with HIV-2 may then undergo treatment with PRO 140. An embodiment of the invention is therefore directed to a method of treating an HIV-1-infected subject with PRO 140 to reduce viral load in the subject, wherein the subject is diagnostically determined to be infected with CCR5-tropic HIV-2 prior to treatment, and then is treated with PRO 140 in accordance with any of the treatment and dosing methods described herein. In an embodiment, a subject is screened for CCR5 viral tropism about one to six weeks before treatment with PRO 140. In an embodiment, a subject is screened for CCR5 viral tropism about three to six weeks before treatment with PRO 140. In an embodiment, a subject is screened for CCR5 viral tropism about two to five weeks before treatment with PRO 140. In an embodiment, a subject is screened for CCR5 viral tropism about a month to a month and a half before treatment with PRO 140.

In another embodiment, a subject is monitored and screened at repeated intervals during the course of treatment with PRO 140 to determine HIV tropism according to procedures known and used by those skilled in the art. In this way, a subject's drug regimen (e.g., dosing and/or co-administration of other antiretrovirals with PRO 140) can be adjusted or modified as necessary or required according to the subject's virus tropism profile over time. In an embodiment, the subject is determined to be infected with HIV-2 prior to treatment with PRO 140, with or without other antiretrovirals. Illustratively, monitoring of viral tropism in a subject who is being treated with PRO 140, alone or in combination with other antiretrovirals, may be maintained for a period of six months, one year, two years, three years, four years, five years or longer, as necessary, after treatment is begun. In an embodiment, monitoring a subject for a change in viral tropism may be correlated with other parameters, such as CD4 cell count and viral load. For example, a change in treatment may not be warranted if a change in tropism in a subject undergoing treatment occurs in the absence of any effects on viral load or CD4 cell count in that subject. In addition, a relative increase in X4 virus versus an absolute increase in X4 virus in a patient being treated can be assessed to determine optimization or assessment of a subject's HIV treatment regimen. A relative increase in X4 tropic virus may reflect an increased chance of detection, and may not be as significant if observed during monitoring as an absolute increase in X4 tropic virus, since an absolute increase in X4 tropic virus may reflect a potentially preferential expansion of the X4 virus population in the subject. One skilled in the art will further appreciate that if a subject's CCR5-tropic HIV infection is being inhibited and viral load is being reduced by the use of a CCR5 receptor antagonist, an increase in the detection of X4 virus, if present, might be expected, even in the absence of any absolute increase in the amount of X4 virus, as a result, for example, of depletion of CCR5-tropic HIV. (Report of an FDA/FCHR Joint Public Meeting, May 31, 2006, Forum for Collaborative HIV Research, Apr. 24, 2007). Thus, virus tropism monitoring should be conducted with such outcomes in mind.

In an embodiment, a subject undergoing treatment with PRO 140, alone or in combination with other antiretroviral drugs, is tested for HIV drug resistance at predetermined intervals during the course of treatment. A non-limiting example of a widely used phenotypic HIV drug resistance test is the PhenoSense™ HIV assay, which measures the sensitivity of a virus to antiretroviral drugs. For example, it has been found that the in vitro susceptibility data obtained in the PhenoSense™ HIV Entry Assay is in good agreement with data obtained from testing the same patient-derived viral envelopes in PBL. Thus, this assay, or similar assays, may be used as a primary screen for testing patient samples for resistance to an antiretroviral CCR5 entry inhibitor. A clinician or practitioner is able to determine the level of susceptibility that a person has to each antiretroviral drug in order to design an individualized treatment regimen. In addition, such resistance testing and assessment may be continued in a subject receiving PRO 140 as a treatment regimen, alone or in combination with other antiretroviral drugs, to provide follow-up of the treated subjects at predetermined intervals.

In an embodiment, subjects who are undergoing treatment with PRO 140, alone, or in combination with other antiretroviral drugs, which may include other CCR5 receptor antagonists, are monitored for the development of tumors, e.g., lymphomas and sarcomas, and malignancies at repeated intervals. Without limitation, such intervals may be established to be, for example, once a month, twice a month, once every three weeks, once every six weeks, once every two to six months, or two to six times a year. In an embodiment, subjects who are undergoing treatment with PRO 140, alone or in combination with other antiretroviral drugs which may include other CCR5 receptor antagonists, are monitored for the development of infections (bacterial, viral, opportunistic, etc.). Monitoring of subjects receiving treatment with one or more CCR5 receptor antagonists may include assessment, at the same or at different times, of, for example, virus tropism changes, viral resistance, viral load (HIV RNA levels), CD4 cell count and tumor/malignancies, etc., at repeated intervals during the treatment, e.g., on a monthly basis, every six weeks, every eight weeks, every ten weeks, every twelve weeks, or 2-3 times per year. Such assessments further involve the storage of baseline samples, e.g., serum, taken from the subject prior to and/or at the time of beginning a treatment regimen. Additionally, molecular clonal analysis of the virus population(s) in a subject at baseline may be assessed using methods known and practiced in the art. In this way it can be determined that any tropism change in a subject's virus population (e.g., a CXCR4 variant or dual/mixed virus) emerged from a pre-existing reservoir in the subject not detected at baseline and not from a co-receptor use change in the subject. For each of the above embodiments directed to follow-up, monitoring and periodic screening of subjects undergoing treatment for HIV infection, those skilled in the art will be able to determine the appropriate time intervals in which such follow-up, monitoring and screening assessments should be made.

In accordance with the various methods of the present invention, it will be appreciated that the humanized anti-CCR5 monoclonal antibody PRO 140 possesses a distinct pattern of viral resistance, synergizes with small molecule drugs, blocks HIV without CCR5 antagonism in vitro, exhibits a potential for improved tolerability, enables infrequent dosing and is not expected to be involved in drug-drug or food interactions and is well tolerated in human subjects based on preclinical studies as described hereinbelow. Thus, PRO 140 is advantageously used alone or in combination with other antiretroviral drugs or agents in methods of treating HIV infection and in methods of reducing viral load in an HIV infected patient.

### Short Term, Interim, or Induction Use:

Either upon initiation of first HIV therapy regimen, or upon switch of therapy (first line to second line, etc.) the objective of antiviral therapy is to maximally suppress viral load as quickly as possible. Use of PRO-140 in combination with other antiretroviral drugs, even for a short period of time (+/−3 months), can help to ensure rapid and full viral suppression to <50 copies (HIV RNA/ml3). Use of PRO-140 could be continued for a minimum of 12 weeks or until full viral suppression (<50 copies) is achieved. Whether dosed once monthly IV or once weekly subcutaneously, PRO-140 used in an induction format can assist in rapidly suppressing viral replication, protecting the susceptibility of concurrent HIV drugs, as well as sensitivity of patient virus to subsequent HIV drugs. Use of PRO-140 in this manner coincides with the current standard of care at the start of HIV therapy, or upon treatment switching, where frequent viral load testing is conducted (up to 1×/week in the first month, and or 1×/month in the first three months), facilitating PRO-140 administration (e.g., monthly) at the time of clinic visits for laboratory testing blood draws. The concept of induction/maintenance is much like the model often used in cancer of ablation upfront, followed by maintenance (lower/less intensive) chemotherapy for a period thereafter. PRO-140 is used for a short period of time, say 3-6 months, in combination with other anti-retrovirals (or alone) in order to rapidly and completely suppress HIV viral replication and stimulate CD4+ cell proliferation. Once desired levels are achieved and confirmed through repeated lab tests (2 viral load tests indicating <50 copies/ml3 and/or >100 CD4+ cell increase), PRO-140 use could be stopped, while patients continue with other anti-retroviral agents to maintain these levels of suppression and CD4+ immune system status.

PRO-140 “intensifies” the potency/effectiveness of an antiretroviral regimen, for patients who are either new (naive) to therapy or those who are switching therapy due to inadequate virologic or immunologic response to prior therapy. PRO-140 would be used in an acute and temporary manner with this approach to achieve a desired result and then cease using it, rather than using it chronically even after an endpoint is met as with most anti-HIV drugs today. Being able to dose PRO-140 once every month helps to render this approach more feasible as it coincides with normal blood draws following HIV therapy initiation or switch.

PRO 140 can be administered to HIV-2 infected patients who are transitioning from one drug regimen to another. PRO 140 can be administered to the patient during the interim time period between one drug regimen and a second drug regimen of different drugs, or different drug combinations, and/or different drug doses, etc. PRO-140 can safely be removed from the combination of anti-HIV drugs used to achieve full suppression, once viral load has reached <50 copies following two separate lab tests. This is the case where at least two, but preferably three, active drugs are used in the follow-on (maintenance or subsequent) regimen.

### Intermittent Viral Load Detection

Temporary use of PRO-140 is also appropriate in cases where patients have viral load that is generally suppressed to <50 or <400 copies, but occasionally rises to levels exceeding these thresholds. Use of PRO-140 for one to three months following two viral load tests confirming ‘viral escape’ may support the patients current HIV therapy and effectively re-suppress viral replication. Use of PRO-140 even in this short term modality may also afford important immune system restoration function in the form of CD4+ proliferation to further improve patients clinical status.

Persistent, Low level Viral Replication

It is common among treatment experienced patients to see incomplete viral suppression, or stable, low-level viral replication (>400 but <10,000 copies). In such cases, clinicians often allow patients to continue their HIV regimen as long as there is no change in clinical status or CD4+ count. Temporary (+/−3 month) use of PRO-140 may assist clinicians in suppressing viral loads to <50 copies, even in patients who have never reached this objective, with or without changing some/all of the patients other concurrent anti-HIV medications. Use of PRO-140 even in this short term modality may also afford important immune system restoration function in the form of CD4+ proliferation to further improve patients clinical status.

Boosting CD4 Count in Patients with CCR5, Dual Mixed or CXCR4 Tropic Virus

In treatment experienced patients or patients infected with multi-drug resistant HIV virus, often the primary goal of therapy is not to suppress HIV viral load but to sustain or improve immune system (CD4+ cell) function. Use of PRO-140 in such patients, either alone or in combination with other antiretroviral agents and regardless of HIV virus tropism, may help boost CD4+ cell count and stabilize a patient's clinical status thus reducing the risk of HIV disease progression.

### Re-Use or Recycling of PRO-140

Other entry inhibitors, specifically enfuvirtide, have published data demonstrating that the drug may have residual activity in up to 50% of patients who have documented resistance and treatment failure to that drug. Following discontinuation of enfuvirtide therapy for 60-90 days, genetic mutations in the gp41 envelope region (36-45) appear to revert to wild-type status. Upon re-initiation of enfuvirtide therapy, up to 50% of patients achieve a response in viral load reduction of ˜1 log which is sustained for at least 6 months. Among patients who reinitiated enfuvirtide therapy and did not respond, resistance mutations in the gp41 envelope region differed from those seen in prior enfuvirtide therapy. PRO-140 possesses the same characteristics in inducing conformational changes in the V3 loop region, that differ upon reintroduction of drug following prior documented resistance and treatment failure. This renders recycling or reuse of PRO-140 a viable therapeutic approach.

### Extra-Cellular Only HIV Regimens

With the development of numerous anti-HIV compounds whose mechanism of action focuses on prevention of HIV virus entry into the target (CD4+) immune system cells, it may be possible to fully suppress HIV replication by using combinations of such extra-cellularly active drugs alone. This implies the potential for simplified HIV regimens (fewer drugs needed) that block viral entry and “protect” immune system cells. This approach also has the potential to reduce drug interactions, drug related toxicities as well as exacerbation of co-morbidities often seen in HIV patients (hepatitis, etc.). Use of PRO-140 with other extracellularly active anti-HIV drugs that target either HIV or host proteins (including gp41 fusion inhibitors, CCR5, CXCR4, gp120 or other moieties) could be sufficient to fully suppress HIV replication in a sustained manner. This would avoid the need for co-administration of NRTIs, nNRTI's, protease inhibitors (PIs) or integrase inhibitors with PRO-140.

Leveraging Synergistic Mechanistic Activity with PIs

Evidence exists to support the selective use of HIV viral entry inhibitors with ritonavir boosted protease inhibitors to achieve synergistic MOA based activity that results in enhanced viral suppression compared to combinations of anti-HIV drugs from other classes. By preventing HIV viral entry in to CD4+ cells, as well as preventing HIV viral expression from CD4+ cells after intracellular incorporation, PRO-140 and protease inhibitors may induce greater, and/or more rapid, and/or more complete HIV viral suppression than the combination of other mechanisms of action. This synergy could provide the rationale to preferentially use PRO-140 with protease inhibitors, with or without other anti-HIV drugs, to achieve maximal viral suppression and CD4+ proliferation.

Co-Formulation with Other Anti-HIV Drugs

Based on the above information, as well as the established precedent in HIV therapy to date, ample rational exists for the co-formulation of PRO-140 with other anti-HIV drugs to enable combined administration. Such co-formulation could involve other injectable anti-HIV drugs or oral anti-HIV drugs that are reformulated into parenteral forms.

### PRO-1140 Use to Impair Viral Fitness and Pathogenicity

Use of PRO-140 in either lower (than therapeutically necessary) doses or less frequently (dosed) in order to exert sufficient pressure that forces the HIV virus to mutate and reduces the efficacy of PRO-140; however, as a result, a virus that is less virulent, pathogenic or ‘fit’ (less capable to replicate) is produced. This might be an application suited for patients whose virus has developed resistance to PRO-140, but who are still deriving some type of immunologic benefit (sustained or rising levels of CD4+ cells—also termed discordant response) and thus may still derive benefit from continuing PRO-140 therapeutic treatment. Such a debilitated HIV virus may also be more susceptible to other HIV drugs, improving their effect on HIV viral suppression or CD4+ response.

### PRO-140 Use in Immune Cell Mobilization

Given the early and robust proliferation in CD4+ cells from the Phase 1b study, it is possible that PRO-140 exhibits a mechanism of action that potently effect both active and resting CD4+ cells, as well as other immune system cells, in a manner that is different from other entry inhibitors. Use of PRO-140 in single or multiple doses to stimulate or accelerate immune system cell proliferation may be appropriate and justified, whether in HIV infected patients, whether naive to HIV therapy, currently on therapy or who have ceased HIV therapy due to resistance or other reasons.

The following Experimental Details are set forth to aid in an understanding of the subject matter of this disclosure, but are not intended to, and should not be construed to, limit in any way the claims which follow thereafter.

### EXPERIMENTAL DETAILS

### Part I

### Materials and Methods

Compounds and mAbs

PRO 140 was prepared by expression in Sp2/0 cells using Hybridoma serum-free medium supplemented with 2 mM L-glutamine (Invitrogen, Carlsbad, Calif.). Bulk mAb was clarified using a 5.0 μm Depth filter (Sartorius, Goettingen, Germany) followed by passage over a 0.2 μm sterilizing grade filter (Sartorius). The mAb was purified by passage first over an affinity column (MabSelect Protein A column, Amersham, Piscataway, N.J.) and then by ion exchange chromatography (SP Sepharose Cation Exchange resin, Amersham). PRO 140 was nanofiltered using a Viresolve™ 10 Opticap NFP capsule (Millipore, Billerica, Mass.) followed by a 0.2 μm filter and concentrated/diafiltered over disposable TFF cartridges (Millipore). The mAb was then polished over a hydroxyapatite column (Bio-Rad, Hercules, Calif.), concentrated to 10 mg/ml in phosphate-buffered saline and stored at −70° C. or colder prior to use.

RANTES was purchased from R&D Systems (Minneapolis, Minn.). The anti-CCR5 mAb 2D7 was purchased from BD Biosciences (Cat. #555993), and the anti-CCR5 mAb CTC5 was purchased from R&D Systems (Cat. #FAB1802P).

### RET Assay

The HIV-1 RET assay has been described in detail previously (Litwin et al., 1996). Briefly, fluorescein octadecyl ester (F18; Molecular Probes, Eugene, Oreg.; 5 mg/ml in ethanol), was diluted 1:800 in DMEM labeling medium (DMEM; Invitrogen, Carlsbad, Calif.) with 10% fetal bovine serum (FBS; HyClone, Logan, Utah) and adjusted to an A₅₀₆ of 0.34±10%. Octadecyl rhodamine B chloride (R18; Molecular Probes; 10 mg/ml in ethanol) was diluted 1:2050 in labeling medium and adjusted to an A₅₆₅ of 0.52±10%. Both dyes were further diluted 2-fold by addition to cells in T75-cm² flasks. HeLa-Env_{JRFL} and CEM NKR-CCR5 cells were incubated overnight in F18- and R18-containing culture medium, respectively. The following day, medium from HeLa-Env_{JRFL} cells was removed and 10 ml of 0.5 mM EDTA was added and incubated at 37° C. for 5 min. EDTA was removed and the flask was returned to the incubator for another 5 min followed by striking of the flask to dislodge cells. Ten ml of PBS- with 15% FBS were added to the flask and the contents were transferred to a 50-ml conical centrifuge tube. Suspension CEM NKR-CCR5 cells were added directly to a separate 50-ml conical centrifuge tube. Both cell lines were centrifuged at 300×g for 5 min. The supernatant was discarded and cells were resuspended in 10 ml of PBS-/15% FBS. The centrifugation/wash step was repeated twice, after which the cells were counted and concentrations adjusted to 1.5×10⁶ cells/ml. Ten μl of each cell type (15,000 cells) were seeded into wells of a 384-well plate. Inhibitor compounds were added immediately thereafter to bring the final well volume to 40 μl, and the plates were incubated for 4 h at 37° C. Compounds were tested individually and in combination at a fixed molar ratio or mass ratio over a range of serial dilutions. The plates were then read on a fluorescence plate reader (Victor², Perkin Elmer, Boston, Mass.) using the excitation/emission filter combinations shown in Table 6.

TABLE 6
Excitation/emission filter combinations for RET assay

| Scan No. | Excitation wavelength | Emission wavelength |
|---|---|---|
| 1 | 450 nm/50 nm | 530 nm/25 nm |
| 2 | 530 nm/25 nm | 590 nm/35 nm |
| 3 | 450 nm/50 nm | 590 nm/35 nm |

The “% RET” was calculated according to the following formula after subtraction of background (blank) readings:

% RET=100×[(A₃−(A₁×Fₛₚᵢₗₗ)−(A₂×Rₛₚᵢₗₗ))/A₂]

### Where:

Fₛₚᵢₗₗ=HeLa cells alone, Scan 3/Scan 1;
Rₛₚᵢₗₗ=CEM cells alone, Scan 3/Scan 2;
A₁=Scan 1 value for HeLa and CEM cells in combination;
A₂=Scan 2 value for HeLa and CEM cells in combination; and
A₃=Scan 3 value for HeLa and CEM cells in combination.

The “% Inhibition” was calculated according to the following formula:

% Inhibition=100×[(Max % RET−% RET for sample well)/(Max % RET−Min % RET)]

### Where:

Max % RET=average of % RET values for HeLa and CEM cell combination without added inhibitor; and
Min % RET=average of % RET values for HeLa and CEM cell combination in presence of 500 ng/ml of Leu-3a mAb (an antibody that targets CD4 and fully blocks fusion in the RET assay at this concentration).

Fifty percent inhibition (IC₅₀) values were determined by fitting the inhibition data with a non-linear, four-parameter, variable slope equation (GraphPad Prism, 4.02; GraphPad Software, San Diego, Calif.). Upper and lower inhibition values were constrained to 100% and 0%, respectively for curve fitting.

### Preparation of PBMCs

Replication of authentic HIV-1 is measured in activated peripheral blood mononuclear cells (PBMCs) using the monocyte/macrophage-tropic HIV-1 clone, JRFL (HIV-1_{JRFL}), for these studies.

PBMCs are isolated from 4 separate donors (Leukopacks) by centrifugation on a Ficoll gradient. CD8 cells are depleted using RosetteSep CD8 Depletion Cocktail (#15663, StemCell Research, Vancouver, BC). Cells are diluted to 4×10⁶/ml and added in equal parts to three T175-cm² flasks and then stimulated by addition of one of the following media: IL-2 Medium [RPMI 1640 (#10-040-CV, Cellgro, Herndon, Va.), 10% FBS (#35-010-CV), 2 mM L-Glutamine (#25-005-CI), 100 U/ml IL-2 (Sigma, St. Louis, Mo.)]; PHA 5 Medium: [IL-2 Medium with 5 ug/ml Phytohemagglutinin PHA-P (PHA) (#L8754, Sigma, St. Louis, Mo.), filtered]; or PHA 0.5 Medium: [IL-2 Medium with 0.5 ug/ml PHA, filtered]. Each flask receives a total of 50-150 ml of medium. Flasks are incubated for 3 days at 37° C. followed by pooling of the contents prior to use in the infection assay.

### Virus Titration

Serial dilutions of virus are tested in quadruplicate on activated PBMCs (1.4×10⁵ PBMC/well). Titration Medium [IL-2 Medium with 100 IU/ml penicillin/streptomycin (#30-002-CI, Cellgro)] is utilized for virus titrations. Fifty μl of diluted virus is added to 100 μl of PBMCs in flat bottom, tissue-culture treated 96-well plates (VWR# 29442-054, Corning, Corning, N.Y.) and the plates are incubated at 37° C. in a humidified, 5% CO₂ incubator. After 7 days, 50 μl are removed from each well and tested for virus levels by p24 antigen ELISA (Perkin Elmer, Boston, Mass.). Virus titer is determined by the method of Reed and Muench (Table 11, see below).

### Neutralization Assay

Stimulated PBMCs are seeded into wells of 96-well flat bottom plates at a density of 1.4×10⁵ cells/well. Virus is diluted to 2,000 TCID₅₀/ml and mixed with serial 0.5 log₁₀ dilutions of compound for 1 h at 37° C. prior to addition to the cell plates. The final amount of virus added per well is 100 TCID₅₀. The final DMSO concentration in the assay is always 0.5% whenever small molecule inhibitors are being tested. Plates are incubated at 37° C. for 5 days, at which time an aliquot of supernatant is removed for p24 antigen ELISA. If control wells (virus without inhibitor) exhibit low p24 antigen levels then the plates are brought back to full volume with Titration medium and incubated for an additional 24 h.

### Data Analysis

Neutralization activity is displayed by plotting the percent inhibition of p24 antigen production (after background values are subtracted from all datapoints) versus log₁₀ drug concentration. The percent inhibition is derived as follows [1−(p24 levels in the presence of drug/p24 levels in the absence of drug)]×100. IC₅₀ values are determined by fitting the inhibition data with a non-linear, four-parameter, variable slope equation (GraphPad Prism, ver. 4.02; GraphPad Software, San Diego, Calif.). Upper and lower inhibition values are constrained to 100% and 0%, respectively for curve fitting.

### Phase 1a Clinical Study

Individuals were treated in sequential, dose-rising cohorts of 5 subjects (4 active and 1 placebo) each and evaluated for up to 120 days post-treatment. A population of healthy, i.e., HIV-1 uninfected, male volunteers with no abnormal findings on physical exam, medical history and ECG, aged 19-50, was administered a single intravenous infusion of PRO 140 (0.1, 0.5, 2.0 and 5.0 mg per kg body weight). Safety assessments consisted of monitoring the following: vital signs (blood pressure, pulse, temperature, etc; hematology (hemoglobin, hematocrit, leukocytes, platelets, etc.); serum chemistries (AST/ALT, alkaline phosphatase, BUN, creatinine, etc.); urinalysis (pH, specific gravity, protein, glucose, leukocytes, etc.); and ECGs (12-lead).

### Measurement of Coating of CCR5 Cells by PRO 140

Whole blood specimens were combined separately with the indicated phycoerythrin-labeled anti-CCR5 antibodies or with appropriate isotype-control antibodies. Erythocytes were lysed and leukocytes were stabilized using the ImmunoPrep Reagent System (Beckman Coulter), and the cells were analyzed on a TQ Prep™ flow cytometry workstation (Beckman Coulter). Data were expressed as the percent of CCR5 cells relative to all cells gated in the analysis. CTC5 is an anti-CCR5 antibody that does not compete with PRO 140. 2D7 is an anti-CCR5 antibody that does compete with PRO 140.

### Measurement of Serum Concentrations of PRO 140

Sera were diluted as appropriate and combined with L1.2-CCR5 cells, which are mouse pre-B lymphoma cells engineered to stably express human CCR5. In order to generate a standard curve, PRO 140 standard was tested in parallel at concentrations ranging from 0.062 to 4.0 μg/ml in 10% normal human serum (NHS). 10% NHS containing no PRO 140 was analyzed as a negative control. Following incubation with test samples, cells were washed and combined with a FITC-labeled sheep antibody against human IgG4 (The Binding Site Limited, Cat. #AF009). Cells were washed again and analyzed by flow cytometry. The concentration of PRO 140 was determined by comparing the median fluorescence intensity (MFI) of the test sample with MFI values of the standard curve.

### Determination of Plasma RANTES Concentration

The assay employed the Quantikine™ Human RANTES Immunoassay Kit (R&D Systems, Minneapolis, Minn.). Briefly, platelet-poor plasma was collected in CTAD/EDTA tubes and stored at −20° C. Test samples and RANTES standard were added to microtiter plates that were pre-coated with a mouse monoclonal antibody to RANTES. Following incubation, plates were washed and contacted with an anti-RANTES polyclonal antibody conjugated to horseradish peroxidase (HRP). Plates were washed again prior to addition of tetramethlybenzidine substrate for colorimetric detection. The Lower Limit of Quantification of the assay was 415 pg RANTES/ml plasma.

### Results and Discussion

PRO 140 is a humanized IgG4,K anti-CCR5 mAb being developed for HIV-1 therapy. This antibody has been shown to broadly and potently inhibit CCR5-mediated fusion of HIV-1 to target cells in vitro. PRO 140 is also highly active in a therapeutic hu-PBL-SCID mouse model, and preliminary data are now available from a Phase 1a clinical study in healthy human subjects.

### In Vitro Antiviral Activity of PRO 140

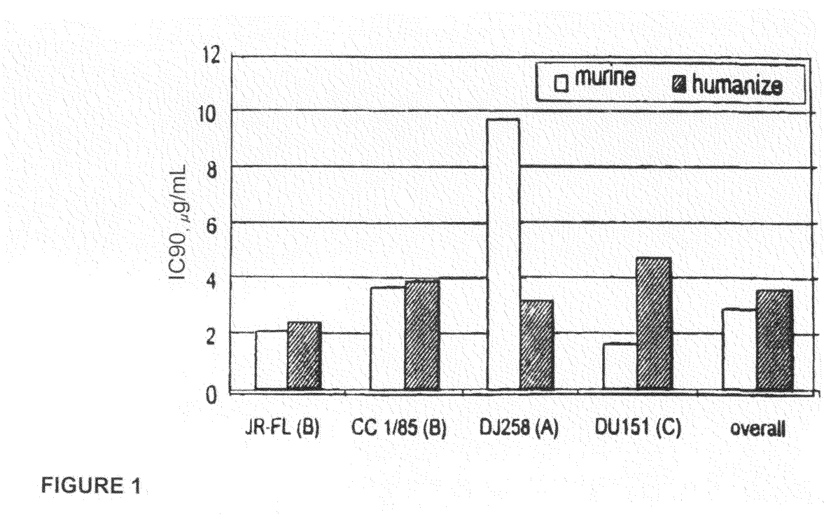

Murine and humanized PRO 140 were tested against four primary R5 HIV-1 isolates as described in the Methods. FIG. 1 shows that PRO 140 has potent antiviral activity in vitro, neutralizing a variety of primary R5 strains with an IC90 of 3-4 μg/ml. PRO 140 exhibited similar antiviral activity to the murine mAb, PA14, from which PRO 140 is derived.

Preliminary Data from Phase 1a Clinical Study

The primary objective of the Phase 1a study was to evaluate the safety and tolerability of PRO 140 given as a single dose in a rising dose cohort regimen in healthy male subjects. The secondary objectives were (1) to gain information about the pharmacokinetics of intravenously administered PRO 140, and (2) to gain information on the effects of PRO 140 on blood levels of CCR5+ cells and chemokines.

### Pharmacokinetics of PRO 140

Healthy male volunteers were treated with a single intravenous infusion of PRO 140 at dose levels of 0.1, 0.5, 2.0 and 5.0 mg/kg. PRO 140 and placebo were generally well tolerated with no significant changes in ECGs and no dose-limiting toxicity.

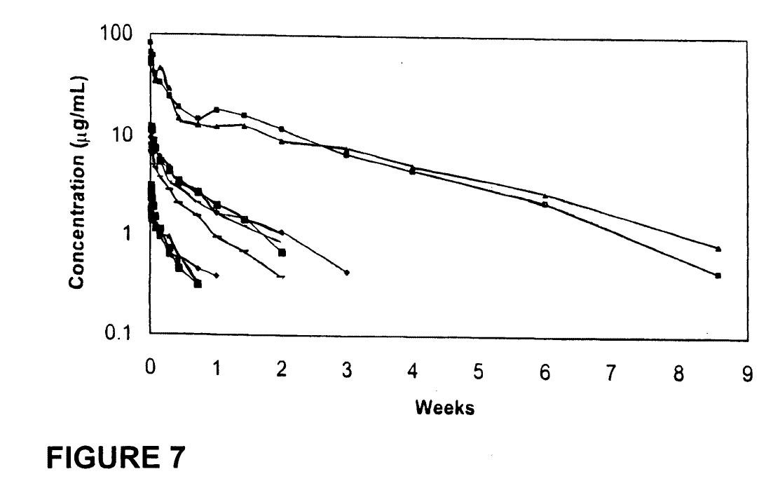

Serum was collected post-treatment, cryopreserved, and analyzed for PRO 140 levels. Peak serum concentrations ranged to 3 mg/ml at 0.1 mg/kg and 12 mg/ml at 0.5 mg/kg. Serum concentrations remained detectable (>400 ng/ml for up to 5 days at 0.1 mg/kg, 21 days at 0.5 mg/kg, and for over 60 days following a single 2 mg/kg injection (FIG. 7). Serum concentrations of PRO 140 increased proportionally with dose level, and the clearance rate was similar to that of other humanized mAbs. Pharmacokinetic (PK) metrics were determined using WinNonLin (PharSight Corporation, Mountain View, Calif.) using a noncompartmental model, and the terminal serum half-life of PRO 140 was determined to be 10-12 days. As expected, no subject developed antibodies to the humanized PRO 140.

### Coating and Non-Depletion of CCR5 Lymphocytes by PRO 140

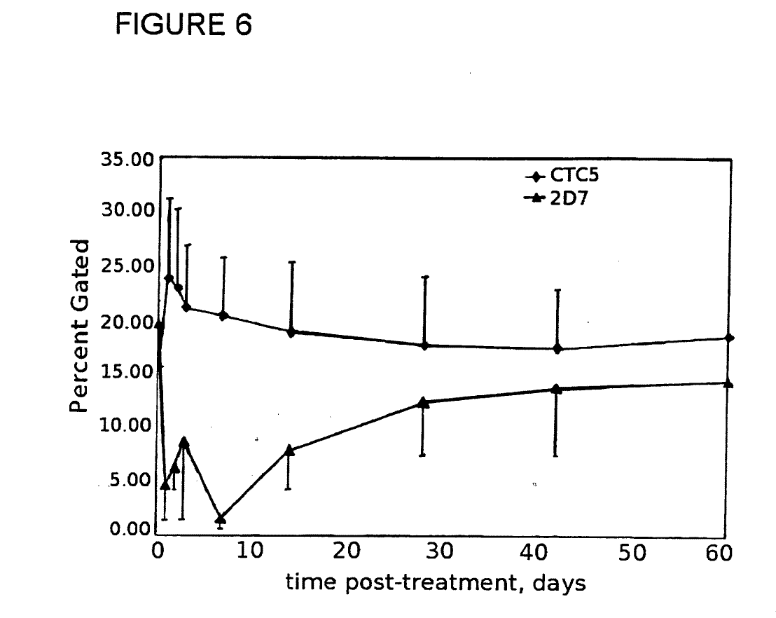

Healthy male volunteers (n=4) were treated with a single intravenous infusion of PRO 140 at a dose level of 2 mg/kg. For up to 60 days post-treatment, at the times indicated in FIG. 6, blood was collected and analyzed for CCR5 lymphocyte levels.

Following treatment with PRO 140, there was no decrease in the overall number of CCR5 lymphocytes at measured by CTC5 binding; however, the binding of antibody 2D7 was significantly decreased (FIG. 6). Background binding of isotype control antibodies was unchanged. Since the binding of CTC5 is not decreased by the presence of PRO 140, the CTC5-PE values are a measure of the total number of circulating CCR5 lymphocytes. Since 2D7 competes with PRO 140, the 2D7-PE values reflect the number of CCR5 lymphocytes that are not coated with PRO 140.

The data indicate that a single 2 mg/kg dose of PRO 140 effectively coats CCR5 lymphocytes without cellular depletion for two weeks, and cells remain partially coated for >4 weeks. In addition, CCR5 coating was more prolonged in patients treated with 5 mg/kg PRO 140. The data indicate that a single 5 mg/kg dose of PRO 140 effectively coats CCR5 lymphocytes without cellular depletion and the cells remain partially coated for >60 days (FIG. 10). Since CCR5 coating is the mechanism whereby PRO 140 inhibits HIV, viral loads in HIV-infected individuals could be expected to decrease in a similar temporal manner.

### Effect of PRO 140 on Plasma Chemokine Levels

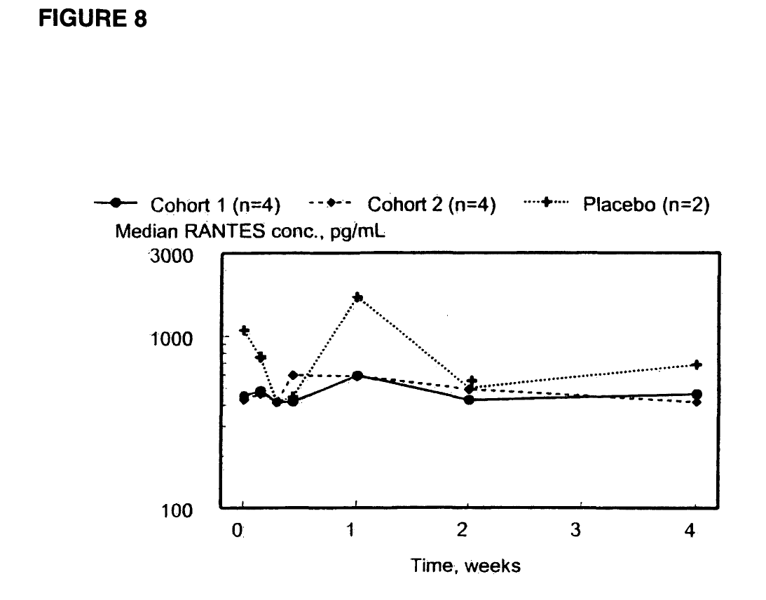

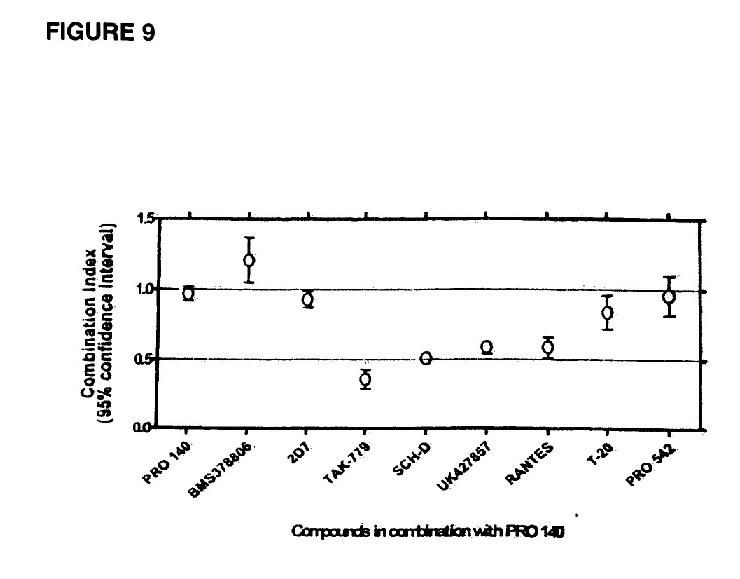

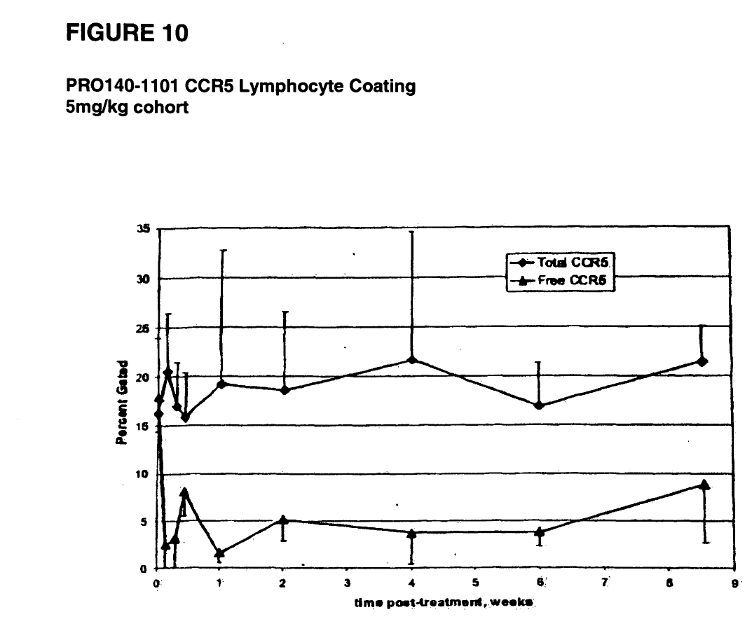

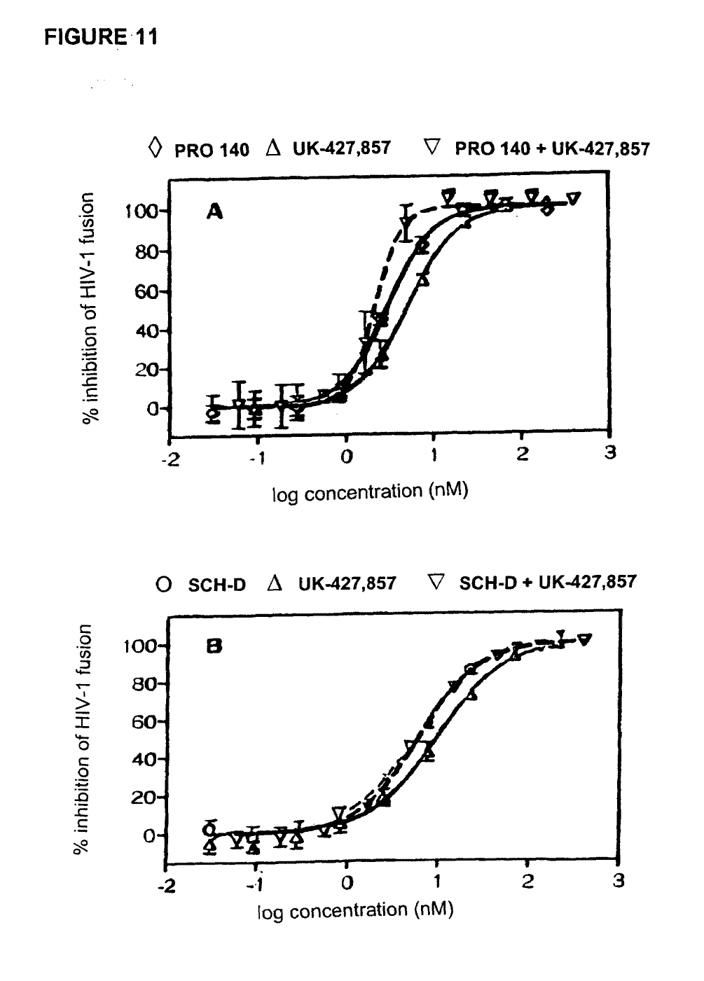

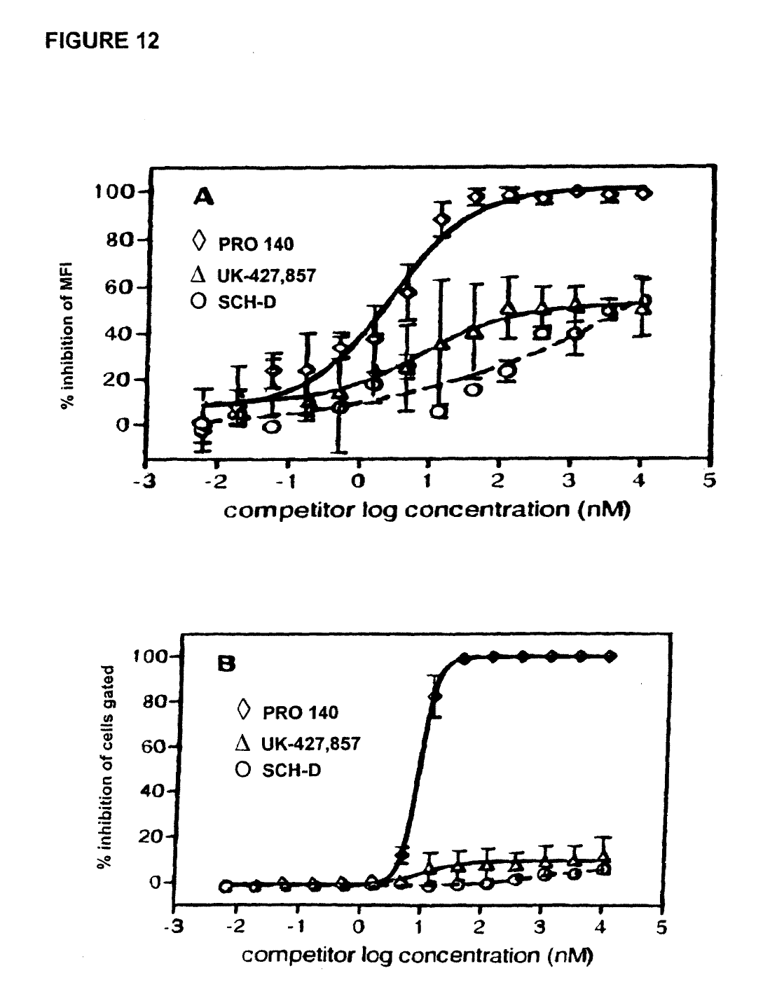

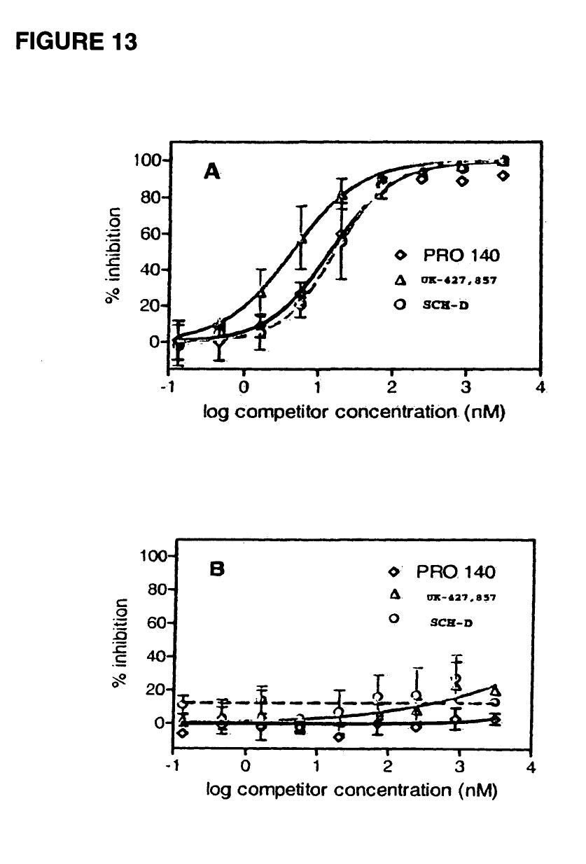

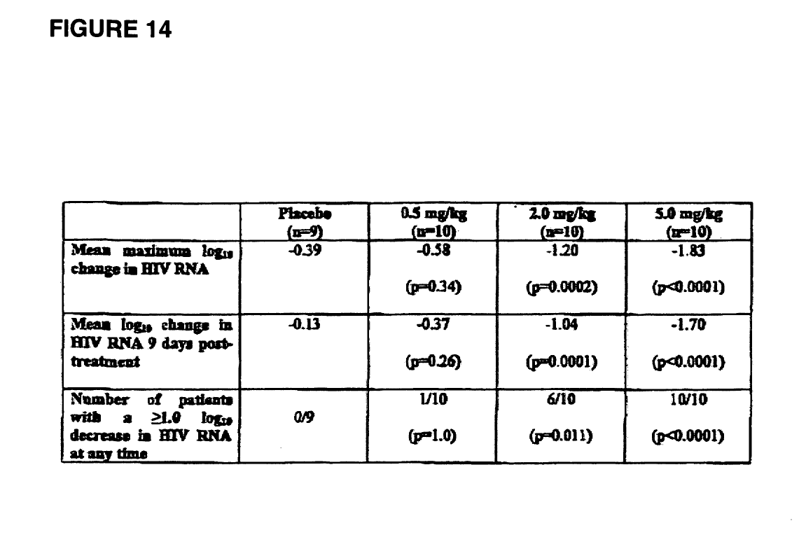

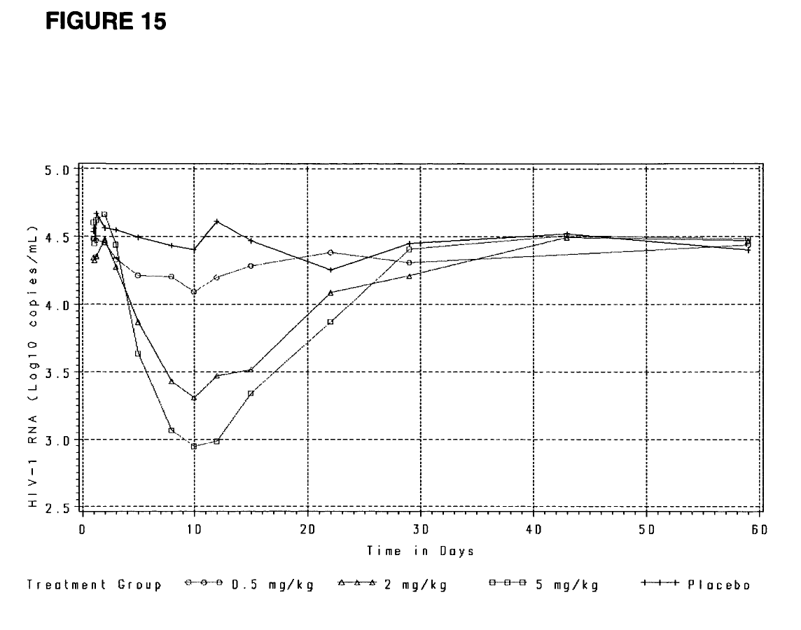

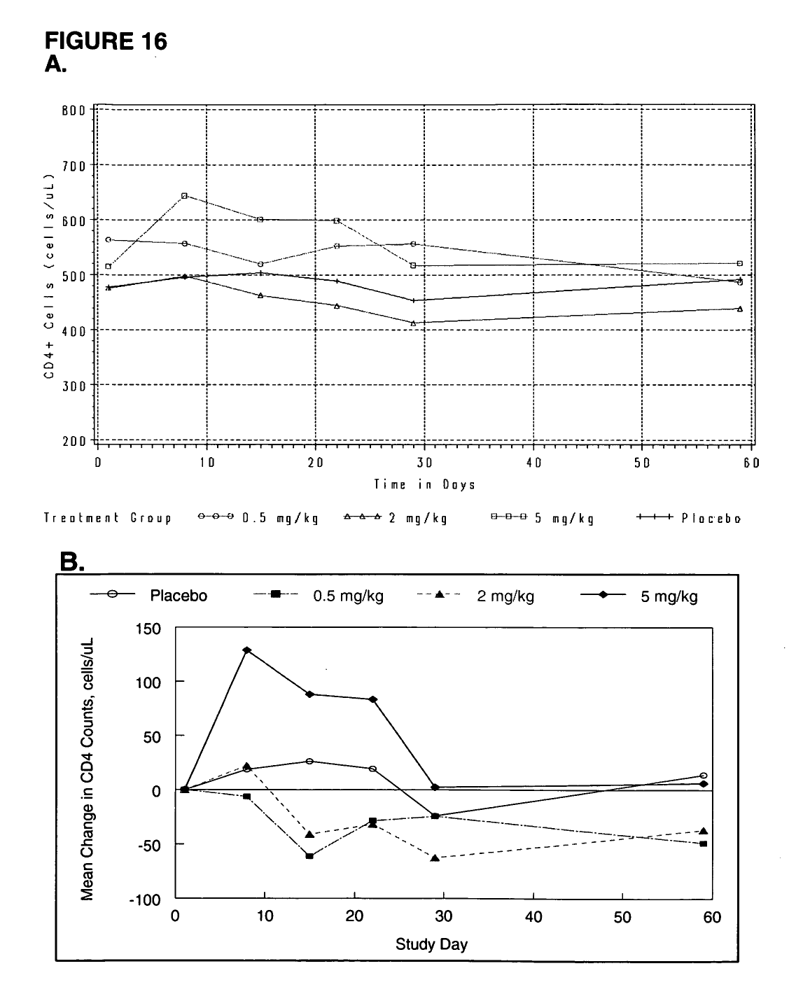

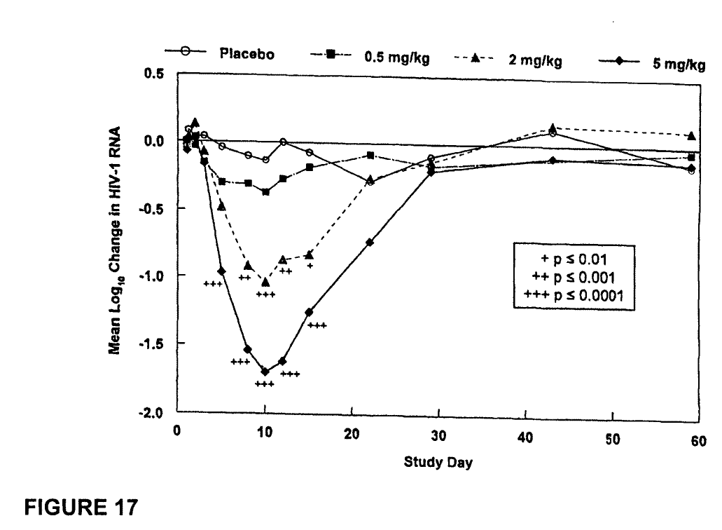

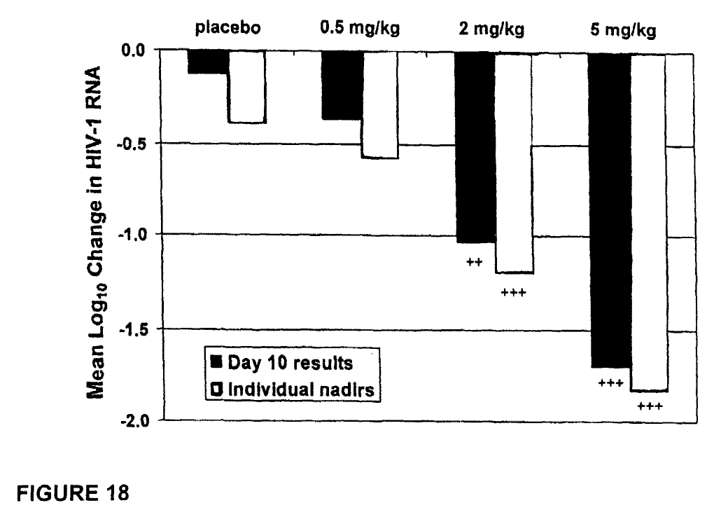

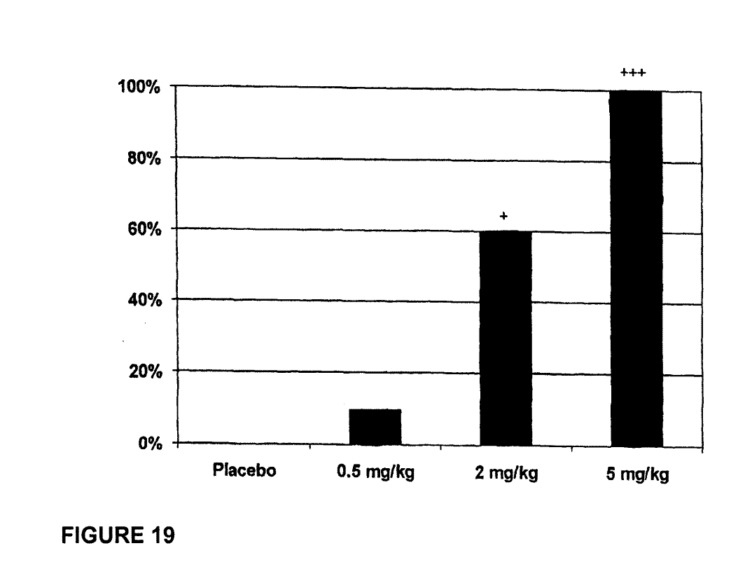

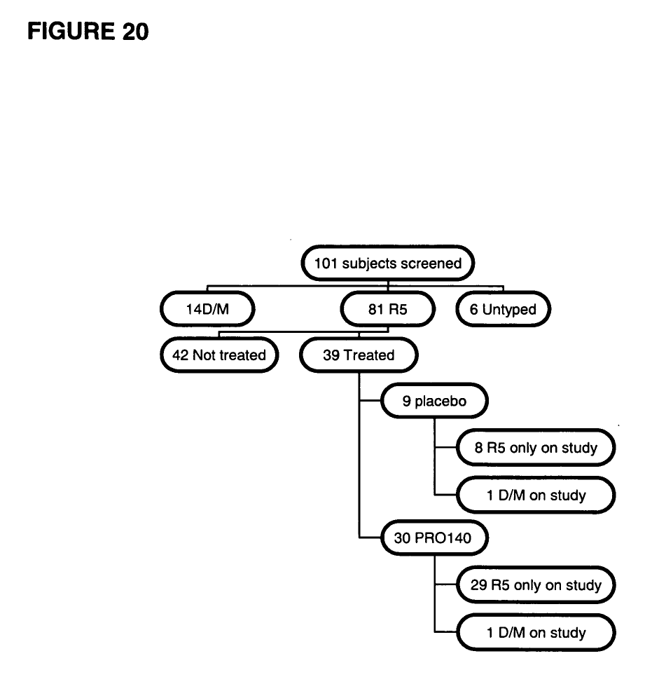

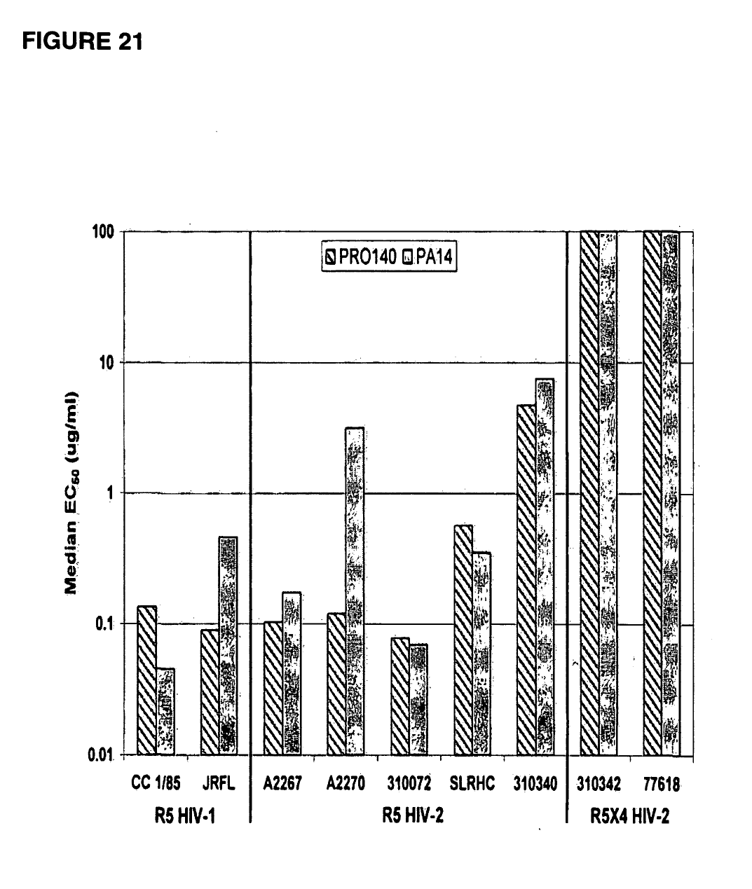

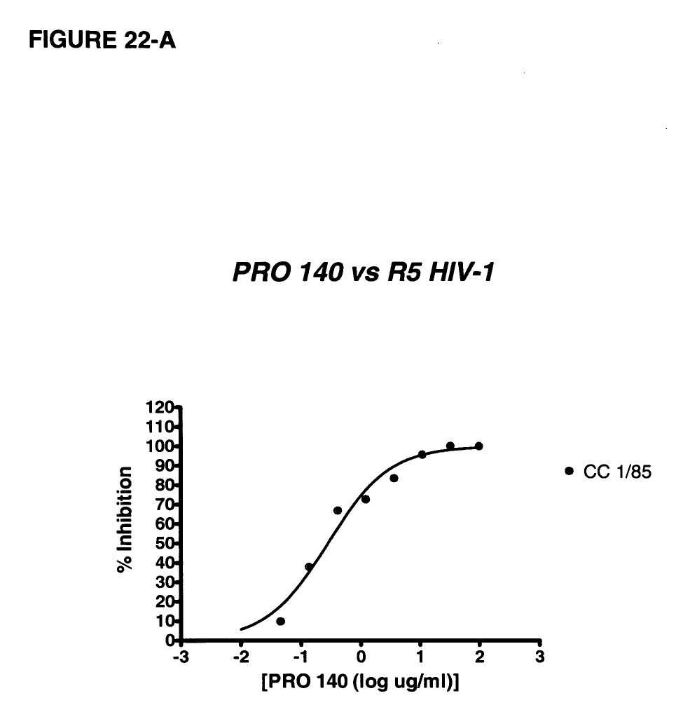

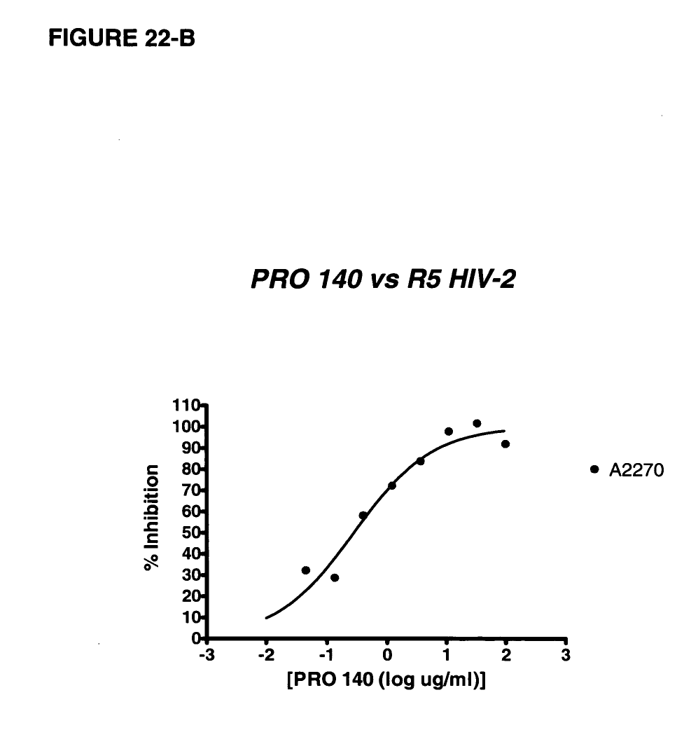

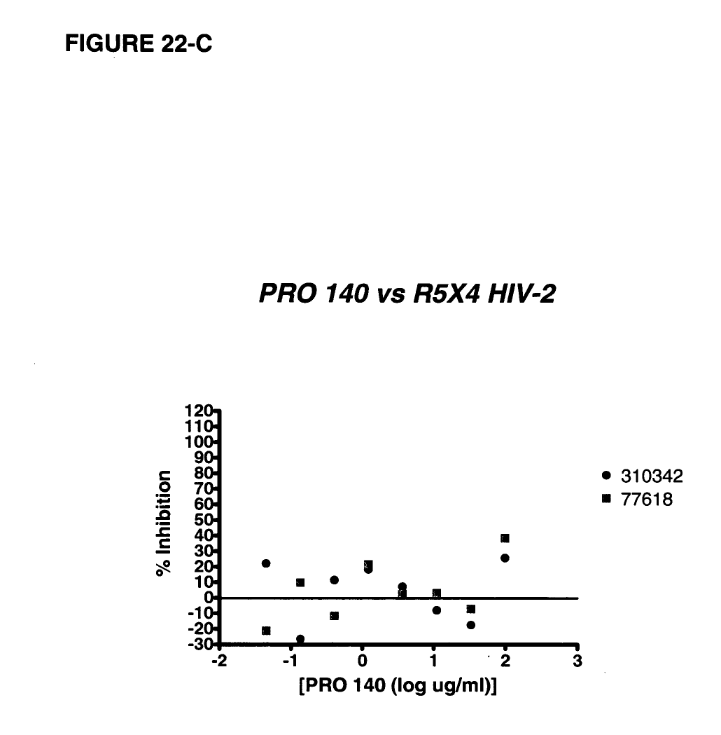

Healthy male volunteers were treated with a single intravenous infusion of 0.1 mg/kg PRO 140 (Cohort 1), 0.5 mg/kg PRO 140 (Cohort 2) or matched placebo. Plasma was collected post-treatment at the indicated times, cryopreserved and analyzed for levels of RANTES, a CC-chemokine that serves as a natural ligand for CCR5. RANTES levels were measured by ELISA in platelet-depleted plasma pre-dose and up to 28 days post-dose. As shown in FIG. 8, there was no significant change in RANTES levels following PRO 140 treatment (P>0.14 all times). These data are consistent with in vitro findings that PRO 140 does not antagonize CCR5 function. The findings suggest that PRO 140 does not have untoward effects on CCR5-mediated immune function in treated patients.

The results described herein indicate that in addition to PRO 140 broadly and potently inhibiting CCR5-mediated HIV-1 entry without CCR5 antagonism or other immunologic side effects in preclinical testing, this has demonstrated favorable tolerability, PK and immunologic profiles in preliminary results from an ongoing Phase 1a study in healthy volunteers. Thus, in many respects, PRO 140 offers a novel and attractive product profile for anti-HIV-1 therapy.

Moreover, the activities of anti-CCR5 mAbs are fundamentally distinct from, but complementary to, those of small-molecule CCR5 antagonists (see Table 2) which are also currently undergoing human clinical trials. PRO 140 has recently been shown to work synergistically with non-antibody CCR5 antagonists in inhibiting CCR5-mediated HIV-1 fusion to target cells. Accordingly, combination therapy comprising administration of anti-CCR5 mAbs and non-antibody CCR5 antagonists may offer powerfully effective, new approaches to preventing and treating HIV-1 infection.

### Part II

### Example 1

### Combination Testing of PRO 140 and HIV-1 Entry Inhibitors in the Fluorescence RET Assay

### Materials and Methods

Compounds and mAbs

PRO 140 was prepared by expression in Sp2/0 cells using Hybridoma serum-free medium supplemented with 2 mM L-glutamine (Invitrogen, Carlsbad, Calif.). Bulk mAb was clarified using a 5.0 μm Depth filter (Sartorius, Goettingen, Germany) followed by passage over a 0.2 μm sterilizing grade filter (Sartorius). The mAb was purified by passage first over an affinity column (MabSelect Protein A column, Amersham, Piscataway, N.J.) and then by ion exchange chromatography (SP Sepharose Cation Exchange resin, Amersham). PRO 140 was nanofiltered using a Viresolve™ 10 Opticap NFP capsule (Millipore, Billerica, Mass.) followed by a 0.2 μm filter and concentrated/diafiltered over disposable TFF cartridges (Millipore). The mAb was then polished over a hydroxyapatite column (Bio-Rad, Hercules, Calif.), concentrated to 10 mg/ml in phosphate-buffered saline and stored at −70° C. or colder prior to use. SCH-D (Schering Plough; Tagat et al., 2004), TAK-779 (Takeda Pharmaceuticals; Shiraishi et al., 2000), UK427,857 (Pfizer; Wood and Armour, 2005), and BMS378806 (Bristol-Myers Squibb; Lin et al., 2003) were prepared by commercial sources.

SCH-D has the following structure:

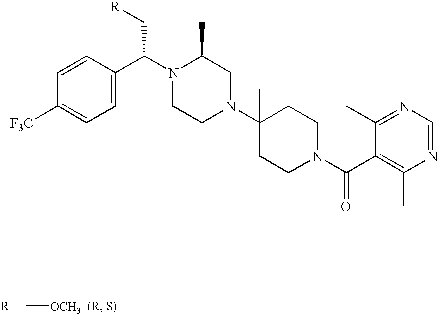

SCH-D (also designated SCH-417690): 1-[(4,6-dimethyl-5-pyrimidinyl)carbonyl]-4-[4-[2-methoxy-1(R)-4-(trifluoromethyl)phenyl]ethyl-3(S)-methyl-1-piperazinyl]-4-methylpiperidine (Schering-Plough)

SCH-D was synthesized according to the procedure described in Tagat et al. (2004) and set forth in FIG. 1.

TAK-779 has the following structure:

Y═—CH₂

X═—Cl

R¹═—CH₃

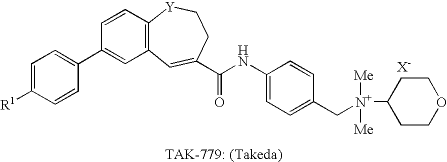

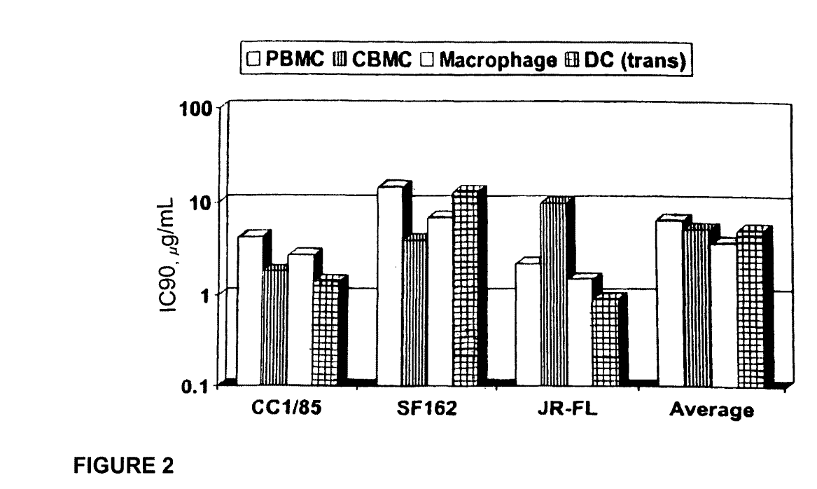

TAK-779 was synthesized according to the procedure described in Shiraishi et al. (2000) and set forth in FIG. 2.

TAK-652 has the following structure:

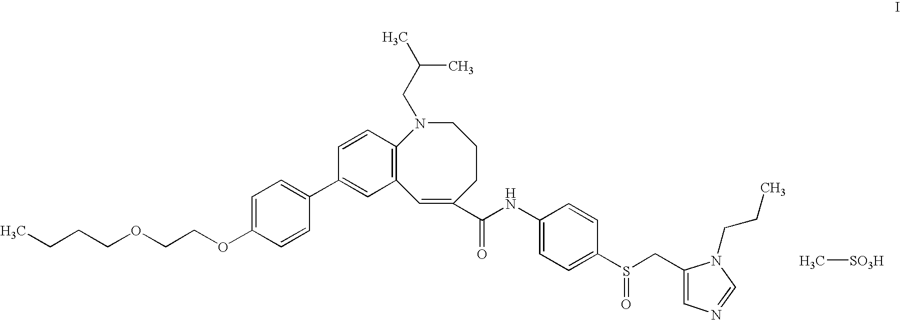

UK427,857 (maraviroc) has the following structure:

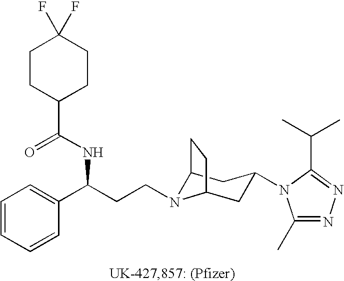

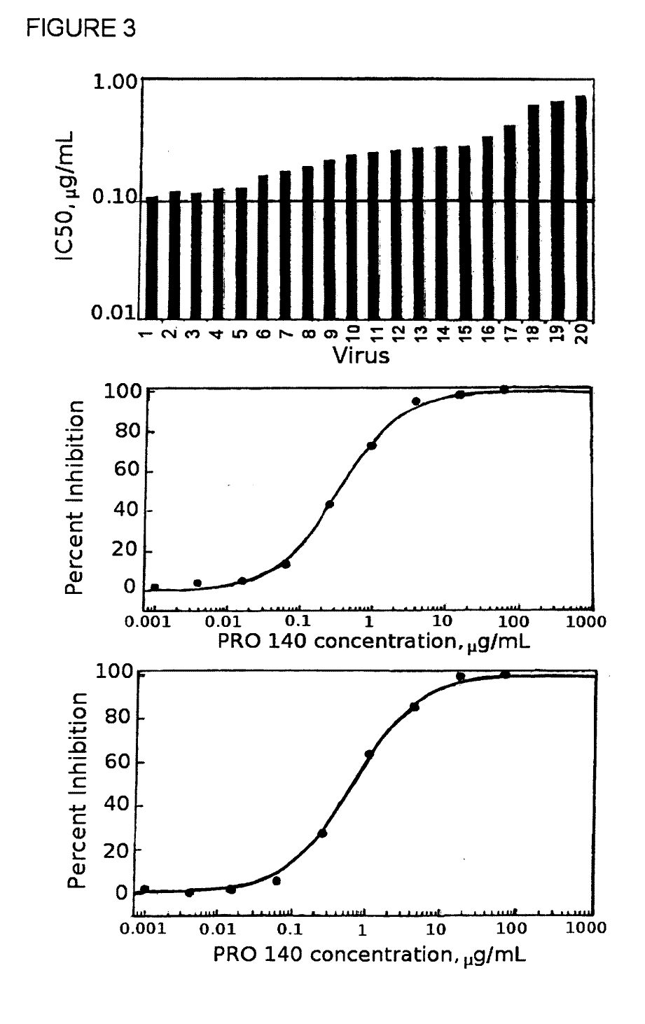

UK427,857 was synthesized according to the procedure described in PCT International Publication No. WO 01/90106 and set forth in FIG. 3.

BMS378806 has the following structure:

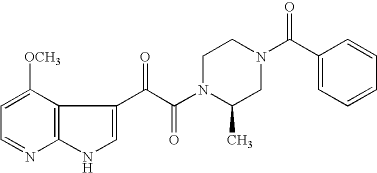

BMS378806: (R)-N-(benzoyl)-3-methyl-N′-[(4-methoxy-7-azaindol-3-yl)-oxoacetyl]-piperazine (Bristol-Myers Squibb)

It was synthesized according to the procedure described in U.S. Pat. No. 6,476,034 (compound 17a). Nevirapine (Boehringer Ingelheim; Merluzzi et al., 1990) and atazanavir (Bristol-Myers Squibb; Robinson et al., 2000) were purchased from commercial sources. PRO 542 was expressed in Chinese hamster ovary cells and purified as described previously (Allaway et al., 1995). T-20 (Fuzeon®) was synthesized by solid-phase fluoroenylmethoxycarbonyl chemistry, was purified by reverse-phase chromatography and was analyzed for purity and size by HPLC and mass spectroscopy as described previously (Nagashima et al., 2001). AZT was purchased from Sigma Chemicals (St. Louis, Mo.). RANTES was purchased from R&D Systems (Minneapolis, Minn.). The anti-CCR5 mAb 2D7 was purchased from Pharmingen (San Diego, Calif.), and the anti-CD4 mAb Leu-3A was purchased from Becton Dickinson (Franklin Lakes, N.J.).

For testing, small molecule compounds were solubilized in dimethylsulfoxide (DMSO) to 10 mM and then diluted in DMSO to 200× the final concentration to be utilized in the antiviral assay. Serial dilutions of small molecules were conducted in DMSO. Subsequent dilutions were conducted in medium to achieve a final DMSO concentration in the assay of 0.5%. Peptides and mAbs were diluted in PBS in the absence of DMSO. Typically, inhibitor concentrations in the RET assay included eleven 3-fold dilutions ranging from 200 nM to 3.0 μM.

### Cell Preparation

HeLa cells were engineered to express HIV-1 gp120/gp41 from the macrophage-tropic primary isolate JRFL as described (HeLa-Env_{JRFL}; Litwin et al., 1996). Briefly, the HIV-1_{LAI} Env gene was excised from the plasmid pMA243 (Dragic et al., 1992) and the HIV-1_{JRFL} Env gene was inserted. The HIV-1_{JRFL} Env gene was amplified from the plasmid pUCFL112-1 (Koyanagi et al., 1987). The resulting plasmid, designated JR-FL-pMA243, was sequenced by standard methods and transfected into HeLa cells using lipofectin (Gibco BRL/Invitrogen, Carlsbad, Calif.). HeLa-Env_{JRFL} transfectants were selected in methotrexate (Sigma, St. Louis, Mo.) and cloned twice by limiting dilution. The transduced human T cell leukemia line CEM NKR-CCR5 cells were obtained from the NIH AIDS Research and Reference Program (Cat. No. 458).

### RET Assay

The HIV-1 RET assay has been described in detail previously (Litwin et al., 1996). Briefly, fluorescein octadecyl ester (F18; Molecular Probes, Eugene, Oreg.; 5 mg/ml in ethanol), was diluted 1:800 in DMEM labeling medium (DMEM; Invitrogen, Carlsbad, Calif.) with 10% fetal bovine serum (FBS; HyClone, Logan, Utah) and adjusted to an A₅₀₆ of 0.34±10%. Octadecyl rhodamine B chloride (R18; Molecular Probes; 10 mg/ml in ethanol) was diluted 1:2050 in labeling medium and adjusted to an A₅₆₅ of 0.52±10%. Both dyes were further diluted 2-fold by addition to cells in T75-cm² flasks. HeLa-Env_{JRFL} and CEM NKR-CCR5 cells were incubated overnight in F18- and R18-containing culture medium, respectively. The following day, medium from HeLa-Env_{JRFL} cells was removed and 10 ml of 0.5 mM EDTA was added and incubated at 37° C. for 5 min. EDTA was removed and the flask was returned to the incubator for another 5 min followed by striking of the flask to dislodge cells. Ten ml of PBS- with 15% FBS were added to the flask and the contents were transferred to a 50-ml conical centrifuge tube. Suspension CEM NKR-CCR5 cells were added directly to a separate 50-ml conical centrifuge tube. Both cell lines were centrifuged at 300×g for 5 min. The supernatant was discarded and cells were resuspended in 10 ml of PBS-/15% FBS. The centrifugation/wash step was repeated twice, after which the cells were counted and concentrations adjusted to 1.5×10⁶ cells/ml. Ten μl of each cell type (15,000 cells) were seeded into wells of a 384-well plate. Inhibitor compounds were added immediately thereafter to bring the final well volume to 40 μl, and the plates were incubated for 4 h at 37° C. Compounds were tested individually and in combination at a fixed molar ratio or mass ratio over a range of serial dilutions. The plates were then read on a fluorescence plate reader (Victor2, Perkin Elmer, Boston, Mass.) using the excitation/emission filter combinations shown in Table 6.

TABLE 6
Excitation/emission filter combinations for RET assay

| Scan No. | Excitation wavelength | Emission wavelength |
|---|---|---|
| 1 | 450 nm/50 nm | 530 nm/25 nm |
| 2 | 530 nm/25 nm | 590 nm/35 nm |
| 3 | 450 nm/50 nm | 590 nm/35 nm |

The “% RET” was calculated according to the following formula after subtraction of background (blank) readings:

% RET=100×[(A₃−(A₁×Fₛₚᵢₗₗ)−(A₂×Rₛₚᵢₗₗ))/A₂]

### Where:

Fₛₚᵢₗₗ=HeLa cells alone, Scan 3/Scan 1;
Rₛₚᵢₗₗ=CEM cells alone, Scan 3/Scan 2;
A₁=Scan 1 value for HeLa and CEM cells in combination;
A₂=Scan 2 value for HeLa and CEM cells in combination; and
A₃=Scan 3 value for HeLa and CEM cells in combination.

The “% Inhibition” was calculated according to the following formula:

% Inhibition=100×[(Max % RET−% RET for sample well)/(Max % RET−Min % RET)]

### Where:

Max % RET=average of % RET values for HeLa and CEM cell combination without added inhibitor; and
Min % RET=average of % RET values for HeLa and CEM cell combination in presence of 500 ng/ml of Leu-3a mAb (an antibody that targets CD4 and fully blocks fusion in the RET assay at this concentration).

Fifty percent inhibition (IC₅₀) values were determined by fitting the inhibition data with a non-linear, four-parameter, variable slope equation (GraphPad Prism, ver. 4.02; GraphPad Software, San Diego, Calif.). Upper and lower inhibition values were constrained to 100% and 0%, respectively for curve fitting.

### Synergy Determinations

Cooperative inhibition effects of drug combinations were determined by the method of Chou and Talalay (1984). IC₅₀ values were generated for all combinations as described above. Combination Index (CI) and Dose Reduction (DR) values were calculated according to the following formulas:

$$C l = ( \frac{I {C}_{50} D comb 1}{I {C}_{50} D solo 1} ) + ( \frac{I {C}_{50} D comb 2}{I {C}_{50} D solo 2} ) + α ( \frac{( I {C}_{50} D comb 1 ) ( I {C}_{50} D comb 2 )}{( I {C}_{50} D solo 1 ) ( I {C}_{50} D solo 2 )} )$$

DR (for compound 1)=(IC₅₀ Dsolo1/IC₅₀ Dcomb1)

DR (for compound 2)=(IC₅₀ Dsolo2/IC₅₀ Dcomb2)

### Where:

“IC₅₀ Dcomb1”=IC₅₀ of drug 1 in combination with drug 2;
“IC₅₀ Dsolo1”=IC₅₀ of drug 1 when tested alone;
“IC₅₀ Dcomb2”=IC₅₀ of drug 2 in combination with drug 1;
“IC₅₀ Dsolo2”=IC₅₀ of drug 2 when tested alone;
α=0 if the effects of the two drugs are mutually exclusive; and
α=1 if the effects of the two drugs are mutually nonexclusive

Combinations with CI<1 are determined to be synergistic, whereas combinations with CI>1 are determined to be antagonistic. Additivity is reflected in combinations for which CI=1.

Ninety five percent Confidence Intervals were calculated in Microsoft Excel using the formula:

=Confidence(alpha,stdev,n)

### Where:

alpha=0.05 (95% confidence);
stdev=standard deviation of dataset mean; and
n=number of replicates.

### Results

### Preparation of Small-Molecule Fusion Inhibitors

SCH-D, TAK-779, UK427,857, and BMS378806 were prepared by commercial sources. The desired quantities and HPLC purity of the compounds were realized. Purity of the compounds was supported by results obtained from elemental analysis, and the identities of the products were confirmed by proton NMR (proton and carbon-13) and/or mass spectrum data.

### Synergistic Interactions Revealed by RET Assay

Synergy experiments were conducted using the cell-cell RET fusion assay to assess initially the potential for cooperative interactions between PRO 140 and small-molecule and peptide-based inhibitors of CCR5, CD4, HIV-1 gp120 and HIV-1 gp41. The experiments were then extended to the CCR5-specific murine monoclonal antibody, 2D7 (Wu et al., 1997).

Experiments measuring inhibition of HIV-1 Env-mediated fusion were first conducted using combinations of PRO 140 with, respectively, PRO 140 itself, 3 small-molecule CCR5 antagonists (SCH-D, TAK-779, UK427857), the natural peptide ligand of CCR5 (RANTES), and an anti-CCR5 mAb (2D7), a peptide-based inhibitor of gp41 (T-20), a protein-based inhibitor of gp120 (PRO 542), a small-molecule inhibitor of gp120 (BMS378806), and an anti-CD4 mAb (Leu3A). Mass ratios of PRO 140 to other entry inhibitors ranged from 0.75 to 364. The results are shown in Table 7.

TABLE 7
Combination Index and Dose Reduction Values for inhibition of HIV-1 Env-mediated
fusion with combinations of PRO 140 and entry inhibitors

| | | | | | Mean Dose | Mean Dose |
|---|---|---|---|---|---|---|
| PRO 140 in | | | | | Reduction | Reduction (Cpd |
| combination | No. of | Cpd mass | Inhibitor | Mean CI^{c} | (PRO 140) | in combination) |
| with:^{a} | tests | ratios^{b} | target | Cell-cell fusion assay | | |
| PRO 140 | 9 | 1 | CCR5 | 0.97 ± 0.08 | 2.07 ± 0.18 | 2.07 ± 0.18 |
| TAK-779 | 8 | 282 | CCR5 | 0.36 ± 0.10 | 4.10 ± 2.03 | 15.86 ± 7.10 |
| SCH-D | 9 | 279 | CCR5 | 0.51 ± 0.05 | 4.21 ± 0.96 | 3.90 ± 0.71 |
| UK-427,857 | 3 | 292 | CCR5 | 0.59 ± 0.04 | 4.16 ± 0.41 | 2.98 ± 0.65 |
| RANTES | 4 | 19 | CCR5 | 0.59 ± 0.08 | 4.13 ± 0.99 | 3.24 ± 1.06 |
| 2D7 | 2 | 1 | CCR5 | 0.93 ± 0.04 | 1.87 ± 0.07 | 2.54 ± 0.13 |
| T-20 | 7 | 33 | gp41 | 0.84 ± 0.16 | 1.77 ± 0.40 | 7.47 ± 3.34 |
| PRO 542 | 6 | 0.75 | gp120 | 0.96 ± 0.17 | 1.59 ± 0.21 | 5.54 ± 1.49 |
| BMS-378806 | 7 | 364 | gp120 | 1.21 ± 0.21 | 1.64 ± 0.30 | 2.85 ± 0.76 |

^{a}Compounds were tested at a 1:1 molar ratio.
^{b}Mass of PRO 140/mass of other HIV-1 entry inhibitor tested in combination. Molecular weights of inhibitors are: PRO 140 ≈ 150,000 g/mole; SCH-D = 538 g/mole; TAK-779 = 531 g/mole (hydrochloride salt); UK-427,857 = 514 g/mole; RANTES ≈ 7,800 g/mole; 2D7 ≈ 150,000 g/mole; T-20 = 4,492 g/mole; PRO 542 ≈ 200,000 g/mole; BMS-378806 = 412 g/mole.
^{c}Combination Index at IC₅₀ value. The mutually exclusive CI formula (α = 0) was utilized for PRO 140 in combination with molecules that bind CCR5, and the mutually non-exclusive formula (α = 1) was utilized for PRO 140 in combination with molecules that bind other targets (Chou and Rideout, 1991).

Two small-molecule CCR5 antagonists, SCH-D and TAK-779, were assayed in combination. PRO 542, a recombinant antibody-like fusion protein in which the heavy- and light-chain variable domains of human IgG2 have been replaced with the D1D2 domains of human CD4, was also tested in combination with the anti-CD4 mAb, Leu-3A. The results of these assays are shown in Table 8.

TABLE 8
Other drug combinations tested in the RET assay for cooperativity

| | | Molar ratios | | Mean CI ± | Mean DR | Mean DR |
|---|---|---|---|---|---|---|
| Drug 1 | Drug 2 | (Drug 1 to 2) | N | stdev^{a} | (Drug 1) | (Drug 2) |
| SCH-D | TAK-779 | 1:1 | 4^{b} | 1.12 ± 0.32 | 1.48 ± 0.96 | 4.31 ± 1.82 |
| PRO 542 | Leu-3A | 22.9:1 | 2 | 16.9 ± 0.3 | 0.7 ± 0 | 0.16 ± 0 |

^{a}CI values were calculated using the mutually exclusive formula for SCH-D vs. TAK-779 (i.e., where α = 0) and the mutually non-exclusive formula for PRO 542 vs. Leu-3A (i.e., where α = 1; see methods).
^{b}One aberrant datapoint was culled from the calculation of Mean CI and Mean DRs.

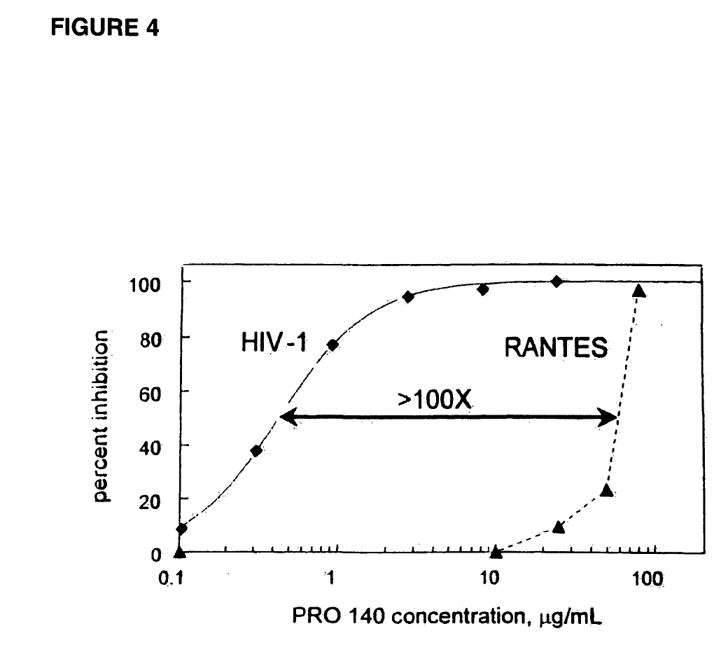

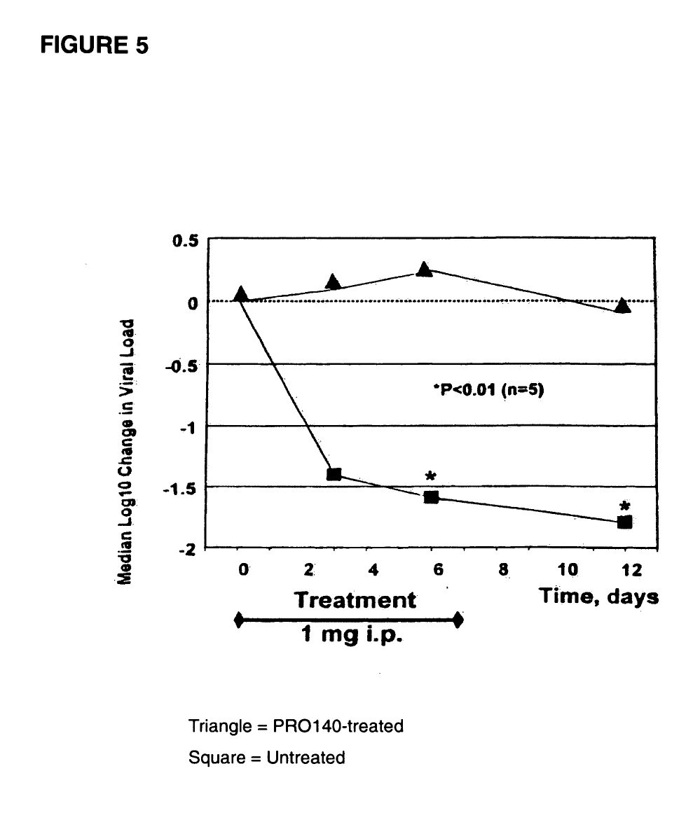

The effect of varying the relative amounts of compounds in the combinations on the level of cooperativity was also measured. Molar ratios of 5:1 and 1:5 PRO 140 were used. The results are tabulated in Table 9, and the mean CI values with 95% confidence intervals are plotted in FIG. 4 for the 1:1 molar ratio data. In addition to PRO 140, the inhibitory activity of mAb 2D7, a CCR5-specific murine antibody (Wu et al., 1997) was also tested in combination with the small-molecule CCR5 antagonists and with RANTES using the fluorescent RET assay. The results are shown in Table 10.

TABLE 9
Combination Index and Dose Reduction Values for inhibition of HIV-1 Env-mediated
fusion with combinations of PRO 140 and entry inhibitors

| | | | Mean | Mean Dose | |
|---|---|---|---|---|---|
| PRO 140 in | | | Combination | Reduction | Mean Dose Reduction |
| combination | | Cpd Mass | Index^{c} | (PRO 140) | (Cpd. in combination) |
| with: | Ratio^{a} | Ratios^{b} | Cell-cell fusion assay | | |
| PRO 140 | 5:1 | 5 | 1.15 ± 0.09 | 1.05 ± 0.08 | 5.26 ± 0.41 |
| PRO 140 | 1:5 | 0.2 | 1.09 ± 0.08 | 5.54 ± 0.38 | 1.10 ± 0.08 |
| TAK-779 | 5:1 | 1410 | 0.57 ± 0.07 | 1.89 ± 0.14 | 33.59 ± 18.85 |
| TAK-779 | 1:5 | 56.4 | 0.52 ± 0.20 | 5.58 ± 0.52 | 3.78 ± 1.95 |
| SCH-D | 5:1 | 1395 | 0.66 ± 0.10 | 1.92 ± 0.40 | 8.44 ± 1.27 |
| SCH-D | 1:5 | 55.8 | 0.69 ± 0.05 | 9.95 ± 2.03 | 1.73 ± 0.19 |
| UK-427,857 | 5:1 | 1460 | 0.66 ± 0.11 | 2.00 ± 0.35 | 7.25 ± 2.19 |
| UK-427,857 | 1:5 | 58.4 | 0.73 ± 0.05 | 11.31 ± 2.14 | 1.58 ± 0.17 |
| RANTES | 5:1 | 95 | 0.84 ± 0.14 | 1.63 ± 0.43 | 5.39 ± 1.13 |
| RANTES | 1:5 | 3.8 | 0.66 ± 0.06 | 13.64 ± 4.75 | 1.75 ± 0.28 |
| T-20 | 5:1 | 165 | 1.10 ± 0.12 | 0.98 ± 0.11 | 31.85 ± 10.19 |
| T-20 | 1:5 | 6.6 | 0.76 ± 0.27 | 2.93 ± 0.68 | 3.85 ± 1.50 |
| PRO 542 | 5:1 | 3.75 | 1.13 ± 0.10 | 1.01 ± 0.07 | 15.73 ± 4.15 |
| PRO 542 | 1:5 | 0.15 | 1.18 ± 0.17 | 2.83 ± 0.50 | 1.71 ± 0.29 |
| BMS-378806 | 5:1 | 1820 | 1.12 ± 0.10 | 1.14 ± 0.06 | 8.88 ± 4.16 |
| BMS-378806 | 1:5 | 72.8 | 1.55 ± 0.24 | 3.64 ± 0.73 | 1.07 ± 0.31 |

^{a}Molar ratio of PRO 140 to other entry inhibitor tested in combination (n = 3 for all experimental results)
^{b}Mass of PRO 140/mass of other HIV-1 entry inhibitor tested in combination. Molecular weights of inhibitors are: PRO 140 ≈ 150,000 g/mole; SCH-D = 538 g/mole; TAK-779 = 531 g/mole (hydrochloride salt); UK-427,857 = 514 g/mole; RANTES ≈ 7,800 g/mole; T-20 = 4,492 g/mole; PRO 542 ≈ 200,000 g/mole; BMS-378806 = 412 g/mole.
^{c}Combination Index at IC₅₀ value. The mutually exclusive CI formula (α = 0) was utilized for PRO 140 in combination with molecules that bind CCR5, and the mutually non-exclusive formula (α = 1) was utilized for PRO 140 in combination with molecules that bind other targets (Chou and Rideout, 1991).

TABLE 10
Combination Index and Dose Reduction Values for inhibition of HIV-1 Env-mediated
fusion with combinations of 2D7 and entry inhibitors

| | | | Mean | Mean Dose | Mean Dose |
|---|---|---|---|---|---|
| 2D7 in | | | Combination | Reduction | Reduction (Cpd |
| combination | Cpd Mass | Inhibitor | Index^{b} | (2D7) | in combination) |
| with:^{a} | Ratios^{c} | target | Cell-cell fusion assay | | |
| TAK-779 | 282 | CCR5 | 0.15 ± 0.03 | 17.20 ± 3.23 | 11.95 ± 4.94 |
| SCH-D | 279 | CCR5 | 0.57 ± 0.10 | 3.25 ± 0.56 | 4.04 ± 0.78 |
| UK427857 | 292 | CCR5 | 0.58 ± 0.03 | 2.45 ± 0.12 | 5.73 ± 0.54 |
| RANTES | 19 | CCR5 | 0.62 ± 0.04 | 1.94 ± 0.08 | 10.18 ± 1.86 |
| PRO 140 | 1 | CCR5 | 0.93 ± 0.04 | 2.54 ± 0.13 | 1.87 ± 0.07 |

^{a}Compounds were tested at a 1:1 molar ratio (all data are n = 3 except for 2D7 and PRO 140, where n = 2)
^{b}Combination Index at IC₅₀ value. The mutually exclusive CI formula (α = 0) was utilized for 2D7 in combination with molecules that bind CCR5 (Chou and Rideout, 1991).
^{c}Mass of 2D7/mass of other HIV-1 entry inhibitor tested in combination. Molecular weights of inhibitors are: 2D7 ≈ 150,000 g/mole; SCH-D = 538 g/mole; TAK-779 = 531 g/mole (hydrochloride salt); UK-427,857 = 514 g/mole; RANTES ≈ 7,800 g/mole.

### Example 2

### Combination Testing of PRO 140 with Small Molecule, Peptide and Protein Inhibitors, and HIV-1 in the HIV-1 Pseudovirus Particle (HIV-1pp) Assay

### Materials and Methods

### Preparation of HIV-1 Pseudoparticles

HIV-1 pseudoparticles (HIV-1pp) are generated in 293T cells by transient coexpression of an HIV-1-based NL4/3luc+env− plasmid and a construct encoding HIV-1_{JRFL} Env. The NL4/3luc+env− plasmid was obtained from the NIH AIDS Research and Reference Reagent Program (Cat. No. 3418), and the HIV-1_{JRFL} Env was inserted into the pcDNA3.1 vector (Invitrogen). Briefly, 293T cells are calcium phosphate transfected with a 1:1 ratio of NL4/3luc+env− reporter vector and Env expression vector in Hepes buffer (Profection Mammalian Transfection Kit, Promega). After 16 h the transfection medium is aspirated and fresh cell culture medium (DMEM with 10% FBS, glutamine and antibiotics) is added and the incubation is continued at 37° C. for an additional 24-32 h. Cell culture supernatants are collected 48 h post-transfection and centrifuged at 1,400 rpm for 10 min to pellet cell debris. The viral supernatant is brought to a final concentration of 5% sucrose and stored aliquoted at −80° C.

### Cells

U87-CD4-CCR5 cells were obtained from the NIH AIDS Research and Reference Program (Cat. No. 4035). These cells are maintained in culture medium (DMEM with 10% FBS, antibiotics and glutamine) containing 0.3 mg/ml G418 and 0.5 mg/ml puromycin. Cells are grown in T175-cm² flasks at 37° C. and diluted 1:5 every 3-4 days. For assay plate preparation, cells are trypsinized and seeded into wells of 96-well tissue-culture treated flat bottom opaque polystyrene plates (Perkin Elmer, Boston, Mass.) at a density of 3×10³ cells/well. Plates are incubated for no more than 4 h at 37° C. in a humidified 5% CO₂ incubator prior to their use in the HIV-1 pp susceptibility assay.

### Compound Preparation

Fifty μl of diluted compound at 4× the desired final concentration are added per well. For compounds solubilized in DMSO, the 4× stock will contain 2% DMSO (such that the final DMSO concentration in the assay is always 0.5% for small molecules). Control wells receiving no compound are included on each plate. In addition, an AZT inhibition control is included in each assay. Compounds are tested individually and at a fixed mass or molar ratio over a broad range of concentrations.

### Virus Addition

A vial of frozen, aliquoted HIV-1pp is thawed in a 37° C. waterbath and then placed on wet ice. Virus is diluted in cold cell culture medium as necessary to achieve the desired final virus concentration in the HIV-1pp assay (about 10,000 relative light units (rlu) per well). 50 μl of diluted virus are added per well, bringing the final well volume to 200 μl. A no-virus control (minimum or background luminescence) and a no-compound control (maximum luminescence) are included on each plate. The plates are incubated for 72 h at 37° C. in a humidified 5% CO₂ incubator followed by processing for luciferase signal (see below).

### Plate Processing for Luciferase Assay

Assay medium is aspirated and 200 μl of PBS are added to each well. The PBS is aspirated and 50 μl of 1× Cell Lysis Reagent (Promega—Cat. No. E1531) are added to each well. Assay plates are then frozen for at least 2 h at −80° C. followed by thawing at room temperature and vigorous mixing with an electronic pipettor. 25 μl from each well are transferred to an opaque 96-well plate (Costar #3922). Four replicates are pooled into the same well on the opaque plate. 100 μl of freshly thawed and reconstituted luciferase substrate (Luciferase Assay System, Promega—Cat. No. E1501) are added to each well of the plate with the electronic pipettor, and luminescence is detected immediately on a Dynex MLX plate reader set to medium gain.

### Data Analysis

Neutralization activity is displayed by plotting the percent inhibition of luciferase activity (after background rlu values are subtracted from all datapoints) versus log₁₀ drug concentration. The percent inhibition is derived as follows: [1−(luciferase activity in the presence of drug/luciferase activity in the absence of drug)]×100. IC₅₀ values are determined by fitting the inhibition data with a non-linear, four-parameter, variable slope equation (GraphPad Prism, ver. 4.02; GraphPad Software, San Diego, Calif.). Upper and lower inhibition values are constrained to 100% and 0%, respectively for curve fitting.

### Synergy Determination

Cooperative interactions between PRO 140 and small-molecule and peptide-based inhibitors of CCR5, CD4, HIV-1 gp120, HIV-1 gp41 and HIV-1 reverse transcriptase (see Tables 4 and for listing of HIV-1 inhibitors approved for clinical use) are determined as described in Example 1. Cooperative inhibition effects of drug combinations are determined by the method of Chou and Talalay (1984). IC₅₀ values are generated for all combinations as described above. Combination Index (CI) and Dose Reduction (DR) values are calculated according to the following formulas:

$$C l = ( \frac{I {C}_{50} D comb 1}{I {C}_{50} D solo 1} ) + ( \frac{I {C}_{50} D comb 2}{I {C}_{50} D solo 2} ) + α ( \frac{( I {C}_{50} D comb 1 ) ( I {C}_{50} D comb 2 )}{( I {C}_{50} D solo 1 ) ( I {C}_{50} D solo 2 )} )$$

DR (for compound 1)=(IC₅₀ Dsolo1/IC₅₀ Dcomb1)

DR (for compound 2)=(IC₅₀ Dsolo2/IC₅₀ Dcomb2)

### Where:

“IC₅₀ Dcomb1”=IC₅₀ of drug 1 in combination with drug 2;
“IC₅₀ Dsolo1”=IC₅₀ of drug 1 when tested alone;
“IC₅₀ Dcomb2”=IC₅₀ of drug 2 in combination with drug 1;
“IC₅₀ Dsolo2”=IC₅₀ of drug 2 when tested alone;
α=0 if the effects of the two drugs are mutually exclusive; and
α=1 if the effects of the two drugs are mutually nonexclusive.

Combinations with CI<1 are determined to be synergistic, whereas combinations with CI>1 are determined to be antagonistic. Additivity is reflected in combinations for which CI=1.

### Example 3

### Combination Testing of PRO 140 with Small Molecule, Peptide and Protein Inhibitors in the HIV-1 Authentic Virus Replication Assay

### Materials and Methods

### Preparation of PBMCs

Replication of authentic HIV-1 is measured in activated peripheral blood mononuclear cells (PBMCs) using the monocyte/macrophage-tropic HIV-1 clone, JRFL (HIV-1_{JRFL}), for these studies.

PBMCs are isolated from 4 separate donors (Leukopacks) by centrifugation on a Ficoll gradient. CD8 cells are depleted using RosetteSep CD8 Depletion Cocktail (#15663, StemCell Research, Vancouver, BC). Cells are diluted to 4×10⁶/ml and added in equal parts to three T175-cm² flasks and then stimulated by addition of one of the following media: IL-2 Medium [RPMI 1640 (#10-040-CV, Cellgro, Herndon, Va.), 10% FBS (#35-010-CV), 2 mM L-Glutamine (#25-005-CI), 100 U/ml IL-2 (Sigma, St. Louis, Mo.)]; PHA 5 Medium: [IL-2 Medium with 5 ug/ml Phytohemagglutinin PHA-P (PHA) (#L8754, Sigma, St. Louis, Mo.), filtered]; or PHA 0.5 Medium: [IL-2 Medium with 0.5 ug/ml PHA, filtered]. Each flask receives a total of 50-150 ml of medium. Flasks are incubated for 3 days at 37° C. followed by pooling of the contents prior to use in the infection assay.

### Virus Titration

Serial dilutions of virus are tested in quadruplicate on activated PBMCs (1.4×10⁵ PBMC/well). Titration Medium [IL-2 Medium with 100 IU/ml penicillin/streptomycin (#30-002-CI, Cellgro)] is utilized for virus titrations. Fifty μl of diluted virus is added to 100 μl of PBMCs in flat bottom, tissue-culture treated 96-well plates (VWR# 29442-054, Corning, Corning, N.Y.) and the plates are incubated at 37° C. in a humidified, 5% CO₂ incubator. After 7 days, 50 μl are removed from each well and tested for virus levels by p24 antigen ELISA (Perkin Elmer, Boston, Mass.). Virus titer is determined by the method of Reed and Muench (Table 11).

### Neutralization Assay

Stimulated PBMCs are seeded into wells of 96-well flat bottom plates at a density of 1.4×10⁵ cells/well. Virus is diluted to 2,000 TCID₅₀/ml and mixed with serial 0.5 log₁₀ dilutions of compound for 1 h at 37° C. prior to addition to the cell plates. The final amount of virus added per well is 100 TCID₅₀. The final DMSO concentration in the assay is always 0.5% whenever small molecule inhibitors are being tested. Plates are incubated at 37° C. for 5 days, at which time an aliquot of supernatant is removed for p24 antigen ELISA. If control wells (virus without inhibitor) exhibit low p24 antigen levels then the plates are brought back to full volume with Titration medium and incubated for an additional 24 h.

TABLE 11
Reed and Muench formula for calculating virus titer^{a}

| No. of | |
|---|---|
| pos. | TCID₅₀/ml |
| wells | (10^{x}) |
| 1 | 0.74 |
| 2 | 0.83 |
| 3 | 0.92 |
| 4 | 1.00 |
| 5 | 1.09 |
| 6 | 1.17 |
| 7 | 1.26 |
| 8 | 1.35 |
| 9 | 1.44 |
| 10 | 1.52 |
| 11 | 1.61 |
| 12 | 1.70 |
| 13 | 1.79 |
| 14 | 1.87 |
| 15 | 1.96 |
| 16 | 2.05 |
| 17 | 2.14 |
| 18 | 2.22 |
| 19 | 2.31 |
| 20 | 2.40 |
| 21 | 2.49 |
| 22 | 2.57 |
| 23 | 2.66 |
| 24 | 2.75 |
| 25 | 2.83 |
| 26 | 2.92 |
| 27 | 3.01 |
| 28 | 3.10 |
| 29 | 3.18 |
| 30 | 3.27 |
| 31 | 3.36 |
| 32 | 3.45 |
| 33 | 3.53 |
| 34 | 3.62 |
| 35 | 3.71 |
| 36 | 3.80 |
| 37 | 3.88 |
| 38 | 3.97 |
| 39 | 4.06 |
| 40 | 4.15 |
| 41 | 4.23 |
| 42 | 4.32 |
| 43 | 4.41 |
| 44 | 4.49 |
| 45 | 4.58 |
| 46 | 4.67 |
| 47 | 4.76 |
| 48 | 4.84 |
| 49 | 4.93 |
| 50 | 5.02 |
| 51 | 5.11 |
| 52 | 5.19 |
| 53 | 5.28 |
| 54 | 5.37 |
| 55 | 5.46 |
| 56 | 5.54 |
| 57 | 5.63 |
| 58 | 5.72 |
| 59 | 5.81 |
| 60 | 5.89 |
| 61 | 5.98 |
| 62 | 6.07 |
| 63 | 6.15 |
| 64 | 6.24 |
| 65 | 6.33 |
| 66 | 6.42 |
| 67 | 6.50 |
| 68 | 6.59 |
| 69 | 6.68 |
| 70 | 6.77 |
| 71 | 6.85 |
| 72 | 6.94 |
| 73 | 7.03 |
| 74 | 7.12 |
| 75 | 7.20 |
| 76 | 7.29 |
| 77 | 7.38 |
| 78 | 7.47 |
| 79 | 7.55 |
| 80 | 7.64 |

^{a}To calculate virus titer, first multiply the total number of positive wells by 2 (the chart was designed to be used with replicates of 8), then look up the corresponding TCID₅₀/mL titer and add 0.7 (the formula requires the addition of a log dilution factor).

### Data Analysis

Neutralization activity is displayed by plotting the percent inhibition of p24 antigen production (after background values are subtracted from all datapoints) versus log₁₀ drug concentration. The percent inhibition is derived as follows [1−(p24 levels in the presence of drug/p24 levels in the absence of drug)]×100. IC₅₀ values are determined by fitting the inhibition data with a non-linear, four-parameter, variable slope equation (GraphPad Prism, ver. 4.02; GraphPad Software, San Diego, Calif.). Upper and lower inhibition values are constrained to 100% and 0%, respectively for curve fitting.

### Synergy Determinations

Cooperative interactions between PRO 140 and small-molecule and peptide-based inhibitors of CCR5, CD4, HIV-1 gp120, HIV-1 gp41, HIV-1 reverse transcriptase and HIV-1 protease (Table 8) are determined as described for Example 1. Cooperative inhibition effects of drug combinations are determined by the method of Chou and Talalay (1984). IC₅₀ values are generated for all combinations as described above. Combination Index (CI) and Dose Reduction (DR) values are calculated according to the following formulas:

$$C l = ( \frac{I {C}_{50} D comb 1}{I {C}_{50} D solo 1} ) + ( \frac{I {C}_{50} D comb 2}{I {C}_{50} D solo 2} ) + α ( \frac{( I {C}_{50} D comb 1 ) ( I {C}_{50} D comb 2 )}{( I {C}_{50} D solo 1 ) ( I {C}_{50} D solo 2 )} )$$

DR (for compound 1)=(IC₅₀ Dsolo1/IC₅₀ Dcomb1)

DR (for compound 2)=(IC₅₀ Dsolo2/IC₅₀ Dcomb2)

### Where:

“IC₅₀ Dcomb1”=IC₅₀ of drug 1 in combination with drug 2;
“IC₅₀ Dsolo1”=IC₅₀ of drug 1 when tested alone;
“IC₅₀ Dcomb2”=IC₅₀ of drug 2 in combination with drug 1;
“IC₅₀ Dsolo2”=IC₅₀ of drug 2 when tested alone;
α=0 if the effects of the two drugs are mutually exclusive; and
α=1 if the effects of the two drugs are mutually nonexclusive.

Combinations with CI<1 are determined to be synergistic, whereas combinations with CI>1 are determined to be antagonistic. Additivity is reflected in combinations for which CI=1.

### Discussion

PRO 140 is a CCR5-specific mAb being developed for HIV-1 therapy. It is a humanized IgG4,κ version (see PCT International Publication No. WO 03/072766, published Sep. 4, 2003) of the murine antibody, PA14 (Olson et al., 1999; PCT International Publication No. WO 00/35409, published Jun. 20, 2000), which binds to the CCR5 receptor on the surface of a cell and inhibits CCR5-mediated fusion of HIV-1 to the cell. The studies described herein concern the testing of the antiviral activity of PRO 140 in combination with small-molecule and peptide inhibitors of HIV-1 infection. Data generated from this testing were analyzed for potential cooperative effects on inhibition of HIV-1 infection.

In one series of experiments, inhibition of HIV-1 infection was assayed using a fluorescence resonance energy transfer (RET) assay, which measures the fusion of effector cells (HeLa-Env_{JRFL}) expressing recombinant HIV-1 strain JRFL envelope glycoproteins (Env) to target cells (CEM NKR-CCR5) expressing CD4 and CCR5 (Litwin et al., 1996). In this assay, effector cells are labeled with the F18 dye and target cells with the R18 dye. HIV-1 Env-mediated fusion of effector and target cells results in the placement of these two dyes within close proximity in the cell membrane. When F18 is excited at its optimum wavelength (450 nm), it emits light at a wavelength (530 nm) that will excite R18 when the two dyes are co-localized in the same membrane, resulting in R18-specific emission at 590 nm. Drug susceptibility is measured by adding serial concentrations of drugs to target cells prior to addition of effector cells. Inhibition of HIV-1 Env-mediated fusion is reflected in a reduction in fluorescence emission due to R18 in a dose-dependent manner, providing a quantitative measure of drug activity.

Initial experiments measuring inhibition of HIV-1 Env-mediated fusion were conducted in order to demonstrate the robustness of the assay system for quantifying cooperative interactions. In these experiments, PRO 140 was run in combination with itself, a combination that should result in combination index (CI) values indicative of additive interactions. Using the methodology of Chou and Talalay (1984), CI values of <1.0, =1.0 and >1.0 are taken to indicate synergistic, additive and antagonistic interactions, respectively. Indeed, PRO 140 run in combination with itself returned a CI value of 0.97±0.08 (n=9; Table 7), indicating that the assay system accurately represented this interaction.

Synergy experiments were then conducted between PRO 140 and 3 small-molecule (SCH-D, TAK-779, UK427857), one peptide (RANTES) and one mAb (2D7) antagonist of CCR5. In addition, cooperative interactions were measured between PRO 140 and T-20 (peptide-based inhibitor of gp41), PRO 542 (protein-based inhibitor of gp120), BMS378806 (small molecule inhibitor of gp120) and Leu-3A (anti-CD4 mAb).

The results (see Table 7) revealed potent synergy between PRO 140 and all 3 small-molecule CCR5 antagonists as well as RANTES. CI values between PRO 140 and these CCR5 antagonists ranged from 0.36±0.10 to 0.59±0.08. Dose reduction values indicated that the compound in combination exerted about a 4-fold effect on PRO 140 activity, whereas the effect of PRO 140 on the compound in combination ranged from about 3- to about 16-fold (Table 7). Modest synergy to additivity was observed between PRO 140 and T-20, PRO 542, BMS-378806 and 2D7 (CI=0.84±0.16, 0.96±0.17, 1.21±0.21, and 0.93±0.04, respectively).

Small molecule antagonists of CCR5 run in combination (SCH-D and TAK-779) returned a mean CI value of 1.12±0.32, indicating a slightly additive interaction (Table 8). Conversely, the combination of the recombinant antibody-like fusion protein PRO 542 with the anti-CD4 mAb, Leu-3A, resulted in a mean CI value of 16.9±0.3, indicating potent antagonism between these two HIV-1 inhibitors (Table 8).

Varying the molar ratios of compounds demonstrated similar patterns of cooperativity. At both 5:1 and 1:5 molar ratios of PRO 140 to SCH-D, TAK-779, UK-427,857 and RANTES, potent synergistic inhibition of HIV-1-Env-mediated entry was observed (Table 9). This represents a broad range of inhibitor mass ratios, from a low of 0.15 to a high of 1,820. CI values between PRO 140 and CCR5 antagonists ranged from 0.52±0.20 to 0.84±0.14. More modest synergy to additivity was observed for combinations of PRO 140 with T-20, PRO 542 or BMS-378806. The results of these investigations identify clearly the potent synergistic activities of PRO 140 with CCR5 antagonists, as well as more modest synergy between PRO 140 and T-20 (see FIG. 4).

The HIV-1 inhibitory activity of the CCR5-specific murine mAb, 2D7, in combination with the small-molecule CCR5 antagonists and with RANTES, was also tested using the fluorescent RET assay. 2D7 was found to act synergistically with these CCR5 antagonists and with RANTES (Table 10). CI values between 2D7 and these CCR5 antagonists ranged from 0.15±0.03 to 0.62±0.04. Dose reduction values indicated that the compound in combination exerted about a 2- to 3-fold effect on 2D7 activity, except for TAK-779 which had an approximately 17-fold effect on 2D7 activity. The effect of 2D7 on the compound in combination ranged from about 2- to about 12-fold (Table 10). As observed previously, PRO 140 and 2D7 in combination were essentially additive or modestly synergistic (CI=0.93±0.04).

These results indicate that synergistic inhibition of HIV-1 Env-mediated cell-cell fusion is observed between multiple mAbs and small molecules that bind to CCR5. This property may be broadly applicable to mAbs that target CCR5, including, for example, the mAb CCR5 mAb004 that has been shown to bind to and antagonize CCR5 and block HIV-1 entry in a cell-cell fusion assay (Roschke et al., 2004). A large and growing number of small molecules have been identified as CCR5 antagonists (see Table 12). Certain of these small molecule CCR5 antagonists may also produce synergistic inhibition of HIV-1 Env-mediated fusion in combination with PRO 140 and other anti-CCR5 mAbs.

An alternative approach for examining synergistic interactions utilizes a virus-cell fusion assay as described previously (Nagashima et al., 2001; Trkola et al., 1998). In this assay an HIV genomic vector (pNLluc⁺Env⁻) containing a luciferase reporter gene is pseudotyped with Env from HIV-1_{JRFL}. Recombinant pseudotyped virus particles are used to infect U87 cells expressing CD4 and CCR5 (U87-CD4-CCR5). Production of luciferase in target cells is dependent on virus entry and the completion of one round of virus replication. Drug susceptibility is measured by adding serial concentrations of drugs to target cells prior to addition of pseudotyped virus particles. Inhibition of virus entry is reflected in a reduction in luciferase activity in a dose-dependent manner, providing a quantitative measure of drug susceptibility. Since the HIV genomic vector requires expression of functional HIV-1 reverse transcriptase (RT) to drive luciferase expression, this pseudovirus assay is also sensitive to inhibition by nucleotide/nucleoside reverse transcriptase inhibitors (NRTIs) and non-nucleoside reverse transcriptase inhibitors (NNRTIs). As such, the HIV-1pp assay is suitable for examining cooperative interactions between PRO 140 and small-molecule, peptide and protein inhibitors of CCR5, CD4, HIV-1 gp120, HIV-1 gp41 and HIV-1 reverse transcriptase.

TABLE 12
Small-Molecule CCR5 antagonists

| Small-Molecule CCR5 antagonist | Reference |
|---|---|
| 1,3,4-trisubstituted pyrrolidines | Kim et al., 2005 |
| Modified 4-piperidinyl-2-phenyl-1-(phenylsulfonylamino)- | Shah et al., 2005 |
| butanes | |
| Anibamine.TFA, Ophiobolin C, and 19,20-epoxycytochalasin Q | Jayasuriya et al., 2004 |
| 5-(piperidin-1-yl)-3-phenyl-pentylsulfones | Shankaran et al., 2004a |
| 4-(heteroarylpiperdin-1-yl-methyl)-pyrrolidin-1-yl-acetic acid | Shankaran et al., 2004b |
| antagonists | |
| Agents containing 4-(pyrazolyl)piperidine side chains | Shu et al., 2004 |
| Agents containing 4-(pyrazolyl)piperidine side chains. | Shen et al., 2004a; 2004b |
| 3-(pyrrolidin-1-yl)propionic acid analogues | Lynch et al., 2003c |
| [2-(R)-[N-methyl-N-(1-(R)-3-(S)-((4-(3-benzyl-1-ethyl-(1H)- | Kumar et al., 2003 |
| pyrazol-5-yl)piperidin-1-yl)methyl)-4-(S)-(3- | |
| fluorophenyl)cyclopent-1-yl)amino]-3-methylbutanoic acid | |
| (MRK-1)] | |
| 1,3,4 trisubstituted pyrrolidines bearing 4-aminoheterocycle | Willoughby et al., 2003; Lynch |
| substituted piperidine side chains | et al., 2003a; Lynch et al., |
| | 2003b; Hale et al., 2002 |
| Bicyclic isoxazolidines | Lynch et al., 2002 |
| Combinatorial synthesis of CCR5 antagonists | Willoughby et al., 2001 |
| Heterocycle-containing compounds | Kim et al., 2001b |
| Antagonists containing hydantoins | Kim et al., 2001a |
| 1,3,4 trisubstituted pyrrolidines | Hale et al., 2001 |
| 1-[N-(methyl)-N-(phenylsulfonyl)amino]-2-(phenyl)-4-(4-(N- | Finke et al., 2001 |
| (alkyl)-N-(benzyloxycarbonyl) | |
| amino)piperidin-1-yl)butanes | |
| Compounds from the plant Lippia alva | Hedge et al., 2004 |
| Piperazine-based CCR5 antagonists | Tagat et al., 2004 |
| Oximino-piperidino-piperidine-based CCR5 antagonists | Palani et al., 2003b |
| Rotamers of SCH 351125 | Palani et al., 2003a |
| Piperazine-based symmetrical heteroaryl carboxamides | McCombie et al., 2003 |
| Oximino-piperidino-piperidine | Palani et al., 2002 |
| amides | |
| Sch-351125 and Sch-350634 | Este, 2002 |
| SCH-C | Strizki et al., 2001 |
| 1-[(2,4-dimethyl-3-pyridinyl)carbonyl]-4-methyl-4-[3(S)- | Tagat et al., 2001a |
| methyl-4-[1(S)-[4-(trifluoromethyl)phenyl]ethyl]-1- | |
| piperazinyl]-piperidine N1-oxide (Sch-350634) | |
| 4-[(Z)-(4-bromophenyl)- | Palani et al., 2001 |
| (ethoxyimino)methyl]-1′-[(2,4-dimethyl-3- | |
| pyridinyl)carbonyl]-4′-methyl-1,4′-bipiperidine N-oxide (SCH | |
| 351125) | |
| 2(S)-methyl piperazines | Tagat et al., 2001b |
| Piperidine-4-carboxamide derivatives | Imamura et al., 2005 |
| 1-benzazepine derivatives containing a sulfoxide moiety | Seto et al., 2005 |
| anilide derivatives containing a pyridine N-oxide moiety | Seto et al., 2004a |
| 1-benzothiepine 1,1-dioxide and 1-benzazepine derivatives | Seto et al., 2004b |
| containing a tertiary amine moiety | |
| N-[3-(4-benzylpiperidin-1-yl)propyl]-N,N′-diphenylureas | Imamura et al., 2004a |
| 5-oxopyrrolidine-3-carboxamide derivatives | Imamura et al., 2004b |
| Anilide derivatives with a quaternary ammonium moiety | Shiraishi et al., 2000 |
| AK602/ONO4128/GW873140 | Nakata et al., 2005 |
| Spirodiketopiperazine derivatives | Maeda et al., 2001; Maeda et |
| | al., 2004 |
| Selective CCR5 antagonists | Thoma et al., 2004 |

A third approach for examining antiviral synergy utilizes a whole virus assay. Cooperativity between all classes of inhibitor molecules can be examined in this assay format.

In both the virus-cell fusion luciferase assay and the whole virus assay, IC₅₀ values are generated for all combinations as described herein for the RET assay. Cooperative inhibition effects of drug combinations are determined by the method of Chou and Talalay (1984).

PRO 140 broadly and potently inhibited CCR5-mediated HIV-1 entry without CCR5 antagonism or other immunologic side effects in preclinical testing. More recently, PRO 140 has demonstrated favorable tolerability, PK and immunologic profiles in preliminary results from an ongoing Phase 1a study in healthy volunteers. Thus, in many respects, PRO 140 offers a novel and attractive product profile for anti-HIV-1 therapy. Moreover, the activities of anti-CCR5 mAbs are fundamentally distinct from, but complementary to, those of small-molecule CCR5 antagonists (see Table 2).

It might have been expected that combinations of anti-CCR5 mAbs and non-antibody CCR5 antagonists would produce additive effects in inhibiting fusion of HIV-1 to CD4⁺CCR5⁺ target cells since both classes of agents bind to the same target molecule. Surprisingly, however, the data presented herein reveal that anti-CCR5 mAbs, exemplified by PRO 140 and 2D7, exhibited potent and reproducible synergy with non-antibody CCR5 antagonists, exemplified by SCH-D, TAK-779, UK-427,857 and RANTES, in inhibiting HIV-1 Env-mediated cell-cell fusion. Synergies routinely translated into 4- to 10-fold dose reductions, suggesting significant improvement in inhibitory potency for the drug combinations. In contrast, purely additive effects were observed for combinations of non-antibody CCR5 antagonists. These findings likely reflect the different patterns of CCR5 recognition of these molecules: whereas small-molecule CCR5 antagonists bind a common hydrophobic pocket within the transmembrane domains of CCR5, PRO 140 recognizes a hydrophilic, extracellular epitope of CCR5. Overall, the data support the use of PRO 140 in combination with non-antibody HIV-1 entry inhibitors and suggest that PRO 140 represents a distinct subclass of CCR5 inhibitor.

Moreover, the available data suggest that the observed synergy may also be exhibited by combinations involving anti-CCR5 mAbs other than PRO 140, including, but not limited to, mAb CCR5 mAb004 (Roschke et al., 2004), as well as non-antibody CCR5 antagonists other than SCH-D, TAK-779, UK-427,857 and RANTES. Thus, these antibodies likely produce synergistic effects in combination with GW873140 (Lalezari et al., 2004), TAK-652 (Baba et al., 2005), and at least certain of the small-molecule CCR5 antagonists listed in Table 12. Accordingly, combination therapy comprising administration of anti-CCR5 mAbs and non-antibody CCR5 antagonists may offer powerfully effective, new approaches to preventing and treating HIV-1 infection. It is expected that such therapy will result in more potent and more durable ant-HIV-1 treatments. Additionally, the synergistic effects described herein may enable a reduction in dosages of drugs administered to a subject as well as a reduction in dosing frequency.

### Example 4

### Loading and Maintenance Dose Regimens

The loading regimen, which can, for example, be more dose-intensive than the maintenance regimen, can, for example, have the following characteristics:

Number of doses: 1 or more (up to about 5 doses).

Dose level: About 25%, 50%, 75%, 100%, 150% or 200% greater than the maintenance dose regimen.

Dose frequency: About 1.5×, 2×, 3× or 4× more frequently than the maintenance dose regimen.

As an example, if the maintenance dose regimen is 2 mg/kg every two weeks, the loading dose regimen could comprise weekly 2 mg/kg doses. Alternatively, the loading dose regimen could comprise a single 4 mg/kg dose or multiple 4 mg/kg doses at weekly or biweekly intervals.

The loading dose regimen can be designed, for example, so as to accelerate the achievement of a pharmacokinetic steady state in the subject, as defined by uniform peak and trough blood concentrations of drug between doses. A preferred loading dose regimen can be determined by routine experimentation wherein the drug is administered to the subject by differing loading and maintenance regimens, and blood levels of drug are measured.

Also, in another embodiment, PRO 140 is administered according to a fixed-dose regimen such as, for example, 75 mg, 150 mg, 300 mg and 600 mg per administration.

### Part III

### Materials And Methods

### Inhibitors

PRO 140 was expressed in mammalian cells and purified by protein A, ion exchange and hydroxyapatite chromatographies. UK427,857 (Dorr et al. 2005), SCH-D (Tagat et al. 2004), TAK-779 (Baba et al. 1999), enfuvirtide (T-20 (Wild et al. 1992); BMS-378806 (Lin et al. 2003)) and PRO 542 (CD4-IgG2, (Allaway et al. 1995)) were prepared according to published methods. Zidovudine (azidothymidine, AZT), RANTES, the CCR5 mAb 2D7 and the CD4 mAb Leu-3A were purchased from Sigma Chemicals (St. Louis, Mo.), R&D Systems (Minneapolis, Minn.), Pharmingen (San Diego, Calif.), and Becton Dickinson (Franklin Lakes, N.J.), respectively. UK427,857 and SCH-D were radiolabeled with tritium by GE Healthcare (Piscataway, N.J.), and PRO 140 was conjugated to phycoerythrin (PE) by Southern Biotech, Inc. (Birmingham, Ala.).

### HIV-1 Membrane Fusion Assay

HIV-1 envelope-mediated membrane fusion was examined using a fluorescence resonance energy transfer (RET) assay (Litwin et al. 1996) with modifications. Briefly, HeLa cells that stably express HIV-1_{JRFL} gp120/gp41 (Litwin et al. 1996) and CEM.NKR-CCR5 cells (NIH AIDS Research and Reference Reagent Program, (Spenlehauer et al. 2001; Trkola et al. 1999)) were labeled separately overnight with fluorescein octadecyl ester (F18; Molecular Probes, Eugene, Oreg.) and rhodamine octadecyl ester (R18; Molecular Probes), respectively. Cells were washed in phosphate-buffered saline containing 15% fetal bovine serum (PBSF) and co-seeded at 15,000 cells/well into a 384-well plate. Inhibitors were added, and the plates were incubated in PBSF plus 0.5% dimethlysulfoxide (DMSO) for 4 h at 37° C. prior to measurement of RET using a Victor² plate reader (Perkin-Elmer, Boston, Mass.) as previously described (Litwin et al. 1996). The CD4 mAb Leu3a was used as a control inhibitor, and percent inhibition was calculated as: (RET in the absence of inhibitor−RET in the presence of inhibitor)/(RET in the absence of inhibitor−RET in the presence of Leu3a)×100.

### HIV-1 Pseudovirus Assay

A self-inactivating (SIN) vector was derived from the pNL4-3ΔEnv-luciferase vector (Dragic et al. 1996) by deleting 507 basepairs in the U3 region of the 3′ long terminal repeat (LTR) so as to remove the TATA box and transcription factor binding sites. The human cytomegalovirus promoter was inserted upstream of the luciferase (luc) gene to enable expression of luciferase following integration.

Reporter viruses pseudotyped with HIV-1_{JR-FL} or HIV-1_{SF162} envelopes were generated by cotransfection of 293T cells with the SIN vector and the appropriate pcDNA env-expressing vector as previously described (Dragic et al. 1996). U87-CD4-CCR5 cells (8,000/well; NIH AIDS Research and Reference Reagent Program) were infected with 125-375 pg of HIV-1 pseudoviruses in 384-well plates in the presence or absence of inhibitor(s). Cultures were incubated for 72 h at 37° C. in DMEM containing 10% fetal bovine serum, 1 mg/mL puromycin, 0.3 mg/mL geneticin, antibiotics, and 0.5% DMSO. Luciferase activity (relative light units or RLU) was measured using BrightGlo reagent (Promega, Madison, Wis.) according to the manufacturer's instructions. Percent inhibition was calculated as: (1−RLU in the presence of inhibitor/RLU in the absence of inhibitor)×100. IC50 and IC90 were used to denote the respective concentrations required for 50% and 90% inhibition of HIV-1.

### Synergy Determinations

Experimental design and data analysis were based on the combination index (CI) method (Chou et al. 1991; Chou et al. 1984). Compounds were tested individually and in combination at a fixed molar ratio over a range of serial dilutions. Entry inhibitors were combined in equimolar amounts, whereas a 1:10 molar ratio was used for PRO 140 in combination with azidothymidine and nevirapine. Dose-response curves were fit using a four-parameter sigmoidal equation with upper and lower inhibition values constrained to 100% and 0%, respectively, in order to calculate concentrations required for 50% (IC₅₀) and 90% (IC90) inhibition (GraphPad Prism, GraphPad Software, San Diego, Calif.). CI values for 50% (CI50) and 90% (CI90) inhibition were calculated as previously described (Chou et al. 1991; Chou et al. 1984). The mutually exclusive CI formula was used for combinations of CCR5 inhibitors, while the mutually non-exclusive formula was utilized for combinations of inhibitors to distinct targets (Chou et al. 1991). Each test was conducted 4-12 times. Synergy, additivity and antagonism are indicated by CI<1, CI=1 and CI>1, respectively.

### Competition Binding Assays

To examine inhibition of PRO 140 binding, CEM.NKR-CCR5 cells were suspended in phosphate-buffered saline with 0.1% sodium azide (PBSA) and incubated with varying concentrations of unlabeled CCR5 antagonists at ambient temperature for 30 minutes. Azide was added to block CCR5 internalization during the assay. Cells were washed in PBSA and incubated with 5 nM PRO 140-PE for an additional 30 minutes prior to washing and analysis by flow cytometry using a FACSCalibur instrument (Becton Dickinson). The extent of PRO 140-PE binding was measured in terms of both the mean fluorescence intensity (MFI) and the percent of cells gated for positive staining.

To examine inhibition of UK-427,857 binding, CEM.NKR-CCR5 cells were pre-incubated with unlabeled CCR5 inhibitors as described above prior to addition of 2 nM ³H-UK-427,857 for an additional 30 minutes. The cells were washed in PBSA and lysed with 0.5N HCl prior to scintillation counting using a Wallac1410 instrument. An additional study reversed the order of addition in order to examine the stability of UK-427,857 binding over the course of the assay. Cells were pre-incubated with 2 nM ³H-UK-427,857 for 30 min prior to washing, addition of unlabeled inhibitors, and processing as described above. EC50 and EC90 were used to denote the concentrations of unlabeled compound required to inhibit binding of labeled compound by 50% and 90%, respectively.

### Statistical Analyses

Two-tailed t-tests were used to test mean CI50 and CI90 values for the null hypothesis H₀: CI=1 (additivity) using GraphPad Prism software. P values were corrected for multiple comparisons from α=0.05 according to the Bonferroni method (Cudeck and O'Dell 1994), excluding the PRO 140/PRO 140 mock combination that was included as an assay control. In the Bonferroni correction, P=α/n, where n is the number of comparisons. Twenty-two synergy comparisons (11 compounds×2 CI values) were made based on data generated in the membrane fusion assay, resulting in a corrected P value of 0.0023. In the pseudovirus assay, 32 synergy comparisons (8 compounds×2 viruses×2 CI values) resulted in a corrected P value of 0.0016.

### Results

### Inhibition of HIV-1 Membrane Fusion

PRO 140 and UK-427,857 were used individually and together to inhibit HIV-1_{JR-FL} envelope-mediated membrane fusion in the RET cell-cell fusion assay, and representative dose-response curves for the individual agents and combination are illustrated in FIG. 11A. Although both PRO 140 and UK-427,857 individually blocked HIV-1 fusion at low nanomolar potency, the combination was markedly more potent. In this assay, 50% inhibition was obtained using 2.9 nM PRO 140 alone, 5.0 nM UK-427,857 used alone, or 2.1 nM of the combination (1.05 nM PRO 140 plus 1.05 nM UK-427,857). This supra-additive effect is indicative of antiviral synergy between the two agents. In contrast, the combination of SCH-D and UK-427,857 was no more potent than individual agents (FIG. 11B). In this example, the dose-response curves for the individual inhibitors and the combination were overlapping, with 50% inhibition requiring 9.7 nM UK427,857, 5.5 nM SCH-D and 6.1 nM of the combination. The data suggest purely additive effects for these inhibitors.

These studies were extended to additional CCR5 (TAK-779, RANTES and 2D7), gp120 (BMS-378806 and PRO 542) and gp41 (enfuvirtide) inhibitors, and were repeated four or more times for each condition. CI50 and CI90 values were calculated for each condition and averaged across the independent assays. Cooperativity was assessed using t-tests to determine if the CI50 and CI90 values were significantly different from one. As a test of these methods, a PRO 140/PRO 140 mock combination was examined by adding PRO 140 to the assay wells in two separate additions. CI50 and CI90 values for the PRO 140/PRO 140 combination were 0.96 and 0.97, respectively (Table 13); therefore, purely additive effects were observed for this mock combination, as expected.

TABLE 13
CI values for inhibition of HIV-1_{JR-FL} envelope-mediated membrane fusion^{a}

| 1^{st} | | | IC90, | | | | | |
|---|---|---|---|---|---|---|---|---|
| Inhibitor | Target | IC50, nM | nM | 2^{nd} Inhibitor | CI50 | P value | CI90 | P value |
| PRO 140 | CCR5 | 2.5 | 8.6 | PRO 140 | 0.97 ± 0.07 | 0.13 | 0.96 ± 0.14 | 0.37 |
| UK-427,857 | CCR5 | 5.3 | 27 | PRO 140 | [symbol] | [symbol] | [symbol] | [symbol] |
| SCH-D | CCR5 | 3.2 | 16 | PRO 140 | [symbol] | [symbol] | [symbol] | [symbol] |
| TAK-779 | CCR5 | 11 | >200 | PRO 140 | [symbol] | [symbol] | N/A | N/A |
| RANTES | CCR5 | 2.4 | 38 | PRO 140 | [symbol] | [symbol] | [symbol] | [symbol] |
| RANTES | CCR5 | 2.4 | 38 | UK-427,857 | [symbol] | [symbol] | [symbol] | [symbol] |
| SCH-D | CCR5 | 3.2 | 16 | UK-427,857 | 0.86 ± 0.03 | 0.016 | 0.75 ± 0.02 | 0.0033 |
| SCH-D | CCR5 | 3.2 | 16 | TAK-779 | 1.3 ± 0.18 | 0.12 | N/A | N/A |
| 2D7 | CCR5 | 3.7 | 58 | PRO 140 | 1.0 ± 0.14 | 0.61 | 1.9 ± 0.61 | 0.024 |
| enfuvirtide | gp41 | 8.6 | 66 | PRO 140 | 0.84 ± 0.16 | 0.040 | 0.89 ± 0.20 | 0.19 |
| PRO 542 | gp120 | 8.9 | 91 | PRO 140 | 0.96 ± 0.17 | 0.56 | 0.94 ± 0.19 | 0.45 |
| BMS-378806 | gp120 | 5.2 | 20 | PRO 140 | [symbol] | [symbol] | 1.1 ± 0.22 | 0.19 |

^{a}Statistically significant results (P < 0.0023 after application of the Bonferroni correction for multiple comparisons) are indicated in italicized bold text.
IC50 and IC90 denote values for the 1^{st} inhibitor.
N/A = not applicable;
TAK-779 did not consistently achieve 90% inhibition in the assay.
CI values represent the means and standard deviations of 4-12 independent assay

Potent synergy was observed for PRO 140 in combination with each of three small-molecule CCR5 antagonists (UK427,857, SCH-D and TAK-779), and the findings were statistically significant even when the data were corrected for multiple comparisons via the Bonferroni method (Table 13). CI values ranged from 0.36 to 0.61, and these synergies translated into dose reductions ranging from 3- to 8-fold across the different conditions. Synergies were greater at 90% inhibition than at 50% inhibition. Synergy between PRO 140 and small-molecule CCR5 antagonists was robust in that it was observed at both the 50% and 90% inhibition levels in every instance. The exception was TAK-779, which did not mediate 90% inhibition when used individually, and therefore a CI90 was not determined. Similarly potent synergy was observed when RANTES was used in combination with either PRO 140 or UK-427,857 (Table 13). Additional tests examined combinations of two small-molecule CCR5 antagonists (SCH-D/UK-427,857 and SCH-D/TAK-779) or two CCR5 mAbs (PRO 140/2D7). No significant synergy was observed for these combinations, although the SCH-D/UK-427,857 CI90 values trended towards significance. The findings are consistent with prior observations of overlapping binding sites for PRO 140 and 2D7 (Olson et al. 1999) and for SCH-D and TAK-779 (Seibert et al. 2006). PRO 140 was also tested in combination with the gp41 fusion inhibitor enfuvirtide and with the gp120 attachment inhibitors PRO 542 and BMS-378806 (Table 13). CI values ranged from 0.84 to 1.28, and none of these combinations demonstrated synergy that met the criteria for statistical significance. For the PRO 140/BMS-378806 combination, modest antagonism was observed at 50% but not 90% inhibition. The biological significance of this result is unclear.

### Inhibition of HIV-1 Pseudoviruses

Single-cycle HIV-1 reporter viruses were used to examine whether the synergistic effects were limited to cell-cell fusion or whether they extended to other modes of HIV-1 entry. Signals in this assay require both viral entry and reverse transcription, so that both NRTI and NNRTI may be included in the analyses. Each combination was tested against reporter viruses pseudotyped with envelopes from HIV-1_{JR-FL} and HIV-1_{SF162} in at least 4 independent assays per virus. A PRO 140/PRO 140 mock combination was again included as an assay control, and demonstrated additive effects against both HIV-1_{JR-FL} and HIV-1_{SF162} pseudoviruses, as expected (Table 14).

PRO 140 potently synergized with both UK-427,857 and SCH-D in blocking virus-cell fusion, and the results met the criteria for statistical significance. Comparable levels of synergy were observed against both HIV-1_{JR-FL} and HIV-1_{SF162} pseudoviruses at 50% and 90% inhibition (Table 14), with CI values ranging from 0.18 to 0.64. These synergies translated into dose reductions ranging to 14-fold. These results are in good agreement with those obtained in the cell-cell fusion assay (Table 13). Neither TAK-779 nor RANTES mediated consistent, high-level inhibition of HIV-1 pseudovirus entry, and therefore these compounds were not included in this analysis (data not shown).

TABLE 14
CI values for inhibition of HIV-1 reporter viruses pseudotyped with envelopes from
HIV-1_{JR-FL} and HIV-1_{SF162}^{a}.

| | | HIV-1 | IC50, | IC90, | | | | | |
|---|---|---|---|---|---|---|---|---|---|
| 1^{st} Inhibitor | Target | Envelope | nM | nM | 2^{nd} Inhibitor | CI50 | P value | CI90 | P value |
| PRO 140 | CCR5 | JRFL | 2.2 | 28 | PRO 140 | 1.2 ± 0.32 | 0.16 | 0.90 ± 0.15 | 0.047 |
| | | SF162 | 1.3 | 20 | PRO 140 | 1.0 ± 0.27 | 1.0 | 0.86 ± 0.33 | 0.21 |
| SCH-D | CCR5 | JRFL | 2.4 | 44 | PRO 140 | [symbol] | [symbol] | [symbol] | [symbol] |
| | | SF162 | 0.34 | 14 | PRO 140 | [symbol] | [symbol] | [symbol] | [symbol] |
| UK-427,857 | CCR5 | JRFL | 7.4 | 46 | PRO 140 | [symbol] | [symbol] | [symbol] | [symbol] |
| | | SF162 | 0.87 | 13 | PRO 140 | [symbol] | [symbol] | [symbol] | [symbol] |
| UK-427,857 | CCR5 | JRFL | 7.4 | 46 | SCH-D | 0.71 ± 0.11 | 0.16 | 1.2 ± 0.15 | 0.32 |
| | | SF162 | 0.87 | 13 | SCH-D | 0.87 ± 0.06 | 0.19 | 0.86 ± 0.28 | 0.61 |
| 2D7 | CCR5 | JRFL | 8.8 | >200 | PRO 140 | 1.5 ± 0.25 | 0.024 | N/A | N/A |
| | | SF162 | 2.2 | 74 | PRO 140 | 1.1 ± 0.47 | 0.61 | 1.0 ± 0.16 | 0.65 |
| PRO 542 | gp120 | JRFL | 0.19 | 2.9 | PRO 140 | 1.2 ± 0.32 | 0.22 | 1.0 ± 0.18 | 0.92 |
| | | SF162 | 0.36 | 7.1 | PRO 140 | 0.98 ± 0.28 | 0.84 | 0.64 ± 0.26 | 0.010 |
| BMS-378806 | gp120 | JRFL | 1.2 | 11 | PRO 140 | 1.2 ± 0.38 | 0.43 | 0.74 ± 0.23 | 0.059 |
| | | SF162 | 0.03 | 0.42 | PRO 140 | 1.1 ± 0.28 | 0.36 | 0.82 ± 0.21 | 0.068 |
| nevirapine | RT | JRFL | 30 | 310 | PRO 140 | 1.2 ± 0.38 | 0.36 | 0.73 ± 0.28 | 0.068 |
| | | SF162 | 42 | 280 | PRO 140 | 1.2 ± 0.34 | 0.30 | 0.63 ± 0.19 | 0.033 |
| zidovudine | RT | JRFL | 140 | 1900 | PRO 140 | 1.1 ± 0.38 | 0.37 | 0.85 ± 0.26 | 0.21 |
| | | SF162 | 86 | 2100 | PRO 140 | 0.99 ± 0.27 | 0.91 | 1.0 ± 0.38 | 1.0 |

^{a}Statistically significant results (P < 0.0016 after application of the Bonferroni correction for multiple comparisons) are indicated in italicized bold text.
IC50 and IC90 refer to values for the 1^{st} inhibitor.
N/A = not applicable;
2D7 did not consistently achieve 90% inhibition in the assay.
CI values represent the means and standard deviations of 4 or more independent assays

Additive effects were observed for both the UK-427,857/SCH-D and PRO 140/2D7 combinations (Table 14). Similarly, additivity was observed for PRO 140 used in combination with the gp120 inhibitors PRO 542 and BMS-378806. No antagonism was observed for the PRO 140/BMS-378806 combination against either virus. Overall, these findings are consistent with those seen for cell-cell fusion. Lastly, additive effects were observed for PRO 140 in combination with either zidovudine (NRTI) or nevirapine (NNRTI).

### Competition Binding Studies

As described above, additive antiviral effects were observed for inhibitors known (PRO 140 and 2D7) or inferred (UK-427,857 and SCH-D) to compete for CCR5 binding; however, little is known regarding the competitive binding of synergistic compounds (e.g., PRO 140/UK-427,857 and PRO 140/SCH-D). Since non-competitive binding provides a possible mechanism for synergy between CCR5 inhibitors, this issue was explored using labeled forms of UK-427,857 and PRO 140.

Flow cytometry was used to examine inhibition of PRO 140-PE binding to CEM.NRK.CCR5 cells by unlabeled PRO 140, UK-427,857 and SCH-D. PRO 140-PE binding was efficiently inhibited by unlabeled PRO 140, as expected. Complete inhibition was observed in terms of both MFI values (FIG. 12A) and the percent of cells gated for positive binding (FIG. 12B). The EC50 based on MFI data was 2.5 nM (FIG. 12A), and this value compares favorably with the antiviral IC50 of PRO 140 (Tables 13 and 14). Since percent cells gated is a readout for essentially complete inhibition of binding, an EC90 value was calculated as 17 nM, and this value is similar to the antiviral IC90 values observed for PRO 140 (Tables 13 and 14). 2D7 also completely inhibited binding of PRO 140-PE to CEM.NKR-CCR5. The CCR5 specificity of PRO 140-PE was also demonstrated by its inability to bind parental CEM.NKR cells.

In sharp contrast, modest levels of inhibition were observed for UK427,857 and SCH-D (FIG. 12). Micromolar concentrations of UK-427,857 and SCH-D reduced PRO 140-PE MFI values by 50% or less (FIG. 12A). More dramatically, UK-427,857 and SCH-D had little impact on the percent of cells gated for positive binding of PRO 140-PE (FIG. 12B). The findings suggest that UK427,857 and SCH-D partially reduce the number of PRO 140-PE molecules bound per cell; however, these compounds do not reduce the number of cells that bind measurable amounts of PRO 140-PE. Therefore, UK427,857 and SCH-D represent partial antagonists of PRO 140 binding, and this finding provides a mechanism for the antiviral synergy observed between PRO 140 and these small-molecule CCR5 antagonists.

Inhibition of ³H-UK427,857 binding by unlabeled UK427,857, SCH-D and PRO 140 was next examined. Binding of ³H-UK427,857 to CEM.NKR-CCR5 cells was efficiently inhibited by unlabeled UK427,857 (FIG. 13A). The EC50 for binding was 4.3 nM and is similar to the antiviral IC50 values observed for UK-427,857 (Tables 13 and 14).

SCH-D also blocked ³H-UK427,857 binding to background levels (FIG. 13A). However, there was no correlation between the compounds' antiviral potency and their potency in blocking ³H-UK427,857 binding. For example, whereas SCH-D demonstrated equal or slightly greater antiviral potency than UK427,857 (Tables 13 and 14), SCH-D was less potent in blocking ³H-UK-427,857 binding (EC50=17 nM, FIG. 13A). This result is consistent with minor differences in the CCR5 binding sites of these compounds.

Surprisingly, PRO 140 also blocked ³H-UK427,857 binding to background levels (FIG. 13A), and this result contrasts with the modest inhibition of PRO 140-PE binding by UK-427,857 (FIG. 12). PRO 140 inhibited ³H-UK-427,857 binding with an EC50 of 14 nM, which is 5-10 fold higher than the antiviral IC50 of PRO 140 (Tables 13 and 14).

A final experiment examined the stability of UK-427,857 binding to CEM.NKR-CCR5 cells under the conditions of the competition assay. For this, cells were pre-incubated with ³H-UK-427,857 and then the dissociation was examined in the presence of unlabeled UK427,857, SCH-D and PRO 140. As indicated in FIG. 13B, there was minimal dissociation of ³H-UK-427,857 over 30 min at ambient temperature, and UK427,857 wasn't displaced by either PRO 140 or SCH-D. Therefore, the inability of UK-427,857 to efficiently compete PRO 140 binding to CCR5 (FIG. 12) is not due to rapid dissociation of UK-427,857 from CCR5 during the course of the assay. Collectively, the data indicate that PRO 140 can bind CCR5 in the presence of pre-bound UK-427,857.

### Discussion

This study explores interactions between mAb and small-molecule CCR5 inhibitors and examines combinations of CCR5 drugs that currently are in development for HIV-1 therapy. Surprisingly, potent antiviral synergy between the CCR5 mAb PRO 140 and each of three structurally distinct small-molecule CCR5 antagonists was observed. Consistent, high-level synergy was observed across varying assay systems, viral isolates, target cells and inhibition levels. PRO 140 and small-molecule CCR5 antagonists were more potently synergistic when used together rather than in combination with inhibitors that block other stages of HIV-1 entry. In contrast, additive effects were observed for combinations of two small-molecule CCR5 antagonists. Competition binding studies revealed complex and non-reciprocal patterns of CCR5 binding by mAb and small-molecule CCR5 inhibitors, and suggest that the synergistic interactions occur at the level of receptor binding.

Robust synergy between mAb and small-molecule CCR5 inhibitors was observed in this study. Potent synergy was observed for both cell-cell and virus-cell fusion, and there was a good concordance of findings in these two well-established assay systems. Comparable levels of synergy were observed for PRO 140 in combination with each of 3 small-molecule CCR5 antagonists from unrelated chemical series. In addition, consistent synergy was observed for each of two well-characterized HIV-1 envelopes and two CCR5 target cells. Synergy increased with increasing levels of viral inhibition and translated into in vitro dose reductions of up to 14-fold. Viewed alternatively, this degree of synergy provides a corresponding increase in antiviral pressure at a given concentration of drugs, thereby improving viral suppression and potentially delaying the emergence of drug-resistant virus. This is supported by preliminary studies indicating the mAb and small-molecule CCR5 inhibitors possess complementary patterns of viral resistance (Kuhmann et al. 2004 and Marozsan et al. 2005). The present findings provide a rationale for clinical exploration of regimens that combine mAb and small-molecule CCR5 inhibitors.

Potent synergy was also observed for RANTES used in combination with either UK-427,857 or PRO 140. Endogenous levels of RANTES may afford some protection against HIV-1 disease progression during natural infection (Garzino-Demo et al. 1999; Lui et al. 1999), and therefore this finding of synergy has important and positive implications for CCR5-targeted therapies of HIV-1. Antiviral synergy between RANTES and PRO 140 is not surprising based on a prior observation that RANTES signaling is not blocked by antiviral concentrations of murine PRO 140 (PA14) (Olson et al. 1999).

Synergy between RANTES and UK-427,857 is less easily explained given that UK427,857 is a potent CCR5 antagonist. However, these findings are consistent with prior observations of synergy between the small-molecule CCR5 antagonist SCH-C and aminooxypentane-RANTES (AOP-RANTES) (Tremblay et al. 2002), a RANTES derivative that has been evaluated as a potential topical microbicide (Kawamura et al. 2000).

In contrast to the robust synergy observed between mAb and small-molecule CCR5 antagonists, additive effects were observed for combinations of small-molecule CCR5 antagonists. Lack of cooperativity is consistent with the view that these molecules compete for binding to a common pocket on CCR5 (Dragic et al. 2000; Nishikawa et al. 2005; Tsamis et al. 2003; Watson et al. 2005). The in vitro studies do not provide a basis for combining small-molecule CCR5 antagonists in the clinic based solely on inhibition of wild-type virus.

Similarly, potent synergy was not observed between PRO 140 and inhibitors of HIV-1 attachment (PRO 542 and BMS-378806), fusion (enfuvirtide), or reverse transcriptase (zidovudine and nevirapine), and these findings underscore the significance of the synergy observed for PRO 140 and small-molecule CCR5 antagonists. A number of prior studies have examined interactions between various small-molecule CCR5 antagonists (UK-427,857, SCH-C, TAK-220, TAK-652 and E913) and drugs from each of the existing HIV-1 treatment classes. Most (Tremblay et al. 2005 Antivir. Ther.; Tremblay et al. 2005 Antimicrob. Agents Chemother; Tremblay et al. 2002) but not all (Dorr et al. 2005; Maeda et al. 2001) studies have reported broad synergy between CCR5 inhibitors and the other HIV-1 treatment classes, and the divergent results may reflect differences in the compounds and methods used for antiviral testing as well as differences in the methods used for data analysis. When UK427,857 was tested against 20 licensed antiretroviral agents, additive effects were observed in all but three cases, where modest synergy was reported (Dorr et al. 2005). This result is consistent with the present findings for combinations of PRO 140 and HIV-1 inhibitors that do not target CCR5.

Without intending to be bound by theory, synergy between anti-HIV-1 drugs may stem from a variety of mechanisms. In mixed virus cultures, one compound may inhibit virus resistant to a second compound (Johnson et al. 1991), and NRTI NNRTI combinations may overcome specific RT-mediated resistance mechanisms (Basavapathruni et al. 2004; Borkow et al. 1999). Metabolic interactions between inhibitors may increase their effective intracellular drug concentrations (Molla et al. 2002), and synergistic entry inhibitors may disrupt interdependent steps in the entry cascade (Nagashima et al. 2001; Tremblay et al. 2000). The present study examined clonal viral envelopes rather than mixed populations, and the extracellular nature of the target argues against metabolic interactions. Multiple domains of gp120 contribute to CCR5 binding (Cormier et al. 2002), but it is unclear at present whether these interactions represent separate or discrete events during infection.

The present findings indicate that antiviral synergy between mAb and small-molecule CCR5 inhibitors may occur at the level of the receptor. As discussed above, mAbs and small molecules bind distinct loci on CCR5 (Dragic et al. 2000; Nishikawa et al. 2005; Tsamis et al. 2003; Olson et al. 1999; Watson et al. 2005). When pre-incubated with CCR5 cells in the present study, PRO 140 completely blocked subsequent binding of UK-427,857 to the receptor; although the PRO 140 concentrations were higher than those needed to block HIV-1 entry into the same cells. In contrast, pre-incubation of CCR5 cells with super-saturating concentrations of UK-427,857 or SCH-D reduced PRO 140 binding by 50% or less. As one possible explanation, PRO 140 could recognize CCR5 conformers that are not bound by UK-427,857 or SCH-D. Although cell-surface CCR5 exists in multiple conformations (Lee et al. 1999), it seems unlikely that the small-molecule antagonists could demonstrate potent antiviral activity while failing to bind a significant fraction of cell-surface CCR5. In this regard, it is important to note that a common cellular background (CEM.NKR-CCR5 cells) was used for competition binding and antiviral studies, and therefore the findings are not related to cell-specific differences in CCR5 expression.

Without intending to be bound by theory, another plausible explanation for the present findings is that PRO 140 is capable of forming a ternary complex with UK-427,857-bound CCR5, and this ternary complex provides an increased barrier to HIV-1 entry. Within the context of this model, PRO 140 may bind UK427,857-bound CCR5 somewhat less efficiently than free CCR5, as evidenced by the modest reduction in PRO 140 binding in the presence of UK-427,857.

The combination index method is widely used to assess drug-drug interactions. In this method, cooperativity often is defined on the basis of empirical CI values (e.g., <0.9 for synergy and >1.1 for antagonism) irrespective of inter-assay variability. Statistical analyses are performed infrequently, and even more rarely are adjustments made for multiple comparisons. In the absence of such analyses, there is increased potential to overestimate the number of synergistic combinations.

A rigorous and conservative approach to identifying synergistic effects was employed. CI values were tested for statistical significance against the null hypothesis of additivity (CI=1). In addition, these studies determined 20-30 different CI values per experiment (Tables 13 and 14), as is common in synergy studies. In order to reduce the potential for spurious positive results, the significance level was reduced using the Bonferroni correction. A mock combination was also evaluated as a test of these methods for antiviral testing and data analysis. It was therefore concluded that numerous apparent synergies (CI<0.9) could not be distinguished from inter-assay variation based on the available data. However, despite the rigorous nature of these methods, PRO 140 and small-molecule inhibitors demonstrated significant synergy under every test condition, lending credence to this finding. Combinations with CI values that trended towards significance in the present survey could be explored in future studies. For example, data for the PRO 140/enfuvirtide combination suggested modest synergy that trended towards significance; thus this combination may also be useful for treating HIV-1 infection.

A growing body of data indicates that mAb and small-molecule CCR5 antagonists represent distinct subclasses of CCR5 inhibitors, and a number of important parallels can be drawn between NRTI and NNRTI on the one hand and between mAb and small-molecule CCR5 antagonists on the other. In each instance, there are distinct binding loci for the inhibitors on the target protein (reverse transcriptase or CCR5). One set of inhibitors (NNRTI or small-molecule CCR5 antagonists) acts via allosteric mechanisms, while the other set (NRTI or CCR5 mAbs) acts as a competitive inhibitor. Like NRTI and NNRTI, mAb and small-molecule CCR5 inhibitors are synergistic and possess complementary patterns of viral resistance in vitro in preliminary testing (Kuhmann et al. 2004; Marozsan et al. 2005). NRTI and NNRTI represent important and distinct treatment classes even though they target the same protein, and mAb and small-molecule CCR5 inhibitors similarly may offer distinct HIV-1 treatment modalities.

### Part IV

### Materials and Methods

PRO 140 and small-molecule CCR5 antagonists were prepared and/or obtained as described herein above. The primary R5 HIV-1 isolates JR-FL and Case C 1/85 (CC1/85) were passaged weekly in vitro on peripheral blood mononuclear cells (PBMCC) in the presence or absence of progressively increasing concentrations of PRO 140 or SCH-D, and viral cultures were examined for susceptibility to these and other CCR5 inhibitors. For susceptibility testing, viruses were cultured in vitro on stimulated PBMC. In the presence and absence of serially diluted drug, and the extent of viral replication was determined by p24 ELISA.

### Results

For both JR-FL and CC1/85, drug-resistant variants were generated in the presence of PRO 140 and SCH-D. At passage 12, the escape mutants were approximately 10- to 100-fold less susceptible to the drug used for selection. In each case, the escape mutants continued to require CCR5 for replication on PBMC. Complementary patterns of resistance were observed: SCH-D escape mutants were efficiently inhibited by PRO 140 and PRO 140 escape mutants were efficiently inhibited by SCH-D.

### Discussion

PRO 140 escape mutants continue to require CCR5 for entry and remain susceptible to small-molecule CCR5 antagonists. In addition, PRO 140 is active against viruses resistant to small-molecule CCR5 antagonists. These findings indicate that PRO 140 and small-molecule CCR5 antagonists may represent distinct subclasses of CCR5 inhibitors.

### Part V

### Phase 1b Clinical Trial:

A Phase 1b, double-blind, randomized, single-dose, dose-cohort escalation study was conducted in which PRO 140 or placebo control was administered intravenously to adult (male and female) HIV-infected subjects. The efficacy data collected during the study were changes in viral load and CD4 counts over time. The safety data collected during the study were serious adverse events (SAEs)/adverse events (AEs) and changes in laboratory parameters (hematology, chemistry), physical exam, viral tropism and ECGs over time. The exploratory data collected included PK, immunogenicity (anti-PRO 140 antibody production), RANTES, and CCR5 lymphocyte coating over time.

### Clinical Trial Design and Study Results

This multi-center, double-blind, randomized, placebo-controlled phase 1b trial examined three single intravenous escalating doses of PRO 140: 0.5 mg/kg, 2.0 mg/kg and 5.0 mg/kg. The study was designed to assess the safety, tolerability, pharmacology and antiviral activity of PRO 140 through day 59 and was conducted at 10 sites in the United States. Thirty-nine HIV-infected individuals who had taken no anti-retroviral therapy within the preceding three months and who had plasma HIV RNA levels (viral loads) greater than or equal to 5,000 copies/mL were enrolled to receive PRO 140 monotherapy or placebo. The HIV-infected individuals in the study had a CD4+ count of >250 cells/μg. All patients were screened prior to the study for the presence of virus that utilizes only CCR5 as the entry coreceptor., i.e., CCR5-tropic virus. Of the 13 patients in each cohort, 10 patients received PRO 140 and three received placebo (10:3). A summary of the baseline characteristics of the patients in the study is presented in Table 15.

TABLE 15
Subject Baseline Characteristics

| | Placebo | 0.5 mg/kg | 2 mg/kg | 5 mg/kg | All Patients |
|---|---|---|---|---|---|
| Characteristic | (n = 9) | (n = 10) | (n = 10) | (n = 10) | (n = 39) |
| Age | 40.3 | 37.1 | 37.6 | 42.8 | 40.3 |
| median (range) | (23.8-50.2) | (24.1-53.2) | (23.2-51.5) | (22.9-61.1) | (22.9-61.1) |
| Gender (n) | 8/1 | 10/0 | 8/2 | 5/5 | 31/8 |
| male/female | | | | | |
| Race (n) | 4/5/0 | 4/4/2 | 4/6/0 | 5/4/1 | 17/19/3 |
| black/white/other | | | | | |
| Weight, kg | 81.4 | 81.0 | 81.7 | 73.4 | 80.9 |
| median (range) | (57.3-101.7) | (54.2-111.4) | (55.9-142.9) | (52.7-86.8) | (52.7-142.9) |
| CD4, cells/μl) | 439 | 492 | 438 | 535 | 484 |
| median (range) | (281-555) | (443-762) | (269-613) | (303-853) | (269-853) |
| Log₁₀ HIV RNA, | 4.44 | 4.45 | 4.44 | 4.37 | 4.43 |
| copies/mL | (3.98-5.61) | (3.79-5.54) | (3.89-4.94) | (3.81-5.36) | (3.79-5.61) |
| median (range) | | | | | |

The primary efficacy endpoint was the reduction in plasma HIV RNA level as measured by the Roche Amplicor™ Assay. The primary efficacy endpoint is the maximum change from baseline in viral load, defined as HIV-1 copies/ml, as measured by the Roche Amplicor™ Assay. In the Phase 1b study, the results were positive, dose-dependent, and highly statistically significant for the two highest doses tested. HIV-infected individuals who received 5.0 mg/kg of PRO 140 achieved an average maximum decrease of viral load of 1.83 log₁₀ (98.5%; P<0.0001), with individual reductions ranging up to 2.5 log₁₀ (99.7%) at the 2.0 mg/kg and 5.0 mg/kg dose levels. At nine days post-treatment, mean HIV RNA values nadired, and these same individuals achieved a mean viral load reduction of 1.70 log₁₀ (98%; P<0.0001). At this time, mean PRO 140 serum concentrations were 1.4 and 4.1 μg/ml in the 2.0 mg/kg and 5.0 mg/kg dose levels, respectively. In the 5.0 mg/kg cohort, mean viral load was suppressed by 1.0 log₁₀ (90%) within four days of dosing and persisted at or below the 1.0 log₁₀ level of reduction for two to three weeks in patients before returning to baseline at approximately 30 days. The response rate among the treatment groups (percentage of patients with a ≧1 log₁₀ decrease in HIV RNA at any time) increased with PRO 140 dose, reaching a maximum of 100% in the highest dose cohort (P<0.0001).

Mean log₁₀ HIV RNA changes of −0.13, −0.37, −1.04 (P=0.0001) and −1.70 (P=0.0001) were observed at Day 10 post-treatment for the placebo, 0.5 mg/kg, 2.0 mg/kg and 5.0 mg/kg groups, respectively, as shown in FIG. 22; and a >10-fold decrease in HIV RNA was observed in 0/9, 1/10, 6/10 (P=0.01) and 10/10 (P=0.0001) individuals in these respective treatment groups. All PRO 140-treated individuals had exclusively R5 virus at pre-dose (baseline) and at end of study; there was no change in viral susceptibility to PRO 140 during the course of the study. Antiviral effects were evaluated as functions of PRO 140 serum levels, CCR5 receptor occupancy and viral susceptibility.

The conclusions determined from this Phase 1b study are presented below:
PRO 140 at single doses of 2.0 mg/kg and 5.0 mg/kg were effective in reducing viral load.
A dose response and an ineffective dose were identified.
PRO 140 5 mg/kg efficacy was dramatic, as evidenced by:
   Maximum decrease of viral load at any time is −1.8 log, p<0.0001, as shown in FIGS. 19 and 21.
   Viral load decrease was statistically significant by day 5, remained statistically significant through day 15 (day 22 was −0.73, p=0.052), nominal p values, as shown in FIG. 19.
   Patients with >1 log decrease (anytime) 10/10 (100%), p<0.0001, as shown in FIG. 18.
   AUC viral load is significantly decreased (p=0.022), as shown in FIG. 18.
   Patients with <400 copies/mL (anytime) 4/10 (40%), p=0.087, as shown in FIG. 18.
   At 5 mg/kg, transient rise in and trend toward increased CD4+ lymphocytes (129 cells/mm³ (29%) average increase at Day 8), p=0.055, nominal p values as shown as shown in FIGS. 20A and 20B. Levels remained elevated for 3 weeks post-treatment.
Safety
   PRO 140 doses were well-tolerated in all study groups.
   No treatment related SAEs and no apparent dose-related AEs were observed.
   No dose-limiting toxicity or obvious pattern of toxicity was observed.
   No change in plasma RANTES (CCL5) chemokine levels was observed.
   No difference relative to placebo in HIV co-receptor tropism. No tropism shift (e.g., from CCR5 tropism to CXCR4 tropism) on treatment with PRO 140. All subjects screened for R5-only virus. Dual/mixed tropism result observed post-treatment in 1/9 placebo subjects (11%) and in 1/30 PRO 140 subjects (3%; 0.5 mg/kg group).
Immunogenicity
   One patient in the 5 mg/kg cohort had a positive titer (1:40) for anti-PRO 140 antibody at day 59.
   All other subjects in all cohorts tested negative.
Pharmacokinetics (PK)
   Serum concentrations of PRO 140 and anti-PRO 140 antibodies measured by ELISA. (FIG. 26).
   Concentrations [of PRO 140 in plasma] are 1% of Cmax by days 6-7.
   PK metrics assessed by non-compartmental analysis. Peak and total exposure increased proportionally or better with dose. Terminal half life approximately 4 d.
   Low titer anti-PRO 140 antibodies in one subject (in 5 mg/kg group). No obvious effect on PK or antiviral response.
Coating of CCR5 Lymphocytes
   Lymphocytes analyzed by flow cytometry ex vivo with fluorescently-labeled PRO 140 and non-competing CCR5 antibody.
   No depletion of CCR5+ lymphocytes.
   Obvious coating of CCR5 lymphocytes by PRO 140.
   Duration of coating maximal for 1-2 weeks and consistent with duration of antiviral effects. (FIG. 27).
Virus Susceptibility
   All viruses in PRO 140-treated subjects were susceptible to PRO 140 at baseline, with minimal variation. Mean rIC50=2.0 (range: 0.83-5.1). Mean Maximum Percent Inhibition (MPI)=99% (range: 93-100%).
   No change in susceptibility post-treatment. Less than 2-fold change in rIC50 for all subjects. Mean rIC50=2.1 (range: 0.98-4.71). Mean MPI=99% (range: 93-100%). (FIG. 25).
   No change from baseline to day 59 in either the IC50 or the Fold Change of PRO 140 or Fuzeon, as determined by the PhenoSense™ Assay.
Correlates of Efficacy
   PRO 140 dose was correlated with both the magnitude of HIV-1 RNA reduction (p=0.0001) and the duration of the response (p=0.0059).
   Correlation between HIV-1 RNA reduction and PRO 140 exposure (Eₘₐₓ analysis). (FIG. 28).
   HIV-1 RNA nadirs were independent of baseline HIV-1 RNA, baseline CD4+ cells and baseline CCR5+ cells. Accordingly, antiviral effects correlated with PRO 140 dose and exposure, but not with baseline HIV-1 RNA, CD4 cells, or CCR5 cells. (FIG. 29)
Preclinical studies support clinical use and feasibility of SC delivery
   Safety and tolerability of SC administration in a six-month, preclinical animal model study.
   Repeat SC dosing feasible.
   Weekly and q2weeks SC dosing expected to provide exposure similar to that of q2 weeks 5 mg/kg IV dose and may reduce differences in peak and trough concentrations.

The virological response rate was determined at the completion of the Phase 1b study, as shown in FIG. 23. Coreceptor tropism results (Trofile™, Monogram Biosciences), are shown in FIG. 24. For the placebo cohort, 1/9 subjects (11%, days 1, 8, 29 and 59) showed dual/mixed (D/M) tropism results. For the 0.5 mg/ml PRO 140 cohort, 1/30 subjects (3%, day 8 only) showed D/M tropism. Analysis of Env clones of pre-dose and D/M viruses can determine whether such low frequency of D/M tropism results from clones (CXCR4) that pre-existed PRO 140 treatment and which are phylogenetically highly related to on-treatment CXCR4-using virus. Where no CXCR4-using clones are identified in a pre-dose sample from a subject, the on-treatment CXCR4-using virus can be identified as being phylogenetically very distant from the CCR5 tropic baseline virus; in such a case, the emergence of a pre-dose or pre-treatment CXCR4-using virus, i.e., one that is present at baseline, but is not detected, may likely explain the D/M tropism in the subject, as opposed to a tropism switch.

### Part IV

PRO 140 and PA14, the murine precursor to PRO 140, were tested for the ability to inhibit replication of five CCR5-tropic (R5) HIV-2 isolates and two mixed/dual-tropic (R5X4) HIV-2 isolates in human peripheral blood mononuclear cells (PBMC). Two well-characterized R5 HIV-1 isolates were included for comparative purposes.

### Materials and Methods:

Viruses: All virus isolates were grown and titered exclusively in stimulated PBMC as described previously. HIV-1_{JR-FL} and HIV-1_{cc 1/85}, formerly known as HIV-1_{Case C 1/85} have been studied previously for susceptibility to PRO 140 and PA14. The HIV-2 isolates used in this study have been previously described (Owen, S. M., Ellenberger, D., Rayfield, M., Wiktor, S., Michel, P., Grieco, M. H., Gao, F., Hahn, B. H., and Lai, R. B. 1998. Genetically Divergent Strains of Human Immunodeficiency Virus Type 2 Use Multiple Coreceptors for Entry. J Virol 72:5425-5432) and were obtained from the NIH AIDS Research and Reference Reagent Program.

Inhibitors: PRO 140 (Lot #4027-COO) was produced in accordance with Good Manufacturing Practice. PA14 (Lot # DF-021204) was produced for reagent use in the Research & Development laboratories of Progenics Pharmaceuticals Inc.

Inhibition of HIV-1 and HIV-2 replication in PBMC cultures: PBMC were isolated from 34 healthy blood donors with Ficoll-Hypaque (all media from Cellgro, Herndon, Va.) and depleted of CD8+ cells using RosetteSep CD8 depletion cocktail (StemCell Technologies) according to the manufacturer's instructions. Equal numbers of cells from each donor were then separately stimulated with 5 ug phytohemagglutinin/ml (Sigma, St. Louis, Mo.) or with surface-immobilized anti-CD3 antibody OKT3 in RPMI 1640 medium containing 10% fetal calf serum, 100 Units of interleukin-2/ml (NIH AIDS Research and Reference Reagent Program), glutamine, and antibiotics (PBMC culture medium) as previously described. After 72 hours, equal numbers of PBMC stimulated by one of these two methods from each donor were combined for use in infection assays as follows. PBMC (1.4×10⁵) in 100 ul of PBMC culture medium were combined with 50 ul aliquots of serially diluted inhibitor for 1 h at 37° C. The virus inoculum was adjusted to 400 to 2,000 times the TCID₅₀ per ml in PBMC culture medium, and a 50-ul aliquot was added to each culture. The inhibitory doses refer to the concentrations of the agents present at this point in the culture, when virus, cells, and inhibitors were all present at their final concentrations. Reverse Transcriptase (RT) activity was measured on Day 7 post infection (pi) for HIV-1 and Day 10 pi for HIV-2 by the Reverse Transcriptase Assay, Colorimetric kit, #11 468 120 910, Roche (Switzerland) according to the manufacturer's instructions with the following exception: neither ultracentrifugation nor PEG precipitation of the culture supernatants was performed before viral lysis.

The inhibition data were analyzed using GraphPad Prism software (GraphPad Software, San Diego, Calif.). A 4-parameter logistic fit was used calculate the concentration of inhibitor that afforded a 50% and a 90% reduction in RT activity. These were designated the 50% and 90% inhibitory concentrations (EC₅₀ and EC₉₀). The % inhibitions where best-fit curve reached a top plateau, also as calculated by GraphPad Prism software, are reported as well as maximum percent inhibition (MPI). Inhibition assays were repeated 5 times, and these data were used to determine median EC₅₀, EC₉₀ and MPI values. Inhibition curves with a poor sigmoid fit (R²<0.7) were excluded from data analysis. In assays where <50% or <90% inhibition was obtained at the highest concentration of inhibitor tested (or >50% or >90% were obtained at the lowest concentration of inhibitor tested), the highest (or lowest) test concentration from that assay was used to calculate the median EC₅₀ or EC₉₀.

### Results:

PRO 140 and PA14 were tested against five R5 HIV-2 isolates. For comparison, PRO 140 and PA14 were also tested against two mixed/dual-tropic (R5X4) HIV-2 isolates, and two R5 HIV-1 reference isolates. The median concentrations of PRO 140 and PA14 that afforded 50% and 90% reductions in viral growth (EC₅₀ and EC₉₀, respectively) are listed below in Table 16. PRO 140 inhibited replication of each of the five R5 HIV-2 isolates in this study, and the susceptibility of HIV-2 was similar to that of the reference R5 HIV-1 isolates. PRO 140 and PA14 showed comparable activities in this study. Neither PRO 140 nor PA14 measurably affected replication of R5X4 HIV-2 isolates.

TABLE 16
Median PRO 140 and PA14 EC₅₀ and EC₉₀ values
for HIV-2 and HIV-1.

| | | EC₅₀, μg/ml | | EC₉₀, μg/ml | |
|---|---|---|---|---|---|
| | Tropism | PRO 140 | PA14 | PRO 140 | PA14 |
| HIV-1 | | | | | |
| Isolate | | | | | |
| CC 1/85 | R5 | 0.14 | <0.05 | 6.0 | 2.7 |
| JR-FL | R5 | 0.09 | 0.46 | 7.0 | >100 |
| HIV-2 | | | | | |
| Isolate | | | | | |
| A2267 | R5 | 0.10 | 0.17 | 98 | 8.3 |
| A2270 | R5 | 0.12 | 3.2 | 3.5 | 4.6 |
| 310072 | R5 | 0.08 | 0.07 | 1.4 | 4.4 |
| SLRHC | R5 | 0.57 | 0.35 | 21 | 14 |
| 310340 | R5 | 4.7 | 7.6 | >100 | 63 |
| Median | | 0.12 | 0.35 | 21 | 8.2 |
| 310342 | R5X4 | >100 | >100 | >100 | >100 |
| 77618 | R5X4 | >100 | >100 | >100 | >100 |

Activity of PRO 140 and PA14 against HIV-1 reference isolates and comparison with previous data: Two well-characterized HIV-1 isolates, JR-FL and CC 1/85, were tested for their susceptibility to inhibition by PRO 140. EC₅₀ and EC₉₀ values were determined and compared to results from prior studies (Tables 17 and 18). All EC₅₀ and EC₉₀ values obtained in the current study can be found in Tables 19-21.

TABLE 17
EC₅₀ and EC₉₀ values (ug/ml) observed for PRO 140
against R5 HIV-1 reference viruses.

| | |
|---|---|
| Current Study | Previous Studies |
| (n = 5) | (n = 33) |

| Virus | | EC50 | EC90 | EC50 | EC90 |
|---|---|---|---|---|---|
| JR-FL | Median: | 0.09 | 7.0 | 0.13 | 3.4 |
| | Mean: | <0.97 | 22 | <0.41 | >10 |
| | St Dev: | >1.8 | 36 | >0.62 | >15 |

(n = 24)

| | | | | | |
|---|---|---|---|---|---|
| CC 1/85 | Median: | 1.4 | 6.0 | 0.69 | 4.9 |
| | Mean: | <7.7 | 23 | <1.6 | >12 |
| | St Dev: | >16 | 38 | >2.2 | 9.52 |

TABLE 18
PRO 140 EC₅₀ and EC₉₀ values (ug/ml) for reference HIV-1
isolates from a previous study (300-TD-028), where HIV-1
replication was quantitated by p24 ELISA

| | |
|---|---|
| JR-FL | CC 1/85 |

| Source | EC₅₀ | EC₉₀ | EC₅₀ | EC₉₀ |
|---|---|---|---|---|
| NT040805TK | <0.1 | 1.2 | 0.08 | 0.88 |
| NT041505TK | 0.43 | 34 | 0.1 | 3.4 |
| NT051005TK | 0.37 | 3.4 | 1.1 | 13 |
| NT071204AP-A | 0.003 | 0.01 | 0.004 | 0.34 |
| NT071204AP-B | 0.03 | 18 | 1.4 | 4.2 |
| NT071604TK-A | 0.28 | 5.4 | ND | ND |
| NT071604TK-B | 0.13 | 2.2 | 0.16 | 3.6 |
| NT071604TK-C | 0.05 | 3.1 | ND | ND |
| NT071604TK-D | 0.97 | 12 | ND | ND |
| NT071604TK-P | 0.39 | 3.1 | 2.1 | 9.6 |
| NT071904AP-A | <0.001 | 0.02 | 0.006 | 0.22 |
| NT071904AP-B | <0.001 | <0.001 | <0.001 | <0.001 |
| NT071904AP-C | <0.001 | <0.001 | 0.001 | 0.01 |
| NT071904AP-D | 0.001 | 0.13 | 0.01 | 0.03 |
| NT071904AP-P | <0.001 | 0.02 | <0.001 | 1.1 |
| NT072304TK-A | 0.03 | 0.52 | 4.5 | >50 |
| NT072304TK-B | 1.8 | >50 | ND | ND |
| NT072304TK-C | 0.24 | 3.1 | ND | ND |
| NT072304TK-D | 0.14 | 0.93 | ND | ND |
| NT072304TK-P | 0.58 | 5.8 | 0.7 | 4.9 |
| NT073004TK-D | 0.45 | >50 | 0.96 | >50 |
| NT073004TK-P | 0.06 | 8.1 | 0.67 | 1.1 |
| NT080604TK-A | 0.72 | 8.2 | 0.58 | 8.1 |
| NT080604TK-C | 0.13 | 2.1 | ND | ND |
| NT080604TK-E | 1.9 | 18 | 5.4 | 24 |
| NT080604TK-P | 0.61 | 10 | 4.6 | 33 |
| NT080904AP-A | 0.69 | 11 | ND | ND |
| NT080904AP-B | 0.12 | 2.2 | ND | ND |
| NT080904AP-C | 0.02 | 0.22 | 0.04 | 4.9 |
| NT080904AP-P | 2.7 | 3.7 | 0.85 | 7.3 |
| NT081704AP-A | 0.48 | >50 | 7.8 | 13 |
| NT081704AP-C | 0.06 | 22 | 5.1 | 8.2 |
| NT081704AP-D | 0.04 | 18 | 2.7 | >50 |
| Median: | 0.13 | 3.4 | 0.69 | 4.9 |
| Mean: | <0.41 | >10 | <1.6 | >12 |
| St Dev: | >0.62 | >15 | >2.2 | >17 |

TABLE 19
PRO140 and PA14 EC (ug/ul) for HIV2 and HIV-1

| | RT | PRO 140 | | | | PA14 | | | |
|---|---|---|---|---|---|---|---|---|---|
| Source | (pg/well) | EC₅₀ | EC₉₀ | MPI | R² | EC₅₀ | EC₉₀ | MPI | R² |

Virus: R5 HIV-1 CC 1/85

| | | | | | | | | | |
|---|---|---|---|---|---|---|---|---|---|
| NT111406TK | 0.039 | <0.046 | 5.971 | 102 | 0.753 | <0.046 | 2.726 | 103 | 0.785 |
| NT111706TK | 0.018 | 36.943 | 90.136 | 484 | 0.772 | 27.699 | 87.000 | 210 | 0.794 |
| NT120406TK | 0.444 | 0.135 | 2.578 | 95 | 0.966 | <0.046 | 1.121 | 100 | 0.990 |
| NT121106JZ | 0.251 | 0.054 | 0.199 | 94 | 0.929 | <0.046 | 0.385 | 99 | 0.946 |
| NT010207TK | 0.131 | 1.667 | 15.539 | 93 | 0.842 | 0.668 | 13.416 | 107 | 0.901 |
| Median: | 0.131 | 0.135 | 5.971 | 95 | | 0.046 | 2.726 | 103 | |

Virus: R5 HIV-1 JR-FL

| | | | | | | | | | |
|---|---|---|---|---|---|---|---|---|---|
| NT111406TK | 0.044 | <0.046 | 1.515 | 100 | 0.9392 | <0.046 | 3.815 | 135 | 0.6751 |
| NT111706TK | 0.017 | 34.328 | >100 | 467 | 0.582 | 32.261 | >100 | 102 | 0.7984 |
| NT120406TK | 0.157 | <0.046 | 0.499 | 103 | 0.9976 | <0.046 | 2.300 | 95 | 0.8924 |
| NT121106JZ | 0.102 | 0.134 | 12.426 | 101 | 0.993 | 0.129 | >100 | 88 | 0.9577 |
| NT010207TK | 0.164 | 3.638 | 75.185 | 110 | 0.7915 | 0.792 | >100 | 87 | 0.9715 |
| Median: | 0.102 | 0.090 | 6.971 | 102 | | 0.460 | >100 | 92 | |

Virus: R5 HIV-2 A2267

| | | | | | | | | | |
|---|---|---|---|---|---|---|---|---|---|
| NT111406TK | 0.025 | 0.047 | 0.057 | 97 | 0.9319 | <0.046 | 0.358 | 98.56 | 0.7626 |
| NT111706TK | 0.014 | 4.597 | 98.390 | 115 | 0.7732 | 9.011 | >100 | 83.12 | 0.799 |
| NT120406TK | 0.154 | 0.103 | >100 | 88 | 0.7183 | 0.303 | 15.533 | 121.5 | 0.8465 |
| NT121106JZ | 0.197 | <0.046 | 1.468 | 94 | 0.8508 | <0.046 | 0.988 | 98.82 | 0.7686 |
| NT010207TK | 0.220 | 0.203 | >100 | 75 | 0.992 | 1.431 | >100 | 70.55 | 0.2136 |
| Median: | 0.154 | 0.103 | 98.390 | 94 | | 0.174 | 8.261 | 99 | |

RT: Reverse Transcriptase activity, mean of 6 wells, defined as 100% viral replication
MPI: Maximum percent inhibition as determined from the plateau of the sigmoidal curve fit by Prism software.
Values not included in Table 19 medians (Poor curve fit (R² < 0.7) include the first line of values for PA14/R5 HIV-1 JR-FL, the second line of values PRO140/R5 HIV-1 JR-FL, and the fifth line of values for PA14/R5 HIV-2 A2267.

TABLE 20
PRO140 and PA14 EC (ug/ul) for HIV2 and HIV-1 (continued)

| | RT | PRO 140 | | | | PA14 | | | |
|---|---|---|---|---|---|---|---|---|---|
| Source | (pg/well) | EC₅₀ | EC₉₀ | MPI | R² | EC₅₀ | EC₉₀ | MPI | R² |

Virus: R5 HIV-2 A2270

| | | | | | | | | | |
|---|---|---|---|---|---|---|---|---|---|
| NT111406TK | 0.017 | 0.120 | 0.838 | 103 | 0.867 | <0.046 | 8.021 | 107 | 0.911 |
| NT111706TK | 0.015 | <0.046 | 3.535 | 97 | 0.939 | 3.326 | 9.832 | 99 | 0.955 |
| NT120406TK | 0.040 | 0.363 | 4.886 | 97 | 0.967 | 3.170 | 4.247 | 93 | 0.814 |
| NT121106JZ | 0.157 | <0.046 | 0.788 | 103 | 0.739 | <0.046 | 0.809 | 98 | 0.999 |
| NT010207TK | 0.097 | 0.382 | 9.678 | 101 | 0.971 | 3.531 | 4.614 | 92 | 0.818 |
| Median: | 0.040 | 0.120 | 3.535 | 101 | | 3.170 | 4.614 | 98 | |

Virus: R5 HIV-2 310072

| | | | | | | | | | |
|---|---|---|---|---|---|---|---|---|---|
| NT111406TK | 0.026 | <0.046 | <0.046 | 92 | — | 0.078 | >100 | 90 | 0.997 |
| NT111706TK | 0.032 | 0.576 | 2.057 | 96 | 0.993 | 2.174 | 7.528 | 97 | 0.956 |
| NT120406TK | 0.291 | 0.078 | 0.561 | 96 | 0.890 | 0.063 | 0.411 | 91 | 0.896 |
| NT121106JZ | 0.195 | <0.046 | 1.360 | 97 | 0.969 | <0.046 | 1.327 | 94 | 0.882 |
| NT010207TK | 0.166 | 3.103 | 9.664 | 97 | 0.822 | 3.848 | 4.613 | 95 | 0.676 |
| Median: | 0.166 | 0.078 | 1.360 | 96 | | 0.070 | 4.428 | 92 | |

Virus: R5 HIV-2 SLRHC

| | | | | | | | | | |
|---|---|---|---|---|---|---|---|---|---|
| NT111406TK | 0.049 | <0.046 | 0.337 | 104 | 0.900 | <0.046 | 0.896 | 101 | 0.737 |
| NT111706TK | 0.014 | 0.831 | 32.336 | 135 | 0.977 | 7.288 | 22.063 | 100 | 0.942 |
| NT120406TK | 0.061 | 0.567 | 14.968 | 127 | 0.836 | 0.351 | 13.730 | 103 | 0.860 |
| NT121106JZ | 0.123 | 0.552 | 21.184 | 150 | 0.861 | <0.046 | 10.660 | 100 | 0.832 |
| NT010207TK | 0.111 | 4.647 | 40.089 | 228 | 0.965 | 3.688 | >100 | 97 | 0.873 |
| Median: | 0.061 | 0.567 | 21.184 | 135 | | 0.351 | 13.730 | 100 | |

RT: Reverse Transcriptase activity, mean of 6 wells, defined as 100% viral replication
MPI: Maximum percent inhibition as determined from the plateau of the sigmoidal curve fit by Prism software.
Values not included in Table 20 medians (Poor curve fit (R² < 0.7) include the fifth line of values for PA14/R5 HIV-2 310072.

TABLE 21
PRO140 and PA14 EC (ug/ul) for HIV2 and HIV-1 (continued)

| | RT | PRO 140 | | | | PA14 | | | |
|---|---|---|---|---|---|---|---|---|---|
| Source | (pg/well) | EC₅₀ | EC₉₀ | MPI | R² | EC₅₀ | EC₉₀ | MPI | R² |

Virus: R5 HIV-2 310340

| | | | | | | | | | |
|---|---|---|---|---|---|---|---|---|---|
| NT111406TK | 0.051 | <0.046 | <0.046 | 87 | 0.202 | <0.046 | <0.046 | — | — |
| NT111706TK | 0.024 | 13.862 | >100 | 90 | 0.941 | 15.137 | >100 | 181 | 0.876 |
| NT120406TK | 0.135 | 0.053 | >100 | 78 | 0.822 | 0.114 | 1.641 | 179 | 0.559 |
| NT121106JZ | 0.663 | 0.052 | >100 | 87 | 0.806 | <0.046 | 26.304 | 107 | 0.873 |
| NT010207TK | 0.278 | 9.437 | >100 | 74 | 0.861 | 12.121 | >100 | 83 | 0.515 |
| Median: | 0.135 | 4.745 | >100 | 83 | | 7.591 | 63.152 | 144 | |

Virus: R5X4 HIV-2 310342

| | | | | | | | | | |
|---|---|---|---|---|---|---|---|---|---|
| NT111406TK | 0.230 | >100 | >100 | NA | — | >100 | >100 | NA | — |
| NT111706TK | 0.018 | >100 | >100 | NA | — | >100 | >100 | NA | — |
| NT120406TK | 0.114 | >100 | >100 | NA | — | >100 | >100 | 14 | — |
| NT121106JZ | 0.793 | >100 | >100 | NA | — | >100 | >100 | NA | — |
| NT010207TK | 0.096 | >100 | >100 | NA | — | >100 | >100 | −21 | — |
| Median: | 0.114 | >100 | >100 | — | | >100 | >100 | −4 | |

Virus: R5X4 HIV-2 77618

| | | | | | | | | | |
|---|---|---|---|---|---|---|---|---|---|
| NT111406TK | 0.023 | >100 | >100 | 6 | — | >100 | >100 | 4 | — |
| NT111706TK | 0.017 | >100 | >100 | NA | — | >100 | >100 | NA | — |
| NT120406TK | 0.129 | >100 | >100 | NA | — | >100 | >100 | NA | — |
| NT121106JZ | 0.203 | >100 | >100 | −2 | — | >100 | >100 | −20 | — |
| NT010207TK | 0.072 | >100 | >100 | NA | — | >100 | >100 | −16 | — |
| Median: | 0.072 | >100 | >100 | 2 | | >100 | >100 | −16 | |

Median Values, all R5 HIV-2 Viruses

| | | | | | | | |
|---|---|---|---|---|---|---|---|
| 0.120 | 21.184 | 96 | — | 0.351 | 8.261 | 99 | — |

RT: Reverse Transcriptase activity, mean of 6 wells, defined as 100% viral replication
MPI: Maximum percent inhibition as determined from the plateau of the sigmoidal curve fit by Prism software.
Values not included in Table 21 medians (Poor curve fit (R² < 0.7) include the first line of values for PRO140/R5 HIV-2 310340, and first, third and fifth line of values for PA14/R5 HIV-2 310340.
NA: Not applicable; Prism could not fit a sigmoidal curve.

The EC values were measured by p24 ELISA in previous studies, according to published methods. Because the p24 ELISA cannot efficiently quantitate p24 from the HIV-2 isolates used in this study, a commercial ELISA that measures reverse transcriptase was chosen as a readout for both the HIV-2 and HIV-1 infection assays in the present study. As indicated in Table 17, comparable results were observed using the two assay methods.

### Susceptibility of HIV-2 Isolates to PRO 140 and PA 14:

The susceptibility of five R5 HIV-2 isolates to PRO 140 and PA14 was determined alongside the two HIV-1 reference isolates and two R5X4 HIV-2 isolates as controls. The panel was tested five times on a different PBMC donor pool each time, using the RT ELISA as a readout. The median EC₅₀ values are shown in FIG. 21. Full EC₅₀ and EC₉₀ data can be found in Tables 19-21 above. Against the R5 HIV-2 isolates, PRO 140 had a median EC₅₀ of 0.12 ug/ml, a median EC₉₀ of 21 ug/ml, and a median MPI of 96% (FIGS. 22A-C). These results are similar to those observed for the two R5 HIV-1 reference isolates that were tested in parallel. PRO 140 did not measurably inhibit replication of R5X4 HIV-2 isolates in this study. PRO 140 and PA14 demonstrated similar antiviral activities.

### REFERENCES

U.S. Pat. No. 4,816,567, issued Mar. 28, 1989 to Cabilly et al.
U.S. Pat. No. 5,225,539, issued Jul. 6, 1993 to Gregory Winter.
U.S. Pat. No. 5,229,275, issued Jul. 20, 1993 to Goroff.
U.S. Pat. No. 5,545,806, issued Aug. 13, 1996 to Lonberg et al.
U.S. Pat. No. 5,545,807, issued Aug. 13, 1996 to Surani et al.
U.S. Pat. No. 5,565,332, issued Oct. 15, 1996 to Hoogenboom et al.
U.S. Pat. No. 5,567,610, issued Oct. 22, 1996 to Borrebaeck et al.
U.S. Pat. No. 5,585,089, issued Dec. 17, 1996 to Queen et al.
U.S. Pat. No. 5,591,669, issued Jan. 7, 1997 to Krimpenfort et al.
U.S. Pat. No. 5,693,761, issued Dec. 2, 1997 to Queen et al.
U.S. Pat. No. 6,150,584, issued Nov. 21, 2000 to Kucherlapati et al.
U.S. Pat. No. 6,476,034 B2, issued Nov. 5, 2002 to Wang et al.
U.S. Pat. No. 6,759,519 B2, issued Jul. 6, 2004 to Li et al.
PCT International Publication No. WO 90/07861, published Jul. 26, 1990.
PCT International Publication No. WO 00/35409, published Jun. 22, 2000.
PCT International Publication No. WO 01/55439, published Aug. 2, 2001.
PCT International Publication No. WO 01/90106 A2, published Nov. 29, 2001.
PCT International Publication No. WO 02/22077, published Mar. 21, 2002.
PCT International Publication No. WO 01/62255 A1, published Aug. 30, 2001.
PCT International Publication No. WO 03/082289 A1, published Oct. 9, 2003.
Alkhatib, G., et al. (1996) Science 272:1955.
Allaway, G. P., et al. (1993) AIDS Res. Hum. Retrovir. 9: 581-587.
Allaway, G. P., et al. (1995) AIDS Research and Human Retroviruses. 11: 533-539.
Baba, M., et al. (2005) 12th Conference on Retroviruses and Opportunistic Infections. Boston, Mass., Feb. 22-25, 2005, Abstract 541.
Baba, M., et al. (1999) Proc. Natl. Acad. Sci. USA 96: 5698-5703.
Balotta, C, P. et al. (1997) AIDS 11: F67-F71.
Basavapathruni, A., et al. (2004) J Biol. Chem. 279:6221-6224
Berger, E. A. (1997) AIDS 11 (Suppl A): S3-S16.
Bieniasz, P. D. and B. R. Cullen (1998) Frontiers in Bioscience 3: d44-58.
Biti, R., R. et al. (1997) Nature Med. 3: 252-253.
Borkow, G., et al. (1999) Antimicrob. Agents Chemother. 43:259-263
Burkly, L., et al. (1992) J. Immunol. 149: 1779-1787.
Burkly, L., et al. (1995) J. Virol. 69: 4267-4273.
Choe, H., et al. (1996) Cell 85: 1135-1148.
Chou, T. C. and D. C. Rideout (1991) Synergism and antagonism in chemotherapy. Academic Press, New York.
Chou, T. C. and P. Talalay (1984) Adv. Enzyme Regulation 22: 27-55.
Cocchi, F., et al. (1995) Science 270: 1811-1815.
Combadiere, C, et al. (1996) J. Leukocyte Biol. 60: 147-152.
Connor, R. I., et al. (1997) J. Exp. Med. 185: 621-628.
Cormier, E. G. and T. Dragic (2002) J. Virol. 76:8953-8957.
Cudeck, R. and L. L. O'Dell (1994) Psychol. Bull. 115:475-487.
Dalgleish, A. G., et al. (1984) Nature 312: 763-766.
Demarest, J., et al. (2004) 11th Conference on Retroviruses and Opportunistic Infections, Abstract 139.
San Francisco, Calif., Feb. 8-11, 2004.
Deng, H., et al. (1996) Nature 381: 661-666.
Dorr, P., et al. (2003) 10th Conference on Retroviruses and Opportunistic Infections, Boston, Mass., Feb. 10-14, 2003, Paper #12.
Dorr, P., et al. (2005) Antimicrobial Agents and Chemotherapy 49:4721-4732.
Dragic, T., et al. (1997) Advances in Research and Therapy 7: 2-13.
Dragic, T., et al. (1992) J. Virol. 66: 479-44802.
Dragic, T., et al. (1996) Nature 381: 667-673.
Dragic, T., et al. (2000) Proc Natl Acad Sci USA 97:5639-44.
Este J A. (2002) Curr. Opin. Investig. Drugs. 3: 379-383.
Falkenheuer, G., et al. (2005) Nat Med 11:1170-1172.
Feng, Y., et al. (1996) Science 272: 872-877.
Finke, P. E. et al. (2001) Bioorg. Med. Chem. Lett. 11: 2475-2479.
Garzino-Demo, A., et al. (1999) Proc Natl Acad Sci USA. 96:11986-11991.
Hale, J. J. et al. (2001) Bioorg. Med. Chem. Lett. 11: 2741-2745.
Hale, J. J. et al. (2002) Bioorg. Med. Chem. Lett. 12: 2997-3000.
Hegde, V. R. et al. (2004) Bioorg. Med. Chem. Lett. 12: 5339-5342.
HGS Press Release (2004) Human Genome Sciences characterizes panel of novel human monoclonal antibodies that specifically antagonize the CCR5 receptor and block HIV-1 entry. Nov. 2, 2004.
HGS Press Release (2005) Human Genome Sciences begins dosing of patients in a phase 1 clinical trial of CCR5 mAb in patients infected with HIV-1. Mar. 30, 2005.
Huang, Y., et al. (1996) Nature Med. 2: 1240-1243.
Huffnagle, G. B., et al. (1999) Immunol. 163: 4642-4646.
Y. Iizawa, et al. (2003) 10th Conference on Retroviruses and Opportunistic Infections. Boston, Mass., Feb. 10-14, 2003
Imamura, S. et al. (2004a) Bioorg. Med. Chem. 12: 2295-2306.
Imamura, S. et al. (2004b) Chem. Pharm. Bull. (Tokyo) 52: 63-73.
Imamura, S. et al. (2005) Bioorg. Med. Chem. 13: 397-416.
Jayasuriya, H. et al. (2004) J. Nat. Prod. 67: 1036-1038.
Johnson, V. A., et al. (1991) Journal of Infectious Diseases 164:646-655.
Kawamura, T., et al. (2000) J Exp Med. 192:1491-1500.
Kuhmann, S. E., et al. (2004) J Virol 78:2790-2807.
Ketas, T. J., et al. (2003) J. Virol. 77: 2762-2767.
Kim D. et al. (2001a) Bioorg. Med. Chem. Lett. 11: 3099-3102.
Kim D. et al. (2001b) Bioorg. Med. Chem. Lett. 11: 3103-3106.
Kim D. et al. (2005) Bioorg. Med. Chem. Lett. 15: 2129-2134.
Klatzmann, D., et al. (1984) Nature 312: 382-385.
Koyanagi, Y., et al. (1987) Science 236: 819-822.
Kuhmann, S. E. et al. (2004) J. Virol. 78: 2790-2807.
Kumar, S et al. (2003) J. Pharmacol. Exp. Ther. 304: 1161-1171.
Laal, S., et al. (1994) J. Virol. 68: 4001-4008.
Lalezari, J. P., et al. (2003) New Engl. J. of Med. 348: 2175-2185.
Lalezari, J., et al. (2004) 44th Annual Interscience Conference on Antimicrobial Agents and Chemotherapy, Abstract 2871, Washington, D.C., Oct. 30-Nov. 2, 2004.
Lalezari, J., et al. (2005) AIDS 19:1443-1448.
Lapidot, T. (2001) Ann. N.Y. Acad. Sci. 938: 83-95.
Lazzarin, A., et al. (2003) New Engl. J. Med. 348: 2186.
Lee, B., et al. (1999) Journal of Biological Chemistry 274:9617-9626.
Li, A., et al. (1997) AIDS Res. Hum. Retrovir. 13: 647-656.
Li, A., et al. (1998) J. Virol. 72: 3235-3240.
Lin, P. F., et al. (2003) Proc. Natl. Acad. Sci. USA 100: 11013-11018.
Lin, P. F., et al. (2002) 9th Conference on Retroviruses and Opportunistic Infections. Seattle, Wash., Feb. 24-28, 2002
Littman, D. R. (1998) Cell 93: 677-680.
Litwin, V., et al. (1996) J. Virol. 70: 6437-6441.
Liu, R., et al. (1996) Cell 86: 367-377.
Liu, H., et al. (1999) Proceedings of the National Academy of Sciences of the United States of America 96:4581-4585
Lynch, C. L. et al. (2003a) Bioorg. Med. Chem. Lett. 12: 3001-3004.
Lynch, C. L. et al. (2003b) Bioorg. Med. Chem. Lett. 13: 119-123.
Lynch, C. L. et al. (2002) Bioorg. Med. Chem. Lett. 12: 677-679.
Lynch, C. L. et al. (2003c Org. Lett. 5: 2473-2475.
Maddon, P. J., et al. (1986) Cell 47: 333-348.
Maeda, K. et al. (2004) J. Virol. 78: 8654-8662.
Maeda, K. et al. (2001) J. Biol. Chem. 276: 35194-35200.
Marozsan, A. J. et al. (2005) Virology 338: 182-199.
McCombie, S. W. et al. (2001) Bioorg. Med. Chem. Lett. 13: 567-571.
McDougal, J. S., et al. (1986) Science 231: 382-385.
Merluzzi, V. J., et al. (1990) Science 250: 1411-1413.
Michael, N. L., et al. (1997) Nature Med. 3: 338-340.
Molla, A., et al. (2002) Antimicrob. Agents Chemother. 46:2249-2253.
Moore, J. P., Q. J. Sattentau, P. J. Klasse and L. C. Burkly (1992) J. Virol. 66: 4784-4793.
Nagashima, K. A., et al. (2001) J. Infect. Dis. 183: 1121-1125.
Nakata, H. et al. (2005) J. Virol. 79: 2087-2096.
Nishikawa, M., et al. (2005) Antimicrob. Agents Chemother. 49:4708-4715.
O'Brien, T. R., et al. (1997) Lancet 349: 1219.
Olson, W. C, et al. (1999) J. Virol. 73: 4145-4155.
Olson, W. C. and P. J. Maddon (2003) Current Drug Targets—Infectious Disorders 3:283-294.
Palani, A, et al. (2002) J. Med. Chem. 45: 3143-3160.
Palani, A, et al. (2001) J. Med. Chem. 44: 3339-3342.
Palani, A, et al. (2003a) Bioorg. Med. Chem. Lett. 13: 705-708.
Palani, A, et al. (2003b) Bioorg. Med. Chem. Lett. 13: 709-712.
Palella, F. J., et al. (1998) The New England Journal of Medicine 338:853.
Raport, C. J., et al. (1996) J. Leukocyte Biol. 59: 18-23.
Ray, N. and R. W. Doms (2006) Curr. Top. Microbiol. Immunol. 303:97-120.
Report of an FDA/FCHR Joint Public Meeting, May 31, 2006, Forum for Collaborative HIV Research, Apr. 24, 2007, Department of Prevention and Community Health, The George Washington University School of Public Health and Health Services, Written by J. Fry and J. B. Grossman; Ed. V. Miller.
Reyes, G. (2001) Development of CCR5 antagonists as a new class of anti-HTV therapeutic. 8th Conference on Retroviruses and Opportunistic Infections. Chicago, Ill., Feb. 5, 2001.
Reynes, J., et al. (2002) SCH C: Safety and antiviral effects of a CCR5 receptor antagonist in HIV-1 infected subjects. 9th Conference on Retroviruses and Opportunistic Infections. Seattle, Wash., Feb. 25, 2002
Robinson, B. S., et al. (2000) Antimicrob. Agents Chemother. 44: 2093-2099.
Roschke, V., et al. (2004) 44th Annual Interscience Conference on Antimicrobial Agents and Chemotherapy, Abstract 2871, Washington, D.C., Oct. 30-Nov. 2, 2004, Abstract #2871.
Samson, M., et al. (1997) J. Biol. Chem. 272: 24934-24941.
Schecter, A. D., et al. (2000) J. Biol. Chem. 275: 5466-5471.
Schols, D., et al. (1997) J. Ex. Med. 186: 1383-1388.
Schuh, J. M., et al. (2002) FASEB J. 16: 228-230.
Schurmann, D., et al. (2004) Abstract 140LB, San Francisco, Calif., Feb. 8-11, 2004.
Seibert, C., et al. (2006) Virology 349(1):41-54.
Seto, M. et al. (2005) Bioorg. Med. Chem. Lett. 13: 363-386.
Seto, M. et al. (2004a) Chem. Pharm. Bull. (Tokyo). 52: 818-829.
Seto, M. et al. (2004b) Chem. Pharm. Bull. (Tokyo). 52: 577-590.
Shah, S. K. et al. (2005) Bioorg. Med. Chem. Lett. 15: 977-982.
Shankaran, K. et al. (2004a) Bioorg. Med. Chem. Lett. 14: 3589-3593.
Shankaran, K. et al. (2004b) Bioorg. Med. Chem. Lett. 14: 3419-3424.
Shen, D. M. et al. (2004a) Bioorg. Med. Chem. Lett. 14: 935-939.
Shen, D. M. et al. (2004b) Bioorg. Med. Chem. Lett. 14: 941-945.
Shiraishi, M., et al. (2000) J. Med. Chem. 43: 2049-2063.
Shu, M. et al. (2004) Bioorg. Med. Chem. Lett. 14: 947-52.
Si, Z., et al. (2004) Proc. Natl. Acad. Sci. USA 101: 5036-5041.
Simmons, G., et al. (1996) J. Virol. 70: 8355-8360.
Spenlehauer, C., et al. (2001) Virology 280:292-300.
Strizki, J. M. et al. (2001) Proc. Natl. Acad. Sci. USA. 98: 12718-12723.
Tagat, J. R. et al. (2001a) J. Med. Chem. 44: 3343-3346.
Tagat, J. R. et al. (2001b) Bioorg. Med. Chem. Lett. 11: 2143-2146.
Tagat, J. R., et al. (2004) J. Med. Chem. 47: 2405-2408.
Takashima, K., et al. (2005) Antimicrob. Agents Chemother. 49:374-3482.
Thali, M., et al. (1992) J. Acquir. Immune Defic. Syndr. 5: 591-599.
Thoma, G. et al. (2004) J. Med. Chem. 47: 1939-1955.
Tilley, S. A., et al. (1992) AIDS Res. Hum. Retrovir. 8: 461-467.
Tran, E. H., et al. (2000) Eur. J. Immunol. 30: 1410-1415.
Tremblay, C., et al. (2000) Journal of Acquired Immune Deficiency Syndromes and Human Retrovirology 25:99-102
Tremblay, C. L., et al. (2002) Antimicrobal Agents and Chemotherapy 46:1336-1339.
Tremblay, C. L., et al. (2005) 12th Conference on Retroviruses and Opportunistic Infections. Boston, Mass., Feb. 22-25, 2005, Abstract 542.
Tremblay, C. L., et al. (2005) Antivir. Ther. 10:967-968.
Tremblay, C. L., et al. (2005) Antimicrob. Agents Chemother. 49:3483-3485.
Trkola, A., et al. (2001) J. Virol. 75: 579-588.
Trkola, A., et al. (1999) Journal of Virology 73:8966-8974.
Trkola, A., et al. (1998) J. Virol. 72: 1876-1885.
Tsamis, F., et al. (2003) Journal of Virology 77:5201-5208.
Vijh-Warrier, S., et al. (1996) J. Virol. 70: 4466-4473.
Watson, C., et al. (2005) Mol. Pharmacol. 67:1268-1282.
Whitcomb, J. M., et al. (2007) Antimicrob. Agents Chemother. 51:566-575
Wild, C., et al. (1992) PNAS 89:10537-10541.
Willoughby, C. A. et al. (2001) Bioorg. Med. Chem. Lett. 11: 3137-41.
Willoughby, C. A. et al. (2003) Bioorg. Med. Chem. Lett. 13: 427-431.
Wu, L., et al. (1997) J. Exp. Med. 186: 1373-1381.
Zhou, Y., et al. (1998) J. Immunol. 160: 4018-4025.
Zhu, P., et al. (2001) J. Virol. 75: 6682-6686.

1. A method of inhibiting HIV-2 infection of a susceptible cell by HIV-2 in a subject which comprises subjecting the susceptible cell to an effective HIV-2 infection inhibiting dose of (a) a humanized antibody designated PRO 140, or of (b) an anti-CCR5 receptor monoclonal antibody which (i) binds to CD4+CCR5+ cells and inhibits fusion of HIV-1 with such cells, (ii) inhibits HIV-1 fusion with CD4+CCR5+ cells with a potency equal or greater than that of PRO 140, (iii) coats CD4+CCR5+ cells in the subject without reducing the number of such cells in the subject, and/or (iv) binds to the subject's CD4+CCR5+ cells without inducing an increase in the subject's plasma concentration of circulating β-chemokines, wherein PRO 140 comprises (i) two light chains, each light chain comprising the light chain variable (V_{L}) and constant (C_{L}) regions encoded by the plasmid designated pVK:HuPRO140-VK (ATCC Deposit Designation PTA-4097), and (ii) two heavy chains, each heavy chain comprising the heavy chain variable (V_{H}) and constant (C_{H}) regions encoded either by the plasmid designated pVg4:HuPRO140 HG2-VH (ATCC Deposit Designation PTA-4098) or by the plasmid designated pVg4:HuPRO140 (mut B+D+I)-VH (ATCC Deposit Designation PTA-4099), wherein the effective HIV-2 infection inhibiting dose comprises from 0.1 mg per kg to 25 mg per kg of the subject's body weight, so as to thereby inhibit the infection of the susceptible cell by HIV-2.

2. The method of claim 1, wherein the susceptible cell is present in a human subject.

3. The method of claim 1, wherein the anti-CCR5 receptor monoclonal antibody binds to the same CCR5 epitope as that to which PRO 140 binds.

4. The method of claim 1, wherein the anti-CCR5 receptor monoclonal antibody is a humanized, a human, or a chimeric antibody.

5. The method of claim 1, wherein the susceptible cell is subject to an effective HIV-2 infection inhibiting dose of the antibody designated PRO 140.

6. The method of claim 5, wherein the antibody designated PRO 140 comprises (i) two light chains, each light chain comprising the light chain variable (V_{L}) and constant (C_{L}) regions encoded by the plasmid designated pVK:HuPRO140-VK (ATCC Deposit Designation PTA-4097) and (ii) two heavy chains, each heavy chain comprising the heavy chain variable (V_{H}) and constant (C_{H}) regions encoded by the plasmid designated pVg4:HuPRO140 HG2-VH (ATCC Deposit Designation PTA-4098).

7. The method of claim 1, wherein HIV-2 is of a subtype selected from subtypes A, B, C, D, E, F, G, H, J, O, or a combination thereof.

8. (canceled)

9. (canceled)

10. (canceled)

11. (canceled)

12. The method of claim 2, wherein the human subject is administered the antibody designated PRO 140 which comprises (i) two light chains, each light chain comprising the light chain variable (V_{L}) and constant (C_{L}) regions encoded by the plasmid designated pVK:HuPRO140-VK (ATCC Deposit Designation PTA-4097) and (ii) two heavy chains, each heavy chain comprising the heavy chain variable (V_{H}) and constant (C_{H}) regions encoded by the plasmid designated pVg4:HuPRO140 HG2-VH (ATCC Deposit Designation PTA-4098).

13. The method of claim 2, wherein the effective HIV-2 infection inhibiting dose is (1) from 0.25 mg per kg to 25 mg per kg of the subject's body weight; or (2) from 0.5 mg per kg to 25 mg per kg of the subject's body weight; or (3) from 1 mg per kg to 5 mg per kg of the subject's body weight.

14. (canceled)

15. (canceled)

16. The method of claim 2, wherein the effective HIV-2 infection inhibiting dose is (1) 5 mg per kg of the subject's body weight; or (2) 10 mg/kg of the subject's body weight; or (3) 20 mg/kg of the subject's body weight.

17. (canceled)

18. (canceled)

19. The method of claim 2, wherein the effective HIV-2 infection inhibiting dose is administered at regular intervals.

20. The method of claim 2, wherein the effective HIV-2 infection inhibiting dose is sufficient to achieve in the subject a serum concentration of the antibody of at least 400 ng/ml.

21. The method of claim 2, wherein the effective HIV-2 infection inhibiting dose is sufficient to achieve and maintain in the subject a serum concentration of the antibody of (1) at least 1 μg/ml; or (2) about 3 to about 12 μg/ml; or (3) at least 5 μg/ml; or (4) at least 10 μg/ml; or (5) at least 25 μg/ml; or (6) at least 50 μg/ml.

22. (canceled)

23. (canceled)

24. (canceled)

25. (canceled)

26. (canceled)

27. The method of claim 2, wherein the effective HIV-2 infection inhibiting dose is administered at one or more predefined intervals.

28. (canceled)

29. (canceled)

30. (canceled)

31. (canceled)

32. (canceled)

33. (canceled)

34. (canceled)

35. (canceled)

36. (canceled)

37. The method of claim 2, wherein the antibody is administered to the human subject via intravenous infusion or via subcutaneous injection.

38. (canceled)

39. The method of claim 2, further comprising administering to the human subject at least one additional antiretroviral agent effective against HIV-2.

40. The method of claim 39, wherein the antiretroviral agent is a CCR5 antagonist that does not compete with the humanized antibody designated PRO 140 of (a), or the anti-CCR5 receptor monoclonal antibody of (b).

41. The method of claim 40, wherein the CCR5 antagonist is an antibody, a non-antibody, small-molecule CCR5 antagonist.

42. (canceled)

43. The method of claim 41, wherein the CCR5 antagonist is an antibody which is a monoclonal antibody, humanized, a human, or a chimeric antibody.

44. (canceled)

45. (canceled)

46. (canceled)

47. (canceled)

48. A method of (a) inhibiting in a human subject the onset or progression of an HIV-2-associated disorder, or (b) of reducing the likelihood of a human subject's contracting infection by HIV-2, the inhibition of which or reduction being effected by inhibiting fusion of HIV-2 to a susceptible cell in a subject, which comprises administering to the subject at a predefined interval effective fusion-inhibitory doses of a humanized antibody designated PRO 140, or of an anti-CCR5 receptor antibody which (i) binds to CD4+CCR5+ cells and inhibits fusion of HIV-1 with such cells, (ii) inhibits HIV-1 fusion with CD4+CCR5+ cells with a potency equal or greater than that of PRO-140 (iii) coats CD4+CCR5+ cells in the subject without reducing the number of such cells in the subject, and/or (iv) binds to the subject's CD4+CCR5+ cells without inducing an increase in the subject's plasma concentration of circulating β-chemokines, wherein PRO 140 comprises (i) two light chains, each light chain comprising the light chain variable (V_{L}) and constant (C_{L}) regions encoded by the plasmid designated pVK:HuPRO140-VK (ATCC Deposit Designation PTA-4097), and (ii) two heavy chains, each heavy chain comprising the heavy chain variable (V_{H}) and constant (C_{H}) regions encoded either by the plasmid designated pVg4:HuPRO140 HG2-VH (ATCC Deposit Designation PTA-4098) or by the plasmid designated pVg4:HuPRO140 (mut B+D+I)-VH (ATCC Deposit Designation PTA-4099), wherein each administration of the antibody delivers to the subject from 0.1 mg per kg to 25 mg per kg of the subject's body weight, so as to thereby inhibit the onset or progression of the HIV-2-associated disorder in the subject or reduce the likelihood of a human subject's contracting infection by HIV-2.

49. (canceled)

50. (canceled)

51. (canceled)
